SEQUENCE LISTING (40 sequences)

SEQ ID NO: 1 - protein, 11 aa, Artificial
  Exemplary protein transduction domain
YGRKKRRQRRR

SEQ ID NO: 2 - protein, 9 aa, Artificial
  Exemplary protein transduction domain
RKKRRQRRR

SEQ ID NO: 3 - protein, 11 aa, Artificial
  Exemplary protein transduction domain
YARKARRQARR

SEQ ID NO: 4 - protein, 11 aa, Artificial
  Exemplary protein transduction domain
YARAAARQARA

SEQ ID NO: 5 - protein, 11 aa, Artificial
  Exemplary protein transduction domain
YARAARRAARR

SEQ ID NO: 6 - protein, 11 aa, Artificial
  Exemplary protein transduction domain
RARAARRAARA

SEQ ID NO: 7 - protein, 16 aa, Artificial
  Exemplary protein transduction domain
RQIKIWFQNRRMKWKK

SEQ ID NO: 8 - protein, 34 aa, Artificial
  Exemplary protein transduction domain
DAATATRGRSAASRPTERPRAPARSASRPRRPVE

SEQ ID NO: 9 - protein, 7 aa, Artificial
  Exemplary protein transduction domain
RRRRRRR

SEQ ID NO: 10 - protein, 33 aa, Artificial
  Exemplary protein transduction domain
YARAAARQARAYARAAARQARAYARAAARQARA

SEQ ID NO: 11 - DNA, 23 bp, Artificial
  Human Oct4 sense
ttccatggcgggacacctggctt

SEQ ID NO: 12 - DNA, 31 bp, Artificial
  Human Oct4 antisense
ttgaattctcagtttgaatgcatgggagagc

SEQ ID NO: 13 - DNA, 25 bp, Artificial
  Mouse Oct4 sense
ttccatggctggacacctggcttca

SEQ ID NO: 14 - DNA, 31 bp, Artificial; identical to SEQ ID NO: 12
  Mouse Oct4 antisense

SEQ ID NO: 15 - DNA, 28 bp, Artificial
  Human Nanog sense
atactggtaccagtgtggatccagcttg

SEQ ID NO: 16 - DNA, 25 bp, Artificial
  Human Nanog antisense
ttcactcgaattcacacgtcttcag

SEQ ID NO: 17 - DNA, 26 bp, Artificial
  Mouse Nanog sense
gaacgcctcatccatggctgcagttt

SEQ ID NO: 18 - DNA, 24 bp, Artificial
  Mouse Nanog antisense
cagatgttgcggaattctcatatt

SEQ ID NO: 19 - DNA, 26 bp, Artificial
  c-Myc sense
ctcccgcgaccatggccctcaacgtt

SEQ ID NO: 20 - DNA, 27 bp, Artificial
  c-Myc antisense
gacatttctgttagaaggaattctttt

SEQ ID NO: 21 - DNA, 26 bp, Artificial
  Human Sox-2 sense
cgcccgcatgggtaccatgatggaga

SEQ ID NO: 22 - DNA, 26 bp, Artificial
  Human Sox-2 antisense
ctccagttcgaattccggccctcaca

SEQ ID NO: 23 - DNA, 32 bp, Artificial
  Mouse Sox-2 sense
tttttggtaccatgtataacatgatggagacg

SEQ ID NO: 24 - DNA, 31 bp, Artificial
  Mouse Sox-2 antisense
tttttgaattctcacatgtgcgagaggggca

SEQ ID NO: 25 - DNA, 22 bp, Artificial
  Human Klf4 sense
gcgagtctgccatggctgtcag

SEQ ID NO: 26 - DNA, 25 bp, Artificial
  Human Klf4 antisense
cactgtctggaattcaaaaatgcct

SEQ ID NO: 27 - DNA, 29 bp, Artificial
  Mouse Klf4 sense
tttttccatggctgtcagcgacgctctgc

SEQ ID NO: 28 - DNA, 27 bp, Artificial
  Mouse Klf4 antisense
tttttgaattcttaatgcctcttcatg

SEQ ID NO: 29 - protein, 7 aa, Artificial
  SV40 large T Antigen NLS
PKKKRKV

SEQ ID NO: 30 - protein, 6 aa, Artificial
  human retinoic acid receptor-beta nuclear localization signal
ARRRRP

SEQ ID NO: 31 - protein, 10 aa, Artificial
  NF kappa B p50 NLS
EEVQRKRQKL

SEQ ID NO: 32 - protein, 9 aa, Artificial
  NF kappa B p65 NLS
EEKRKRTYE

SEQ ID NO: 33 - protein, 20 aa, Artificial
  Xenopus nucleoplasmin NLS
AVKRPAATKKAGQAKKKKLD

SEQ ID NO: 34 - DNA, 609 bp, Artificial
  pSecTag2 B multiple cloning site
aatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtgacgcggcccagccggccaggcgcgcgcgccgtacgaagcttggtaccgagctcggatccactccagtgtggtggaattctgcagatatccagcacagtggcggccgctcgaggagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgt

SEQ ID NO: 35 - protein, 21 aa, Artificial
  pSecTag2 B Ig kappa-chain leader sequence
METDTLLLWVLLLWVPGSTGD

SEQ ID NO: 36 - protein, 21 aa, Artificial
  pSecTag2 B myc and polyhistidine tags
EQKLISEEDLNSAVDHHHHHH

SEQ ID NO: 37 - DNA, 294 bp, Artificial
  pTAT multiple cloning site
atctcgatcccgcgaaattaatacgactcactatagggagaccacaacggtttccctctagataattttgtttaactttaagaaggagatatacatatgcggggttctcatcatcatcatcatcatggtatggctagcatgactggtggacagcaaatgggtcgggatctgtacgacgatgacgataaggatcgatggggaggctacggccgcaagaaacgccgccagcgccgccgcggtggatccaccatggccggtaccggtctcgaggtgcatgcggtgaattcgaagctt

SEQ ID NO: 38 - protein, 66 aa, Artificial
  pTAT peptide
MRGSHHHHHHGMASMTGGQQMGRDLYDDDDKDRWGGYGRKKRRQRRRGGSTMAGTGLEVHAVNSKL

SEQ ID NO: 39 - DNA, 50 bp, Artificial
  pTAT-HA insertion
ccatgtccggctatccatatgacgtcccagactatgctggctccatgggc

SEQ ID NO: 40 - protein, 16 aa, Artificial
  pTAT-HA insertion peptide
MSGYPYDVPDYAGSMG

(19) United States
(12) Patent Application Publication
(10) Pub. No.: US 2011/0171185 A1
(43) Pub. Date: Jul. 14, 2011

(54) Genetically intact induced pluripotent cells or transdifferentiated cells and methods for the production thereof

(76) Inventors: IRINA V. KLIMANSKAYA, UPTON, MA (US); SHI-JIANG LU, SHREWSBURY, MA (US); ROBERT LANZA, CLINTON, MA (US); MICHAEL D. WEST, MILL VALLEY, CA (US); KAREN B. CHAPMAN, MILL VALLEY, CA (US); ROY GEOFFREY SARGENT, SAN LORENZO, CA (US); RAYMOND PAGE, SOUTHBRIDGE, MA (US); TANJA DOMINKO, SOUTHBRIDGE, MA (US); CHRISTOPHER MALCUIT, WARE, MA (US)

(21) Appl. No.: 12/700,545

(22) Filed: Feb. 4, 2010

Related U.S. Application Data
(63) Continuation-in-part of application No. 12/609,439, filed on Oct. 30, 2009.
Application No. 12/609,439 is a continuation of application No. 10/228,296, filed on Aug. 27, 2002, now Pat. No. 7,621,606.
Application No. 10/228,296 is a continuation-in-part of application No. 11/989,988, filed on Mar. 8, 2010.
Application No. 11/989,988 is a continuation-in-part of application No. 10/831,599, filed on Apr. 23, 2004.
Application No. 10/831,599 is a continuation of application No. 09/736,268, filed on Dec. 15, 2000, now abandoned.
Application No. 09/736,268 is a continuation-in-part of application No. PCT/US00/18063, filed on Jun. 30, 2000.
(60) Provisional application No. 61/181,547, filed on May 27, 2009.
(60) Provisional application No. 60/314,654, filed on Aug. 27, 2001.
(60) Provisional application No. 60/818,813, filed on Jul. 5, 2006.
(60) Provisional application No. 60/729,173, filed on Oct. 20, 2005.
(60) Provisional application No. 60/705,625, filed on Aug. 3, 2005.
(60) Provisional application No. 60/141,250, filed on Jun. 30, 1999.

(51) Int. Cl. A61K 35/12; C12N 15/01; A61P 43/00

(52) U.S. Cl. 424/93.21; 435/440; 424/93.7

(57) ABSTRACT

The present disclosure relates to methods for dedifferentiating and transdifferentiating recipient cells, preferably human somatic cells. These methods minimize the risk of undesired genome sequence alteration. These methods employ reprogramming factors, which may be used alone or in certain combinations with one another. These methods have application especially in the context of cell-based therapies, establishment of cell lines, and the production of genetically modified cells.

### CROSS-REFERENCE TO RELATED APPLICATIONS

This application claims the benefit of U.S. Provisional Application Ser. No. 61/181,547 (Atty. Docket No. 75820.000002), filed May 27, 2009. This application is also a continuation-in-part of U.S. Ser. No. 12/609,439 (Atty. Docket No. 75820.036011), filed Oct. 30, 2009, which is a continuation of U.S. Ser. No. 10/228,296 (Atty. Docket No. 75820.036010), filed Aug. 27, 2002, now U.S. Pat. No. 7,621,606, which claims the benefit of U.S. Provisional Application Ser. No. 60/314,654, filed Aug. 27, 2001. This application is also a continuation-in-part of U.S. Ser. No. 11/989,988 (Atty. Docket No. 75820.005010), filed Feb. 4, 2008, now pending, which is a national stage entry of international application no. PCT/US2006/030643, filed Aug. 3, 2006, which claims the benefit of U.S. Provisional Application Ser. No. 60/818,813, filed Jul. 5, 2006, now expired, U.S. Provisional Application Ser. No. 60/729,173, filed Oct. 20, 2005, now expired, and U.S. Provisional Application Ser. No. 60/705,625, filed Aug. 3, 2005, now expired. This application is also a continuation-in-part of U.S. Ser. No. 10/831,599 (Atty. Docket No. 75820.014011), filed Apr. 23, 2004, now pending, which is a Continuation of U.S. Ser. No. 09/736,268, filed Dec. 15, 2000, now abandoned, which is a continuation-in-part of international application no. PCT/US00/18063, filed Jun. 30, 2000, which claims the benefit of U.S. Provisional Application Ser. No. 60/141,250 filed Jun. 30, 1999. The contents of each of the foregoing is hereby incorporated by reference in its entirety to the extent that they are not inconsistent with the disclosures contained herein.

### BACKGROUND

1. Field of the Art

The present disclosure relates to methods and materials for reprogramming (dedifferentiating and transdifferentiating) recipient cells or recipient cell nuclei, preferably human somatic cells or human somatic cell nuclei. These methods have application especially in the context of cell-based therapies, tissue transplantation, establishment of cell lines, and the production of genetically modified cells and chimeric or transgenic animals.

In another aspect, the present disclosure relates to methods for “de-differentiating” and/or altering the life-span of desired recipient cells, preferably human somatic cells. These methods have application especially in the context of cell therapies and the production of genetically modified cells.

In another aspect, the present disclosure relates to methods for effecting trans-differentiation of somatic cells. Trans-differentiation is the conversion of a cell from one differentiated cell type to another differentiated cell type.

In another aspect, the present disclosure generally relates to methods of reprogramming an animal somatic cell from a particular differentiated state to another state, and the use of such cells and tissues in the treatment of human diseases and age-related conditions. More particularly, the disclosure relates to an improved method utilizing a three-step process whereby the nuclear envelope of the somatic cell nucleus is first remodeled to that of an undifferentiated cell or a germ-line cell prior to the second step of transferring the remodeled nucleus into the cytoplasm of an oocyte or an undifferentiated cell. This nuclear remodeling step markedly enhances the efficiency of cellular reconstitution when the remodeled nucleus is transferred into embryonic or germ-line cytoplasm for the purpose of stem cell derivation. In addition, the removal of components of the nuclear envelope specific for differentiated cells, such as lamin A, and the reprogramming of chromatin results in a reactivation of telomerase activity, a lengthening of telomere length, and mechanisms of homologous recombination that repair tandemly-repeated DNA sequences. When pluripotent stem cells are derived by these methods, they may be utilized in novel therapeutic strategies in the treatment of cardiac, neurological, endocrinological, vascular, retinal, dermatological, muscular-skeletal disorders, and other diseases.

2. Description of Related Art

Advances in stem cell technology, such as the isolation and use of human embryonic stem (hES) cells, have become an important new subject of medical research. hES cells have a demonstrated potential to differentiate into any and all of the cell types in the human body, including complex tissues. This ability of hES cells has led to the suggestion that many diseases resulting from the dysfunction of cells may be amenable to treatment by the administration of hES-derived cells of various differentiated types (Thomson et al., Science. 1998 Nov. 6; 282(5391):1145-7). Nuclear transfer studies have demonstrated that it is possible to transform a somatic differentiated cell back to a totipotent state such as that of ES or ED cells (Cibelli et al., Nat Biotechnol. 1998 July; 16(7):642-6). The development of technologies to reprogram somatic cells back to a totipotent ES cell state such as by nuclear transfer offers a means to deliver ES-derived somatic cells with a nuclear genotype of the patient (Lanza et al., Nat Med. 1999 September; 5(9):975-7). It is expected that such cells and tissues would not be rejected, despite the presence of allogeneic mitochondria (Lanza et al., Nat Biotechnol. 2002 July; 20(7):689-96). Nuclear transfer also allows the rebuilding of telomere repeat length in cells through the reactivation of the telomerase catalytic component in the early embryo (Lanza et al., Science. 2000 Apr. 28; 288(5466):665-9).

Because of the relative difficulty of obtaining sufficient numbers of human oocytes, there has been considerable interest in determining whether other germ-line cells, such as cultured ES cells, or cytoplasm from said cells, could be used to reprogram somatic cells. Such cells would have an important advantage over oocytes as a means of inducing reprogramming in that they can be easily expanded in number in vitro. The restoration of expression of at least some measured embryonic-specific genes has been observed in somatic cells following fusion with ES cells (Do and Scholer, Stem Cells. 2004; 22(6):941-9; Do and Scholer, Reprod Fertil Dev. 2005; 17(1-2):143-9). However, the resulting cells are hybrids, often with a tetraploid genotype, and therefore not suited as normal or histocompatible cells for transplant purposes. Indeed, one of the proposed purposes of generating autologous totipotent cells is to prevent the rejection of ES-derived cells. Using the techniques described in these published studies, the ES cells used to reprogram a patient's cell would therefore likely add alleles that could generate an immune response leading to rejection. Nevertheless, the evidence that ES cells can reprogram somatic cell chromosomes has excited researchers and generated a new field of research called “fusion biology” (Dennis, Nature 426:490-491, (2003)).

However, ES cell research has been impeded by the controversy surrounding the use of unwanted IVF embryos for generation of ES cells and donation of oocytes, which are not intended for fertilization and pregnancy rather but for alternative approaches to produce patient immune-compatible cells for regenerative medicine applications. Many countries now place restrictions on embryonic stem cell research including limitation on the available state funds along with strict guidelines on oocyte and embryo use, resulting in slowed advancements in this field. Moreover, clinical usefulness of ES cell-based therapies will be limited unless a diverse set of histocompatible cells is available to match individual patients.

Another potential source of cells capable of reprogramming human somatic cells with a greater ease of availability than human oocytes are oocytes of animal species. The demonstration of the restoration of totipotency in somatic cells by nuclear transfer across species (Lanza et al., Cloning. 2000; 2(2):79-90) opens the possibility of identifying animal oocytes that can be easily obtained for use in reprogramming human cells (Byrne et al., Curr Biol. 2003 Jul. 15; 13(14):1206-13). However, likely because of molecular differences between the species, cross species nuclear transfer, although possible, is often even less efficient than same-species nuclear transfer.

Therefore, each of these methods for reprogramming human somatic cells have their own difficulties. SCNT provides a satisfactory level of reprogramming but is limited by the number of human oocytes available to researchers. Cross-species nuclear transfer and cell fusion technologies are not generally limited in the cells used in reprogramming but are limited by the degree of successful reprogramming or the robustness of the growth of the resulting reprogrammed cells.

An alternative to the use of donor cell cytoplasm for dedifferentiation is introduction of defined factors. To identify a workable group of dedifferentiation factors, Takahashi and Yamanaka (2006 Aug. 25; 126(4):663-76) introduced candidate genes into mouse embryonic fibroblasts (MEFs) by retroviral transduction. Each retrovirus carried a single candidate gene, and combinations of candidate genes were introduced by multiple infection. The MEFs were also engineered to express a selectable marker under control of the ES cell-specific Fbx15 promoter, such that cell survival in the presence of the antibiotic G418 was dependent on successful dedifferentiation. By testing different combinations of factors, the authors demonstrated a combination of four transcription factor genes (Oct4, Sox2, c-Myc, and Klf4) resulted in ES-like pluripotent cells, which have been called induced pluripotent (iPS) cells. Using a similar approach, Yu et al. (Science. 2007 Dec. 21; 318(5858):1917-20) generated human iPS cells by viral integration of genes encoding Oct4, Sox2, Nanog, And Lin28.

Though retroviral transfection has been an effective means to simultaneously deliver multiple genes into a somatic cell, safety concerns arise from their use for dedifferentiation. Because these methods cause multiple genes to be integrated at multiple sites, targeted techniques for excision of the transgenes (e.g., Cre-Lox and FLP-FRT) are difficult to use, as unintended deletions and other intra-chromosomal and inter-chromosomal genomic rearrangements could result. Moreover, the insertion of retroviral vectors is a potential threat to the integrity of the transfected cell genome, e.g., by affecting non-targeted genes, through integration of undesired viral sequences, and through the aberrant expression of the integrated genes which could contribute to malignancy. Indeed, reactivation of c-Myc carried by a retrovirus resulted in tumor formation in approximately 50% of chimeric mice generated from iPS cells (Okita et al., Nature. 2007 Jul. 19; 448(7151):313-7).

In view of the foregoing, there is need for safe and effective methods to dedifferentiate or transdifferentiate cells. The desired methods avoid the use of embryos or embryo-derived materials, and also avoid undesired genome sequence alteration. Particularly desired are methods that increase the frequency and quality of reprogramming of differentiated somatic cells and of producing reprogrammed cells that are capable of expansion in vitro in order to obtain a useful number of cells for research, testing for quality control, and for use in cell therapy. Preferably these methods provide a practical source of patient-derived cells for therapeutic use.

### SUMMARY

Applicants describe methods and materials for reprogramming or transdifferentiating somatic cells, preferably human somatic cells, which somatic cells optionally may be genetically modified such as too comprise a heterologous nucleic acid sequence, and for producing iPS cells by novel methods that minimize the risk of genome sequence alteration and which increase cell lifespan and reduce senescence. These methods employ compositions comprising or encoding one or more endogenous or recombinant reprogramming factors or functional fragments, variants or fusion polypeptides or cell extracts which contain said endogenous reprogramming factors to “reprogram” a desired donor or recipient cell or cell nucleus or chromosomal DNA thereof, preferably a human somatic cell or nucleus or chromosomal DNA thereof. As defined infra, “reprogramming” in the present disclosure is intended to encompass any method that uses a composition containing or encoding one or more endogenous or recombinant reprogramming factors or functional fragments, variants or fusion polypeptides containing to convert a donor or recipient cell or cell nucleus into a less differentiated or dedifferentiated or rejuvenated cell (e.g., induced pluripotent cell or embryonic stem cell or adult stem cell or cell having an increased lifespan relative to parent cells as evidenced e.g., by increased telomere or increased cell divisions relative to parent cell) or to transdifferentiate the somatic cell or nucleus into a cell or cytoplast containing said nucleus into a cell of a different cell type or lineage. In exemplary embodiments, the reprogramming factors include endogenous or recombinant reprogramming polypeptides or functional fragments, variants or fusion polypeptides containing, which e.g., may be comprised in a donor cell cytoplasm, may be synthesized or produced recombinantly, may optionally include one or more modifications, and may optionally be purified. In certain embodiments the reprogramming polypeptides include one or more of the polypeptides Nanog, Oct4, Sox2, c-Myc, Klf4, and Lin28 or functional fragments, variants or fusions containing. In addition one or more of the reprogramming polypeptides may be coupled to a nucleus or protein translocation domain that facilitates cell entry and/or nuclear translocation.

The rejuvenated or transdifferentiated or reprogrammed cells and cell nuclei created by these methods can be used for many purposes, including for example cell-based therapies and for the expression of heterologous proteins. The cells used for cell-based therapies may be derived from the patient or from a histocompatible donor. Additionally, the cells used for cell-based therapies may be genetically altered to change their histocompatibility profile. As mentioned, the cells optionally include desired genetic modifications, e.g., gene modifications which eliminate gene abnormalities associated with specific genetic diseases such as cystic fibrosis, ALD, sickle cell anemia, cancer, autoimmune disorders and/or genetically modified in order to provide for the expression (constitutive, regulated or tissue specific) of therapeutic polypeptides or immune modulators.

The present disclosure in a preferred embodiment provides novel methods for producing reprogrammed nuclei and cells, preferably mammalian cells and, most preferably, human cells that have been de-differentiated and/or which have an altered (increased) life-span by the juxtaposition or incubating of the donor cell or nucleus thereof with a cell derived extract comprising cytoplasm from an undifferentiated or substantially undifferentiated cell, preferably an oocyte or blastomere, or another embryonic cell type such as an embryonic stem cell. In a particularly preferred embodiment, the present invention will be used to produce cells in a more primitive state, especially embryonic stem cells or inner cell mass cells.

In another aspect, this disclosure provides methods for de-differentiating or altering the life-span of desired “recipient” cells, e.g., human somatic cells, by the introduction of or contacting these cells or nuclei thereof with a composition containing cytoplasm from a more primitive, less differentiated cell type, e.g., oocyte or blastomere or ES cell are provided. In exemplary embodiments these methods can be used to produce embryonic stem cells and to increase the efficiency of gene therapy by allowing for desired cells to be subjected to multiple genetic modifications without becoming senescent. Such cytoplasm may be fractionated and/or subjected to subtractive hybridization and the active materials (sufficient for de-differentiation) identified and produced by recombinant methods.

In another aspect, the present application provides methods for reprogramming, i.e., “de-differentiating” and/or altering the life-span of desired cells, for example by introducing into or contacting a cell or cell nucleus with cytoplasm from another cell, e.g., a less differentiated cell for a time sufficient to effect dedifferentiation or to increase lifespan of the cell or a cell containing this nucleus and then transplanting the de-differentiated cell or nucleus into a surrogate cytoplast such as from an ES cell of a less differentiated cell, preferably an oocyte or blastomere, or another embryonic cell type.

In another aspect, the present application provides methods to alter the life-span and/or to de-differentiate desired cells, typically mammalian differentiated cells, prior, concurrent, or subsequent to genetic modification.

In another aspect, the present application provides an improved method of cell therapy wherein the improvement comprises administering cells which have been de-differentiated or have an altered life-span by the introduction of cytoplasm obtained from a cell of a less or undifferentiated state, preferably an oocyte or blastomere or placing nuclei from said somatic cell into a solution containing an extract of the oocyte or blastomere embryo, or ES cell or purified proteins from the same.

In another aspect, the present application provides for the identification of the component or components in oocyte cytoplasm responsible for de-differentiation and/or alteration of cell life-span, e.g., by fractionation or subtractive hybridization, i.e. fractionation of protein, RNA or DNA.

In another aspect, the present application provides methods of therapy, especially of the skin, by administering a therapeutically effective amount of cytoplasm obtained from a substantially undifferentiated or undifferentiated cell, preferably an oocyte or blastomere, or the purified active components of the same.

In another aspect, the present application provides novel compositions for therapeutic, dermatologic and/or cosmetic usage that contain cytoplasm derived from substantially undifferentiated or undifferentiated cells, preferably an oocyte or blastomere, or purified active components of same.

In another aspect, the present application provides cells for use in cell therapy which have been “de-differentiated” or have an altered life-span by the introduction of a composition comprising cytoplasm from a substantially undifferentiated or undifferentiated cell, preferably an oocyte or blastomere, or purified active components of same.

In another aspect, the present application provides an improved method of cloning via nuclear transfer wherein the improvement comprises using as the donor cell or nucleus a cell which has been de-differentiated and/or has had its life-span altered by contacting or incubating therewith or by the introduction therein of a composition comprising cytoplasm from a substantially undifferentiated or undifferentiated cell, or purified active components of same, or cross-species NT where the purified active component is expressed to facilitate reprogramming.

In another aspect, the present application provides methods of rejuvenating nuclei isolated from desired differentiated cells by contacting same with a composition comprising cytoplasm from oocytes, blastomeres, ES, or other embryonic cell types.

In another aspect, the present application provides screening assays to identify proteins, or nucleic acid sequences that are released from differentiated cell nuclei upon contacting with cytoplasm, or fractions derived from oocyte cytoplasm from oocytes, blastomeres, ES cells or other embryonic cell types, that are involved in all reprogramming.

In another aspect, the present application provides screening assays, e.g. differential or subtractive hybridization to identify mRNAs that expressed in oocyte cytoplasm or in embryonic cell types that are involved in cell programming.

The resultant cells are useful in gene and cell therapies, and as donor cells or nuclei for use in nuclear transfer.

The disclosure also provides a method for effecting the trans-differentiation of a somatic cell or nucleus, i.e., the conversion of a somatic cell or nucleus of one cell type into a somatic cell or nucleus of a different cell type. The method can be practiced by culturing a somatic cell or nucleus in the presence of at least one agent selected from the group consisting of (a) cytoskeletal inhibitors and (b) inhibitors of acetylation, and (c) inhibitors of methylation, and also culturing the cell in the presence of agents or conditions that induce differentiation to a different cell type. The method can be useful for producing histocompatible cells for cell therapy.

This disclosure also relates to methods to obtain mammalian cells and tissues with patterns of gene expression similar to that of a developing mammalian embryo or fetus, and the use of such cells and tissues in the treatment of human disease and age-related conditions. More particularly, the disclosure includes methods for identifying, expanding in culture, and formulating mammalian pluripotent stem cells and differentiated cells that differ from cells in the adult human in their pattern of gene expression, and therefore offer unique characteristics that provide novel therapeutic strategies in the treatment of degenerative disease.

The present disclosure also provides methods for the reprogramming of animal somatic cells and methods for the derivation, formulation, and use of the resulting reprogrammed cells and engineered tissues in modalities of therapy for the prevention and treatment of disease. More specifically, the disclosure provides an improved means of reprogramming differentiated cells to an undifferentiated state, extending telomere length and therefore replicative lifespan, and accordingly producing stem cells and resulting differentiated cells of many kinds with a nuclear genotype identical to the genotype of the original differentiated cell.

The present methods may also be used to analyze the mechanisms of nuclear reprogramming and or the production of differentiated cells for use in research and therapy. The methods represent an improvement over existing techniques, such as human somatic cell nuclear transfer (SCNT), used to de-differentiate animal somatic cells into an embryonic state, thereby producing hES cells. The present disclosure provides methods to improve such existing techniques by separating cellular reprogramming into at least two, or preferably three, separate steps, utilizing in some of those steps cytoplasmic components from a donor cell source, wherein the donor source is a differentiated cell from a species different from the species of the oocyte. Using a donor cell source from a different species than the species of the oocyte eases access to reprogramming materials, the degree of successful reprogramming, and the scale-up of the process of reprogramming differentiated cells. In one embodiment, somatic differentiated cells are reprogrammed to an undifferentiated state through a novel reprogramming technique comprised of the following three steps: In the first step, designated the nuclear remodeling step, the degree of reprogramming of the somatic cell genome is increased and the problem of access to oocytes of the same species as the somatic cell is alleviated by the use of any or a combination of several novel reprogramming procedures. In all of these novel procedures, the somatic cell nucleus is remodeled to replace the components of the nuclear envelope with the components of an undifferentiated cell. Simultaneously, or at a point in time early enough to prevent the incorporation of somatic cell differentiated components into the nuclear envelope, the chromatin of said cell is reprogrammed to express genes of an undifferentiated cell. The first step is advantageous over current SCNT technology in that oocytes of the same species as the somatic cell are not required; further, an improved quality of reprogramming can be achieved.

In the second step, designated herein as the cellular reconstitution step, the nucleus, containing the remodeled nuclear envelope of step one, is either transferred to an enucleated cytoplasm of an undifferentiated embryonic cell, or is fused with a cytoplasmic bleb containing a requisite mitotic apparatus which is capable, together with the transferred nucleus, of producing a population of undifferentiated stem cells such as ES or ED-like cells capable of proliferation. The second step has the advantage over SCNT in that a large number of nuclei or chromosome clumps remodeled in step one may be simultaneously fused with cytoplasmic blebs in step two to increase the probability of obtaining reprogrammed cells capable of successfully proliferating in vitro, resulting in a large number of cultured reprogrammed cells. In the third step, colonies of cells arising from one or a number of cells resulting from step two are characterized for the extent of reprogramming and for the normality of the karyotype and colonies of a high quality are selected. While this third step is not required to successfully reprogram cells and is not necessary in some applications of the present method, such as in analyzing the molecular mechanisms of reprogramming, for many uses, such as when reprogramming cells for use in human transplantation, the inclusion of the third quality control step is preferred. Colonies of reprogrammed cells that have a normal karyotype but not a sufficient degree of reprogramming may be recycled by repeating steps 1-2 or 1-3.

In another embodiment, the nucleus is remodeled in step one by the transfer of one or numerous permeabilized or nonpermeabilized somatic cells into an oocyte of another species. The resulting remodeled nucleus or nuclei are then removed and further processed in steps two and three. In another embodiment, the genome of a somatic cell is remodeled in step one by condensation to a chromosome clump through the exposure of isolated somatic cell nuclei to an extract from mitotic cells, such as metaphase II oocytes, metaphase germ-line cells such as the EC cell line NTera-2, or of mitotic somatic cells of the same or different species. Said chromosome clumps are then further processed in steps two and three and the previous steps repeated if the cells do not show an acceptable degree of reprogramming. In another embodiment, the genome of a somatic cell is remodeled in step one by condensation to a chromosome clump through the exposure of isolated somatic cell nuclei to an extract from mitotic cells, such as metaphase II oocytes, metaphase germ-line cells such as the EC cell line NTera-2, or of mitotic somatic cells of the same or different species. Said chromosome clumps are then subsequently encapsulated in a new nuclear envelope in vitro using extracts from undifferentiated cells. The resulting remodeled nuclei are then further processed in steps two and three and the previous steps repeated if the cells do not show an acceptable degree of reprogramming. Additionally, the remodeled nuclei and cells may be used in assays to analyze the mechanisms of reprogramming.

In another embodiment, one or more factors expressed in undifferentiated cells (e.g., EC cells, ES cells, etc.) are transiently expressed or overexpressed in the undifferentiated cell extracts or cells of step 1 and/or step 2 or are added as proteins to said cell extracts. Expression of these factors may confer characteristics of an undifferentiated cell to the somatic cell and facilitate reprogramming of the somatic cell. Such factors include, for example, NANOG, SOX2, DNMT3B, CROC4, H2AFX, HIST1H2AB, HIST1H4J, HMGB2, LEFTB, MYBL2, MYC, MYCN, NANOG, OCT3/4 (POU5F1), OTX2, SALL4, TERF1, TERT, ZNF206, or any combination of the foregoing or any other factors (such as transcriptional regulators) that confer characteristics of an undifferentiated cell state. In particular, any number or combinations of the above-mentioned factors may be used the selection of which may depend upon the lineage of the somatic cell being reprogrammed or dedifferentiated. In another embodiment, the various kinds of in vitro reprogramming of step one of the present method are utilized as an in vitro model of nuclear reprogramming useful in analyzing the molecular mechanisms of reprogramming. For example, particular molecular components may be added or deleted from the extract to determine the role of certain components in reprogramming and determination of cell lineage.

In another embodiment, the various components determined to play an important role in reprogramming identified in the above assay or by other means are then correspondingly incorporated or deleted from the reprogramming extract to increase the efficiency of reprogramming in the same or cross species reprogramming protocol. Such molecules include but are not limited to human protein components, purified RNA, including miRNA from oocytes, blastomeres, morulae, ICMs, embryonic disc, ES cells, EG cells, EC cells, or other germ-line cells. The components may be added or deleted during any of steps 1-3. Particular components may be deleted by methods such as, for example, immunoprecipitation. In another embodiment, steps 1-2 are repeated as step one followed by step two, followed by step one, followed by step two, until characterization in step three demonstrates successful reprogramming of the somatic cells. In another aspect, cytoplasts from undifferentiated or germ-line cells are depleted of mitochondria to make cell lines into which donor cell mitochondria may be added before, during, or after step two to result in reprogrammed cells wherein the mitochondrial genotype as well as the nuclear genotype is identical to the donor differentiated cell. In another embodiment, undifferentiated cell factors such as, for example, SOX2, NANOG, DNMT3B, CROC4, H2AFX, HIST1H2AB, HIST1H4J, HMGB2, LEFTB, MYBL2, MYC, MYCN, NANOG, OCT3/4 (POU5F1), OTX2, SALL4, TERF1, TERT, ZNF206, are added to the cytoplasts or cytoplasmic blebs from undifferentiated or germ-line cells of step 2. In particular embodiments, two, three, four, or five of the factors are added to the cytoplasts. In other embodiments, six or more of the factors are added to the cytoplasts. In another aspect, reprogrammed cells resulting from the use of steps 1-2 or 1-3 are differentiated in a variety of in vitro, in vivo, or in vitro differentiation conditions to yield cells of any or a combination of the three primary germ layers endoderm, mesoderm, or ectoderm, including complex tissues such as tissues formed in teratomas. In certain embodiments, differentiated cell types are derived from the reprogrammed cells of the present method without the generation of an ES cell line. For example, differentiated cells may be obtained by culturing undifferentiated reprogrammed cells in the presence of at least one differentiation factor and selecting differentiated cells from the culture. Selection of differentiated cells may be based on phenotype, such as the expression of certain cell markers present on differentiated cells, or by functional assays (e.g., the ability to perform one or more functions of a particular differentiated cell type). Differentiated cells derived by the present methods include, but are not limited to, adult stem cells, pancreatic beta cells and pancreatic precursor cells. In another embodiment, the cells reprogrammed according to the present methods are genetically modified through the addition, deletion, or modification of their DNA sequence (s). Such modifications can be made by the random incorporation of an exogenous vector, by gene targeting, or through the use of artificial chromosomes. In another embodiment of the present methods, the nucleus being remodeled in step one is modified by the addition of extracts from cells such as, for example, DT40, known to have a high level of homologous recombination. The addition of DNA targeting constructs and the extracts from cells permissive for a high level of homologous recombination will then yield cells after reconstitution in step 2 and screening in step 3 that have a desired genetic modification.

Another embodiment is a business model for commercializing cells produced from the use of said method. The business model includes the transfer of human somatic differentiated cells to regional centers where the reprogramming steps 1, 2, 1-2 or 1-3 are performed. In another embodiment, the differentiated somatic cells or the reprogrammed cells resulting from the application of steps 1, 2, 1-2 or 1-3 are cryopreserved and banked for future use. In another embodiment, the reprogrammed cells resulting from the application of steps 1, 2, 1-2, or 1-3 are shipped to health care facilities where they are differentiated into medically useful cell types for use in research and transplantation. In another embodiment, kits containing ingredients useful in performing the activities of steps 1, 2, or 3 are shipped to research, biomedical, or health care facilities where they are used to reprogram differentiated cells into cell types for use in research and transplantation.

In another embodiment, the reprogrammed cells resulting from the application of steps 1, 2, 1-2, or 1-3 are shipped to health care facilities after having been differentiated into a useful composition of cell types.

Other features and advantages of the invention will be apparent from the following description and from the claims, though embodiments may also achieve fewer than all of the advantages or different advantages than those exemplified above.

### BRIEF DESCRIPTION OF THE DRAWINGS

FIG. 1 illustrates the pTAT-HA vector used for cloning and bacterial expression of certain reprogramming proteins.

FIG. 2 shows the nickel column-purified TAT-hOCT4 and TAT-hNanog constructs on stained electrophoresis gels.

FIG. 3 shows the nickel column-purified TAT-Klf4, TAT-Sox2 and TAT-cMyc on stained electrophoresis gels.

FIG. 4 shows the decreased intensity of Alkaline Phosphatase staining after human ES cells were treated with TAT-hOct4 fusion protein.

FIG. 5 illustrates the pSecTag2B vector used for mammalian expression of certain reprogramming proteins.

FIG. 6 shows the pSecTag2B vector multiple cloning site.

FIG. 7 shows the Oct4 and Nanog fusion proteins (immunopurified from 293 cells) on stained electrophoresis gels.

FIG. 8 shows entry of the Oct4 and Nanog fusion proteins into neonatal human dermal fibroblasts, 36 h after protein transfection.

FIG. 9 Uptake of fluorescent Rhodamine-Albumin by SLO permeabilized 293T cells using optimized protocols. Human 293T cells were permeabilized using SLO in the presence of Rhodamine-labeled Albumin. Left panel: bright field microscopy images; Right panel: fluorescence microscopy view of the same field.

FIG. 10. Characterization of undifferentiated hES cell cultures used to generate whole cell extracts. Cultures of hES cell line ACT-4 were examined by (a) phase contrast microscopy; (b) alkaline phosphatase activity assay; and immunofluorescence for the expression of hES cell markers (c) Oct-4 (e) SSEA-3, and (f) Tra-1-81. Panel (d) depicts the DAPI stain for nuclei of the same field as stained for Oct-4 in (c).

FIG. 11. Morphology of cell colonies obtained after reprogramming incubations using hES cell extracts and permeabilized 293T cells. 293T cells were permeabilized and incubated with either control 293T extracts (left column, FIGS. 11A and 11C) or hES cell extracts (right column, FIGS. 11B and 11D) before plating on MEF feeder layers in hES cell culture conditions. Colonies obtained were examined by phase contrast microscopy. Results shown from two experiments (first experiment, 11A-B; second experiment, 11C-D). Magnification: 40×.

FIGS. 12 and 13: Cells with neuronal morphology produced by treating bovine fetal fibroblasts CB at 2.5-7.5 μg/m and culturing them under conditions that induce neural differentiation. The cells in FIG. 12 were observed with phase contrast microscopy; those in FIG. 14 were observed by DIC. FIG. 12: (A) Control, (B) 2.5 μg/ml, (C) 5.0,μg/ml, (D) 7.5 μg/ml

FIGS. 14 and 15: Cells with neuronal morphology produced by treating bovine adult fibroblasts CB at 10.0 μg/m and culturing them under conditions that induce neural differentiation.

FIG. 16: Cells with neuronal morphology produced by treating human fetal fibroblasts CB at 5.0 μg/m and culturing them under conditions that induce neural differentiation. (A) Control, (B) 2.5 μg/ml, (C) 5.0 μg/ml, (D) 7.5 μg/ml

FIG. 17: Photographs showing the presence of neural-specific markers nestin and Tuj1 in human fetal fibroblasts treated with CB at 5.0 μg/m and cultured under conditions that induce neural differentiation.

FIG. 18 shows the remodeling of multiple somatic cell nuclei within one oocyte, the subsequent lysing of the oocyte to retrieve remodeled nuclei, and their fusion with ES cell cytoplasmic blebs to yield ES cell lines.

FIG. 19 shows a diagram displaying the modification of isolated chromosomes, chromatin, or nuclei in vitro. Purified recombinase or cell free extract is shown as spheres.

### DETAILED DESCRIPTION

Reprogramming Agents

As noted above, “reprogramming” herein refers to methods whereby a desired recipient or donor cell or a recipient cell nucleus or chromosomal DNA thereof is converted into a less differentiated cell or nucleus (e.g., a dedifferentiated cell comprising said reprogrammed cell or nucleus such as an iPS or ESC or adult stem cell) or a cell of a different type or lineage by introducing into or incubating same with a composition containing or encoding one or more reprogramming factors. Successful dedifferentiation and transdifferentiation of somatic cells and somatic cell nuclei induced to dedifferentiate or transdifferentiate by primitive cell cytoplasm has confirmed the existence of substances or factors therein capable of promoting or triggering cell or cell nucleus reprogramming. Certain gene products including Oct4 that are expressed in primitive cells have been shown to be sufficient to cause dedifferentiation of somatic cells and somatic cell nuclei when these gene products are present in sufficient amount(s) and duration, e.g., wherein expression of these gene products is induced by viral transformation. These results have confirmed that the cytoplasm of cells in early or primitive states of development contain genes that are sufficient to trigger or promote cell dedifferentiation.

Exemplary reprogramming agents that can be used in the methods described herein include reprogramming polypeptides and small molecules, and optionally include facilitating agents. Reprogramming polypeptides include Oct4, Sox2, Nanog, c-Myc, Klf4, and Lin28 as well as functional fragments, variants and fusions containing any of the foregoing. Genes that encode these and other reprogramming polypeptides are shown in Tables 1 and 2, respectively. These reprogramming polypeptides may be used individually or in combinations.

In one exemplary embodiment, combinations of different reprogramming polypeptides may be tested by the methods described herein to identify those of which alone or in combination result in successful reprogramming of a particular donor or recipient cell or cell nucleus. Though the number of possible combinations is quite large, pooling methods may be used to greatly reduced the effort required to identify effective combinations. For example, Takahashi and Yamanaka, supra, used retrovirus cocktails containing up to 24 genes to dedifferentiate somatic cells; subsequently, “leave one out” experiments permitted identification of an effective group of genes. Similar methods can be used to identify operative recombinant reprogramming polypeptides and cocktail thereof. A complementary approach employs similar retroviral methods, but takes advantage of the heterogeneity of transformed cell populations by testing the resulting dedifferentiated cells to identify which the combinations of retroviruses that actually integrated into each dedifferentiated cell line. Methods known in the art, particularly high-throughput methods such as microarrays and PCR (using candidate gene-specific sequences and/or barcodes included in the retroviral constructs), can be used to identify combinations of integrated constructs that gave rise to dedifferentiated cells. For example, virus-based methods may be used to identify effective combinations of reprogramming genes, and effective combinations may then be used to reprogram cells using methods that do not cause genome sequence modification, such as by contact with the polypeptides these genes encode.

Using these methods different types of somatic cells and cells of different species may be most effectively reprogrammed by one or more reprogramming agents and combinations thereof. The methods described herein will allow such species-specific and cell type-specific combinations of reprogramming agents to be readily identified. These methods will, while identifying operative reprogramming factor combinations may yield different results dependent on the particular cell being reprogrammed. For example because certain recipient cells, such as adult stem cells may endogenously express one or more reprogramming polypeptides (in sufficient levels for reprogramming to be effectuated without the need for that reprogramming polypeptide to be exogenously added) these cells may be reprogrammed (e.g., into iPC's) using few (e.g., a single reprogramming factor). By contrast reprogramming of cells which do not endogenously express any reprogramming factors may require the use of a cocktail containing several reprogramming factors. For example, neural progenitors and astroglia have been shown to endogenously express Sox2 (Komitova and Eriksson, Neurosci Lett. 2004 Oct. 7; 369(1):24-7; Ellis et al., Dev Neurosci. 2004 March-August; 26(2-4):148-65; Avilion et al., Genes Dev. 2003 Jan. 1; 17(1):126-40; D'Amour and Gage, Proc Natl Acad Sci USA. 2003 Sep. 30; 100 Suppl 1:11866-72; Miyagi et al., Mol Cell Biol. 2004 May; 24(10):4207-20) and accordingly are expected to be effectively reprogrammed without addition of exogenous Sox2.

Other exemplary reprogramming agents of the present methods include agents that cause expression of candidate reprogramming polypeptides. These include traditional methods of inducing gene expression, such as mRNAs, retroviruses, as well as small molecules that may induce a cell to express reprogramming genes. For example, a suppressor of reprogramming gene expression may be inhibited using siRNA techniques. These agents may be identified by screening methods, preferably high-through screening methods, that are well known in the art.

In certain exemplary embodiments the reprogramming agent can include one or more agents that can facilitate reprogramming (“reprogramming facilitating agents”). Exemplary reprogramming facilitating agents help facilitate epigenetic changes that occur during reprogramming. Exemplary reprogramming facilitating agents include deacetylase inhibitors and DNA methylation inhibitors, such as through RNAi targeting genes involved in histone deacetylation or DNA methylation. Deacetylase inhibitors also include trichostatin A (Yoshida et al., Bioessays. 1995 May; 17(5):423-30), vorinostat (Zolinza, available from Merck & Co., Inc.), and valproic acid. DNA methylation inhibitors include methyltransferase inhibitors such as 5-aza-cytidine; Boukamp, Semin Cell Biol. 1995 June; 6(3):157-63) and 5-aza-2′-deoxycytidine.

TABLE 1
Mouse candidate reprogramming genes

| Gene | Accession No. (Mouse) |
|---|---|
| c-Myc | NM_010849 |
| Dnmt31 | NM_019448 |
| Dppa2 | NM_028615 |
| Dppa3 (Stella) | NM_139218 |
| Dppa4 | NM_028610 |
| Dppa5 (Esg1) | NM_025274 |
| Ecat1 | AB211060 |
| Ecat8 | AB211061 |
| ERas | NM_181548 |
| Fbxo15 | NM_015798 |
| Fthl17 | NM_031261 |
| Gdf3 | NM_008108 |
| Grb2 | NM_008163 |
| Klf4 | NM_010637 |
| Nanog | AB093574 |
| Oct¾ (Pou5f1) | NM_013633 |
| Rex1 (Zfp42) | NM_009556 |
| Sall4 | NM_175303 |
| Sox15 | NM_009235 |
| Sox2 | NM_011443 |
| Stat3 | NM_213659 |
| Tcl1 | NM_009337 |
| Utf1 | NM_009482 |
| β-catenin | NM_007614 |

TABLE 2
Human candidate reprogramming genes.

| Gene | Accession No. (Human) |
|---|---|
| ACRBP | NM_032489 |
| AKT | NM_005163 |
| BARX1 | NM_021570 |
| BCL2 | NM_000633 |
| C10orf96 | NM_198515 |
| C14orf115 | NM_018228 |
| C9orf135 | NM_001010940 |
| CCNF | NM_001761 |
| CER1 | NM_005454 |
| CLDN6 | NM_021195 |
| CROC4 | NM_006365 |
| CTSL2 | NM_001333 |
| DDX25 | NM_013264 |
| DKFZp761P0423 | XM_291277 |
| DNMT3B isoform 1 | NM_006892 |
| DNMT3B isoform 2 | NM_175848 |
| DNMT3B isoform 3 | NM_175849 |
| DNMT3B isoform 6 | NM_175850 |
| DNMT3L | NM_013369 |
| DPPA2 | NM_138815 |
| DPPA3 | NM_199286 |
| DPPA4 | NM_018189 |
| DPPA5 | NM_001025290 |
| ECAT1 | NM_001017361 |
| ECAT11 | NM_019079 |
| ECAT8 | XM_117117 |
| EMID2 | NM_133457 |
| FLJ35934 | NM_207453 |
| FLJ40504 | NM_173624 |
| FLJ43965 | NM_207406 |
| FOXD3 | NM_012183 |
| FOXH1 | NM_003923 |
| GAP43 | NM_002045 |
| GBX2 | NM_001485 |
| GDF3 | NM_020634 |
| GPC2 | NM_152742 |
| GPR176 | NM_007223 |
| GPR23 | NM_005296 |
| H2AFX | NM_002105 |
| HES3 | NM_001024598 |
| HESX1 | NM_003865 |
| HHEX | NM_002729 |
| HIST1H2AB | NM_003513 |
| HIST1H4J | NM_021968 |
| HMGB2 | NM_002129 |
| HRASLS5 | NM_054108 |
| hsa-miR-106a | MI0000113 |
| hsa-miR-107 | MI0000114 |
| hsa-miR-141 | MI0000457 |
| hsa-miR-183 | MI0000273 |
| hsa-miR-187 | MI0000274 |
| hsa-miR-18a | MI0000072 |
| hsa-miR-18b | MI0001518 |
| hsa-miR-203 | MI0000283 |
| hsa-miR-20b | MI0001519 |
| hsa-miR-211 | MI0000287 |
| hsa-miR-217 | MI0000293 |
| hsa-miR-218-1 | MI0000294 |
| hsa-miR-218-2 | MI0000295 |
| hsa-miR-302a | MI0000738 |
| hsa-miR-302c | MI0000773 |
| hsa-miR-302d | MI0000774 |
| hsa-miR-330 | MI0000803 |
| hsa-miR-363 | MI0000764 |
| hsa-miR-367 | MI0000775 |
| hsa-miR-371 | MI0000779 |
| hsa-miR-372 | MI0000780 |
| hsa-miR-373 | MI0000781 |
| hsa-miR-496 | MI0003136 |
| hsa-miR-508 | MI0003195 |
| hsa-miR-512-3p | MIMAT0002823 |
| hsa-miR-512-5p | MIMAT0002822 |
| hsa-miR-515-3p | MIMAT0002827 |
| hsa-miR-515-5p | MIMAT0002826 |
| hsa-miR-516-5p | MI0003172 |
| hsa-miR-517 | MIMAT0002851 |
| hsa-miR-517a | MI0003161 |
| hsa-miR-518b | MI0003156 |
| hsa-miR-518c | MI0003159 |
| hsa-miR-518e | MI0003169 |
| hsa-miR-519e | MI0003145 |
| hsa-miR-520a | MI0003149 |
| hsa-miR-520b | MI0003155 |
| hsa-miR-520e | MI0003143 |
| hsa-miR-520g | MI0003166 |
| hsa-miR-520h | MI0003175 |
| hsa-miR-523 | MI0003153 |
| hsa-miR-524 | MI0003160 |
| hsa-miR-525 | MI0003152 |
| hsa-miR-526a-1 | MI0003157 |
| hsa-miR-526a-2 | MI0003168 |
| hsa-miR-96 | MI0000098 |
| LEFTB | NM_020997 |
| LHX1 | NM_005568 |
| LHX5 | NM_022363 |
| LHX6 | NM_014368 |
| LIN28 | NM_024674 |
| LIN28B | NM_001004317 |
| LIN41 | NM_001039111 |
| LOC138255 | NM_001010940 |
| LOC389023 | BC032913 |
| LOC643401 | BC039509 |
| MDK | NM_001012334 |
| MGC27016 | NM_144979 |
| MIRH1 | XM_931068 |
| MIXL1 | NM_031944 |
| Mybl2 | NM_002466 |
| MYC | NM_002467 |
| MYCN | NM_005378 |
| NANOG | NM_024865 |
| NFIX | NM_002501 |
| NHLH2 | NM_005599 |
| NODAL | NM_018055 |
| NPM2 | NM_182795 |
| NPM3 | NM_006993 |
| NR0B1 | NM_000475 |
| NUT | NM_175741 |
| OCT¾ (POU5F1) | NM_002701 |
| OCT6 (POU3F1) | NM_002699 |
| OTX2 | NM_172337 |
| PHB | NM_002634 |
| PHC1 | NM_004426 |
| PIWIL2 | NM_018068 |
| POU3F2 | NM_005604 |
| POU6F1 | NM_002702 |
| PRDM14 | NM_024504 |
| PRTG | NM_173814 |
| PUNC | NM_004884 |
| RABGAP1L | NM_014857 |
| RKHD3 | NM_032246 |
| RPGRIP1 | NM_020366 |
| SALL1 | NM_002968 |
| SALL2 | NM_005407 |
| SALL3 | NM_171999 |
| SALL4 | NM_020436 |
| SCGB3A2 | NM_054023 |
| SLITRK1 | NM_052910 |
| SOX10 | NM_006941 |
| SOX11 | NM_003108 |
| Sox15 | NM_006942 |
| SOX2 | NM_003106 |
| SOX21 | NM_007084 |
| SP8 | NM_198956 |
| SPANXC | NM_022661 |
| SYT6 | NM_205848 |
| T (brachyury homolog) | NM_003181 |
| TCL1A | NM_021966 |
| TDGF1 | NM_003212 |
| TDRD5 | NM_173533 |
| TERF1 | NM_003218 |
| TERF1 | NM_017489 |
| TERT | NM_198254 |
| TGIF | NM_003244 |
| TSGA10IP | NM_152762 |
| UNC5D | NM_080872 |
| USP44 | NM_032147 |
| UTF1 | NM_003577 |
| VENTX2 | NM_014468 |
| ZFP42 | NM_174900 |
| ZIC2 | NM_007129 |
| ZIC3 | NM_003413 |
| ZIC5 | NM_033132 |
| ZNF124 | NM_003431 |
| ZNF206 | NM_032805 |
| ZNF342 | NM_145288 |
| ZNF677 | NM_182609 |
| ZNF738 | BC034499 |

Methods for Introducing Reprogramming Agents into Cells or Using Same to Reprogram Cell Nuclei

Numerous methods are known to one of skill in the art for effecting transport and delivery of a desired polypeptides or nucleic acids or small molecules into a recipient cell or cell nucleus and may be used to effectively deliver reprogramming agents into cells or cell nuclei These methods include by way of example electroporation, microinjection, liposomes, cationic lipids, cell permeabilization, incubation or contacting with donor cell cytoplasm or cytoplasmic blebs and/or linkage thereof to one or more protein transduction domains (PTD) or nuclear translocation domain or nuclear localization signals moieties (NTD or NTM or NTS moieties). Examples of such moieties which facilitate nuclear delivery of substituents attached thereto include by way of example SV40 T-antigen localization signal, the C-terminus of apoptin, acridine nuclear localization signal, polyargine (arg11), s4 13-PV, adenovirus hexon protein, PV-S4(13), RR-S4(13), et al. NLSs are generally short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-.beta. nuclear localization signal (ARRRRP); NF kappa B p50 (EEVQRKRQKL; Ghosh et al., Cell 62:1019 (1990); NF kappa B p65 (EEKRKRTYE; Nolan et al, Cell 64:961 (1991); and others (see, for example, Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann. Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

For example, electroporation may be used to introduce DNA into mammalian cells (Neumann, E. et al. (1982) EMBO J. 1, 841-845), as well as plant and bacterial cells, and may also be used to introduce proteins (Marrero, M. B. et al. (1995) J. Biol. Chem. 270, 15734-15738; Nolkrantz, K. et al. (2002) Anal. Chem. 74, 4300-4305; Rui, M. et al. (2002) Life Sci. 71, 1771-1778). Cells (such as the cells of this disclosure) can be suspended in a buffered solution containing the protein, DNA, or other molecule of interest are placed in a pulsed electrical field. Briefly, high-voltage electric pulses result in the formation of small (nanometer-sized) pores in the cell membrane. Molecules enter the cell via these small pores or during the process of membrane reorganization as the pores close and the cell returns to its normal state. The efficiency of delivery is dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines remain refractory even to electroporation unless partially activated.

Microinjection can be used to introduce femtoliter volumes containing molecules of interest directly into the nucleus of a cell. It has been used to introduce DNA directly into the nucleus of a cell (Capecchi, M. R. (1980) Cell 22, 470-488) where it was integrated directly into the host cell genome, thus creating an established cell line bearing the sequence of interest. Proteins such as antibodies (Abarzua, P. et al. (1995) Cancer Res. 55, 3490-3494; Theiss, C. and Meller, K. (2002) Exp. Cell Res. 281, 197-204) and mutant proteins (Naryanan, A. et al. (2003) J. Cell Sci. 116, 177-186) can also be directly delivered into cells via microinjection. Other molecules of interest, including RNA, episomal DNA, small molecules, proteins, etc., can also be introduced into cells by similar methods. Microinjection has the advantage of introducing macromolecules directly into the cell, thereby bypassing exposure to potentially undesirable cellular compartments such as low-pH endosomes. Microinjection can be performed manually and using semi-automated and fully automated microinjection systems, e.g., as described in: Matsuoka et al., Journal of Biotechnology, Volume 116, Issue 2, 16 Mar. 2005, Pages 185-194; Zhang and Yu, Current Opinion in Biotechnology, Volume 19, Issue 5, October 2008, Pages 506-510; Wang et al., PLoS One. 2007 Sep. 12; 2(9):e862; Ito et al., U.S. PGPub. No. 2008/0299647; Ito et al., U.S. PGPub. No. 2008/0268540; Japanese Patent No. 2624719; Ando et al., U.S. PGPub. No. 2008/0002868; Myiawaki et al., U.S. PGPub. No. 2007/0087436.

Liposomes can also be used to introduce molecules into cells. Liposomes have been used to deliver oligonucleotides, DNA (gene) constructs and small drug molecules into cells (Zabner, J. et al. (1995) J. Biol. Chem. 270, 18997-19007; Felgner, P. L. et al. (1987) Proc. Natl. Acad. Sci. USA 84, 7413-7417). Certain lipids, when placed in an aqueous solution and sonicated, form closed vesicles consisting of a circularized lipid bilayer surrounding an aqueous compartment. These vesicles or liposomes can be formed in a solution containing the molecule to be delivered. In addition to encapsulating DNA in an aqueous solution, cationic liposomes can spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Felgner, J. H. et al. (1994) J. Biol. Chem. 269, 2550-2561). The cationic liposome strategy has also been applied successfully to protein delivery (Zelphati, O. et al. (2001) J. Biol. Chem. 276, 35103-35110). Because proteins are more heterogeneous than DNA, the physical characteristics of the protein such as its charge and hydrophobicity will influence the extent of its interaction with the cationic lipids.

Cationic lipid complexes can also be used to introduce molecules into cells. For example, the Pro-Ject Protein Transfection Reagent may be used. The Pro-Ject Protein Transfection Reagent utilizes a cationic lipid formulation that is noncytotoxic and is capable of delivering a variety of proteins into numerous cell types. The molecule to be introduced is mixed with the liposome reagent and is overlayed onto cultured cells. The liposome:molecule complex is believed to facilitate entry into cells via fusion with the cell membrane or internalization via an endosome. The molecule of interest is released from the complex into the cytoplasm free of lipids (Zelphati, O. and Szoka, Jr., F. C. (1996) Proc. Natl. Acad. Sci. USA 93, 11493-11498) and escaping lysosomal degradation. The noncovalent nature of these complexes is a major advantage of the liposome strategy as the delivered protein is not modified and therefore is less likely to lose its activity. Other cationic lipid systems used for introduction of molecules into cells include PULSin™ (Polyplus Transfection, distributed by Genesee Scientific, 8430 Juniper Creek Lane, San Diego, Calif. 92126) and SAINT-PhD (Synvolux Therapeutics B.V., L. J. Zielstraweg 1, 9713 GX Groningen, The Netherlands). PULSin™ contains a proprietary cationic amphiphile molecule that forms non-covalent complexes with proteins and antibodies. Complexes are believed to be internalized via anionic cell-adhesion receptors and are released into the cytoplasm where they disassemble. The process is non-toxic and delivers functional proteins. SAINT-PhD consists of a proprietary cationic pyridinium amphiphile and a helper lipid. Upon mixture of SAINT-PhD with the protein a particle of approximately 200 nm in diameter is formed. In this particle the protein is enwrapped by at least one bilayer of lipids. Furthermore, in the complex formed only non-covalent interactions are present between SAINT-PhD and the protein. The cationic amphiphiles on the surface of the particle have high affinity for the negatively charged cell surface. Upon fusion or entrapment of the particle the protein is released into the cytoplasm of the cell. The proteins delivered by SAINT-PhD are functional and unmodified.

Molecules can also be introduced into cells or nuclei through cell or nuclear membrane permeabilization, for example, by use of digitonin or Streptolysin O, Streptolysin O can form pores up to the size of 35 nm in the plasma membrane of mammalian cells, which is generally lethal to the cell (Bhakdi et al., Adv Exp Med Biol. 1985; 184:3-21; Bhakdi et al., Infect Immun. 1985 January; 47(1):52-60; Walev et al., Proc Natl Acad Sci USA. 2001 Mar. 13; 98(6):3185-90; Walev et al., FASEB J. 2002 February; 16(2):237-9). However, transient low-dosage treatment with Streptolysin O in the absence of calcium ions allows the transient formation of membrane pores that are large enough to allow the passive diffusion of proteins. These pores are subsequently repaired upon addition of calcium ions, resulting in viable cells. Streptolysin O has been used to introduce molecules including anti-sense oligonucleotides and functional proteins into a cell (Fawcett et al., Exp Physiol. 1998 May; 83(3):293-303; Walev et al., supra). In one embodiment, Streptolysin O can be used to permeabilize the cellular membrane to allow the cells to be loaded with cellular extracts of another cell type. For permeabilization with Streptolysin O, cells are typically incubated in Streptolysin O solution (see, for example, Maghazachi et al., FASEB J. 1997 August; 11(10):765-74) for 15-30 minutes at room temperature. For digitonin permeabilization, cells are suspended in culture medium containing digitonin at a concentration of approximately 0.001-0.1% and incubated on ice for 10 minutes. After permeabilization, the cells are typically washed by centrifugation at 400×g for 10 minutes. Typically, this washing step is repeated twice by resuspension and sedimentation in PBS. Cells are typically kept in PBS at room temperature until use. Alternatively, the cells can be permeabilized while placed on coverslips to minimize the handling of the cells and to eliminate the centrifugation of the cells, which in some instances can improve the viability of the cells. The permeabilized cells are then contacted with the desired substances (e.g., cell extract, purified protein, etc.). After the procedure, the cellular membrane of cells treated with Streptolysin O can be resealed in the presence of calcium.

Molecules can also be introduced into cells or cell nuclei by linkage to a protein transduction domain (PTD) or nuclear translocation domain or nuclear localization signal such as those already mentioned. For example, a protein may be expressed as a fusion protein that includes a PTD or NLS. Additionally, a molecule to be introduced into a cell may be covalently or noncovalently linked to a PTD or NLS using other means known in the art, e.g., using a chemical linker, avidin-biotin linkage, streptavidin-biotin linkage, Protein A/Fc linkage, Protein G/Fc linkage, etc. Exemplary PTDs that may be used for introduction of molecules of interest into cells are described, under the heading “Fusion Proteins,” infra. Multiple PTDs (which may be the same or different) may be linked to a molecule to be introduced into a cell.

Another means of introducing molecules into a recipient cell or nucleus comprises the introduction of or contacting with cytoplasm blebs derived from a donor cell.

The recipient cell can be of any species and may be heterologous to the donor cell, e.g., amphibian, mammalian, avian, with mammalian cells being preferred. Especially preferred recipient cells include human and other primate cells, e.g., chimpanzee, cynomolgus monkey, baboon, other Old World monkey cells, caprine, equine, porcine, ovine, and other ungulates, murine, canine, feline, and other mammalian species.

Exemplary methods of introducing donor cell cytoplasm into a recipient cell include microinjection, contacting donor cells with liposomal encapsulated cytoplasm, and enucleating the donor cell and incubating the recipient cell with a donor cell cytoplasmic extract. For example, this can be effected by microsurgically removing part or all of the cytoplasm of a donor cell with a micropipette and microinjecting such cytoplasm into that of a recipient cell. It may also be desirable to remove cytoplasm from the recipient cell prior to such introduction. Such removal may be accomplished by well known microsurgical methods. Alternatively, the cytoplasm and/or telomerase or telomerase DNA can be introduced using a liposomal delivery system.

In one embodiment, a polypeptide can be provided in the recipient cell media by being produced and secreted by engineered cells. For example, feeder cells may be engineered to express and secrete one or more desired reprogramming polypeptides. Optionally, engineered cells are physically separated from the recipient cells, e.g., by a selective barrier which may contain pores that allow diffusion of the reprogramming polypeptides but are too small for cells to pass through. Secretion of the reprogramming polypeptides may be effected through means known in the art, such as by fusion to a secretion signal. For example, a protein may be fused to or engineered to comprise a signal peptide, or a hydrophobic sequence that facilitates export and secretion of the protein. Whatever method is used to provide reprogramming proteins and other reprogramming agents in the cell media, those reprogramming agents can then be introduced into the recipient cells by any of the foregoing methods, preferably by linkage to a protein transduction domain, cell permeabilization, and/or addition of cationic lipids.

Treatment of Disease

Many diseases resulting from the dysfunction of cells may be amenable to treatment by the administration of hES-derived cells of various differentiated types. These include diseases of cardiac, neurological, endocrinological, vascular, retinal, dermatological, and muscular-skeletal systems, and other diseases.

Transforming a patient's own cells into a desired cell type that needs replacement, reprogramming will permit the generation of autologous, genetically matched cells that would not be subject to immune rejection on transplantation. Additionally, stem cell lines created according to the methods described herein can be a source of cells for transplantation.

In one embodiment, a stem cell is prepared from a patient's relative, such as a histocompatible relative. For example, for treatment of a patient having a genetic disorder, a stem cell may be prepared from a transplant-compatible relative who does not have the genetic disorder.

Preferably the cells are histocompatible with the individual recipient, such that the undesirable use of immunosuppression is decreased or eliminated. For example, histocompatible cells may be obtained from the patient, from a donor related to the patient, or an unrelated donor. Optionally the cells are genetically modified so alter their histocompatibility profile, such that they are more compatible with the patient.

A “bank” of different stem cell lines can be created by the methods herein, and can provide sources of cells for therapeutic transplant that are highly histocompatible with human or non-human patients in need of cell transplants. For example, a stem cell line may be established from a patient, a relative of the patient, or an unrelated individual. In a more specific embodiment a bank of different stem cell lines, such as different types of adult stem cell lines may be produced for potential use in cell therapies or transplantation therapy as the need may arise. Thus, an object of the present disclosure to prepare a collection of totipotent, nearly totipotent, and/or pluripotent stem cell lines that can be used for therapeutic transplant. Certain of the stem cell lines are homozygous for at least one histocompatibility antigen, which is particularly desirable to increase the number of individuals histocompatible with a given line. In addition these cells may be genetically modified before, during or after reprogramming so as to eliminate a genetic defect that is correlated to a specific disease so as to preclude disease relapse in transplantation therapies using cells produced using the subject reprogramming methods such as pancreatic cells or bone marrow cells.

Optionally, a stem cell line will be induced to differentiate into one or more desired cell types prior to introduction into a patient. Among differentiation derivatives that can be produced in vitro are such sought-after cells as cardiomyocytes, neurons, oligodendrocytes, retinal pigment epithelium, insulin-producing cells and others. Such cells and tissues, if robustly produced from ES cells, would satisfy an unmet medical need for tissue and organ repair and could be generated to decrease the risk of immune rejection either through banking a variety of genetically diverse cell lines or via patient-specific nuclear transfer technology.

The cells may be used in various methods known in the art, including being injected into a patient, grown on a scaffold and surgically implanted, directly applied to the site of an injury, etc.

For example, neurodegenerative disease frequently include neuronal cell loss, and, because of the absence of endogenous repopulation, effective recovery of function is either extremely limited or absent. Reprogrammed cells of the present disclosure may be used as a source for cell-based therapies for neurodegenerative disease diseases, including Parkinson's disease, Amyotrophic Lateral Sclerosis, Multiple System Atrophy, Tay-Sachs Disease, Alzheimer's disease, Alexander's disease, Alper's disease, Ataxia telangiectasia, Batten disease, Bovine spongiform encephalopathy (BSE), Canavan disease, Cerebral palsy, Cockayne syndrome, Corticobasal degeneration, Creutzfeldt-Jakob disease, Familial Fatal Insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spinocerebellar ataxia, Spinal muscular atrophy, Steele-Richardson-Olszewski disease, Tabes dorsalis, and Toxic encephalopathy.

Also, the subject methods may be used for the production of autologous grafts, e.g., skin grafts, which can be used in the case of tissue injury or elective surgery.

Yet another application of the present application is for treating the effects of chronologic and UV-induced aging on the skin. As skin ages, various physical changes may be manifested including discoloration, loss of elasticity, loss of radiance, and the appearance of fine lines and wrinkles. It is anticipated that such effects of aging may be alleviated or even reversed by topical application of reprogramming factor-containing compositions. For example, reprogramming factor-containing compositions, optionally further including telomerase or a telomerase DNA construct, can be packaged in liposomes to facilitate internalization into skin cells upon topical application. Also, it may be advantageous to include in such compositions compounds that facilitate absorption into the skin, e.g., DMSO. These compositions may be topically applied to areas of the skin wherein the effects of aging are most pronounced, e.g., the skin around the eyes, the neck and the hands.

The present disclosure also provides methods for alleviating the effects of aging. Just as mammalian cells have a finite life-span in tissue culture, they similarly have a finite life-span in vivo. This finite life-span is hypothesized to explain at least some of the undesired effects of aging (including decreased immune-system function). The present disclosure provides methods to alleviate the effects of aging by providing methods of reprogramming cells in situ through contact with reprogramming factors. Additionally, the present disclosure provides methods to alleviate the effects of aging by providing a source of rejuvenated cells, e.g., stem cells or differentiated cells resulting from reprogramming. For example, stem cells may be used to produce differentiated cell types in tissue culture and these cells can then be introduced into the individual. This can be used, e.g., to rejuvenate the immune system of an individual. Such rejuvenation should be useful in the treatment of diseases thought to be of immune origin, e.g., some cancers, age-associated decrease of immune function, etc.

Genetically Modified Cells

Another significant application of the present disclosure is for gene therapy. To date, many different genes of significant therapeutic importance have been identified and cloned. Moreover, methods for stably introducing such DNAs into desired cells, e.g., mammalian cells and, more preferably, human somatic cell types, are well known. Also, methods for effecting site-specific insertion of desired DNAs via homologous recombination are well known in the art.

The present methods will make it possible to produce cloned and chimeric animals having complex genetic modifications. This will be especially advantageous for the production of animal models for human diseases. Also, the present methods will be beneficial in situations wherein the expression of a desired gene product or phenotype is dependent upon the expression of different DNA sequences, or for gene research involving the interrelated effects of different genes on one another. Moreover, it is anticipated that the present methods will become very important as the interrelated effects of the expression of different genes on others becomes more understood.

Another exemplary genetic modification is introduction of a conditional “suicide gene” such as a suicide gene under a conditional promoter. For example, if for any reason the transplanted cells react in a in a way that can harm the recipient, expression of the suicide genes can be induced to kill some or all of the transplanted cells. Use of inducible suicide genes in this manner is known in the art. Suitable suicide genes include genes encoding HSV thymidine kinase and cytodine deaminase, with which cell death is induced by gancyclovir and 5-fluorocytosine, respectively. A suicide gene may also be placed under the control of a lineage-specific promoter, such that cells in which that promoter is activated are eliminated.

Exemplary genetic modifications include modifications that change a cell's histocompatibility profile, for example, by alteration of one or more HLA genes, such as by allele replacement or deletion. For example, such methods may be used to generate a “bank” of cell lines suitable for transplant into patients having different histocompatibility profiles.

Other exemplary genetic modifications decrease immune rejection responses, such as modifications that cause expression proteins that inhibit immune rejection responses such as CD40-L (CD154 or gp139), modifications that prevent generation of an antigen that can trigger an immune rejection response, e.g. a glycosylated antigen expressed by porcine or other animal cells.

Exemplary genetic modifications include replacement of a disease-associated or disease-susceptible genomic sequence with a wild-type or disease-resistant sequence. For example, introduction of a gene or replacement of alleles of a gene contained in the cell line that provides resistance to disease (e.g., an HIV-resistant allele of CCR5, such as the CCR5 delta 32 allele; a cancer-resistant allele of an oncogene or tumor suppressor gene). Another exemplary genetic modification is introduction of increased copies of the tumor suppressor gene p53, which has been shown to decrease cancer incidence and improve health-span in mice (Garcia-Cao et al., EMBO J. 2002 Nov. 15; 21(22):6225-35). Other exemplary genetic modifications include those that eliminate mutations correlated to neoplastic, autoimmune, or other genetic diseases such as cystic fibrosis, sickle cell anemia, breast cancer, prostate cancer and the like. Another exemplary genetic modification is introduction of increased copy number of the DSCR1 gene and/or the Dyrk1a, which are genes located on human chromosome 21 that have been implicated in the greatly decreased cancer incidence in individuals affected with Down's Syndrome (Baek et al., Down's syndrome suppression of tumor growth and the role of the calcineurin inhibitor DSCR1. Nature advance online publication 20 May 2009|doi:10.1038/nature08062). Certain embodiments include an increased copy number of a tumor suppressor gene (such as p53 or Rb). Other embodiments include increased copy number and/or modifications that result in increased expression of certain genes that are expected to promote health and/or fight disease including genes involved in DNA repair, antioxidant defense gene (e.g., a superoxide dismutase such as SOD1, SOD2, SOD3, a catalase), genes involved in DNA repair or chromosome maintenance, telomerase genes, etc.

Other embodiments include introduction of exogenous genes expected to provide health benefits to a cell transplant recipient. For example, certain embodiments can include introduction of genes encoding enzymes capable of selectively degrading pathogenic material that accumulates with age and has been implicated in age-associated diseases. These pathogenic materials include cholesterol, oxidized cholesterol, and 7-ketocholesterol (implicated in heart disease and stroke), beta-amyloid plaques and neurofibrillary tangles in the brain (implicated in Alzheimer's disease), lipofuscin such as A2E in the retinal pigment epithelium (implicated in age-related macular degeneration), and extracellular matrix protein cross-links due to exposure of the tissue to high sugar levels such as carboxymethyllysine, carboxyethyllysine, Argpyrimidine, and other advanced glycation end products (implicated in diabetes).

Cells of the present disclosure can also be genetically modified to provide a therapeutic gene product that the patient requires, e.g., due to an inborn error of metabolism. Many genetic diseases are known to result from an inability of a patient's cells to produce a specific gene product. For example, a stem cell may be genetically modified to synthesize enhanced amounts of a gene product required by a patient. For example, hematopoietic stem cells that are genetically altered to produce and secrete adenosine deaminase can be prepared for transplant to a patient suffering from adenosine deaminase deficiency.

Preferably, the aforementioned genetic modifications are targeted modifications that avoid the risk of insertion at a site in the genomic DNA that disrupts normal cellular function, such as disruption of growth control that can cause neoplastic transformation. Alternatively, non-targeted methods may be used, such as using a recombinant retrovirus, and the insertion site(s) can then be identified to evaluate suitability of that cell for a particular use, for example by disqualifying cells where the insertion has the potential to disrupt a cell's normal growth control, and/or contains undesired viral sequences.

Methods of Identifying and Verifying Dedifferentiated Cells.

Candidate stem cells can be identified and verified using various methods. These methods include examining cell and colony morphology; determining whether the cells are immortal, for example by long-term growth in culture, measurement of telomere length, and/or measurement of telomerase activity; determining whether cells contain increased levels of pluripotency marker protein and/or mRNA, such as increased Alkaline Phosphatase, SSEA-1, Sox2, Oct4, Nanog, c-Myc, E-cad, Lin28, and Rex-1; decreased DNA methylation in the promoters of pluripotency genes such as Oct4 and Nanog; measurement of global gene expression; and detection of ability to differentiate in vitro and/or in vivo into cells in the three germ layers.

For example, in vivo differentiation can be determined by introducing cells into a developing embryo (such as by injection into a blastocyst, by aggregation with a blastocyte, and by other means known in the art) and detecting the presence of differentiated cells derived from the introduced cells. Differentiated cells that may be detected include neural progenitor cells (e.g., expressing Pax6), characteristic neurons (e.g., expressing TUJ1), mature cardiomyocytes (e.g., expressing CT3), definitive endoderm cells (e.g., expressing Sox17), pancreatic cells (e.g., expressing Pdx1), and hepatic cells (e.g., expressing ALB). In vivo differentiation can also be determined by injection of cells into immunodeficient mice, with developmental pluripotency being demonstrated by formation of teratoma-like masses similar to those that form upon injection of human ES cells (Adewumi, O. et al., Nature Biotechnol. 25, 803-816 (2007); Lensch et al., Cell Stem Cell 1, 253-258 (2007); Lensch et al., Nature Biotechnol. 25, 1211 (2007)).

Additionally, candidate stem cells can be analyzed to determine whether unwanted genetic and/or epigenetic alterations are present. For example, cells may be karyotyped, such as by cytological methods (including classic and spectral karyotyping methods) and/or by sequencing-based methods (e.g. digital karyotyping). Cells can also be tested to determine whether loss of heterozygosity has occurred, for example by comparing the genome-wide SNP profile between untreated cells and reprogrammed cells, with loss of heterozygosity indicating that potentially undesired recombination events have occurred (though in some instances loss of heterozygosity may be desired, for example to eliminate a particular unwanted allele). Additionally, cells can be tested to determine whether particular undesired sequences are present, e.g., undesired viral sequences, nucleic acids encoding reprogramming factors to which a cell has been exposed, mycoplasma and other pathogens, etc. Cells can also be tested to detect aberrant expression of oncogenes and/or tumor suppressors. Cells can also be tested for unwanted genome sequence modification by partial or full genome sequencing, which is optionally targeted to the sequences of particular genes (e.g. genes involved in growth regulation). Cells can also be tested for undesired epigenetic changes, such as undesired histone modification (Jenuwein et al., Science. 2001 Aug. 10; 293(5532):1074-80; Strahl et al., Nature. 2000 Jan. 6; 403(6765):41-5; Turner, Nat Cell Biol. 2007 January; 9(1):2-6).

Fusion Proteins

In certain exemplary embodiments of the present methods and compositions include fusion proteins. These fusion proteins contain domains or regions of proteins which are arranged differently than they are found in nature, for example by joining portions of different polypeptides.

Exemplary protein translocation domains (PTDs) include the HIV transactivating protein (TAT) (Tat 47-57) (Schwarze and Dowdy 2000 Trends Pharmacol. Sci. 21: 45-48; Krosl et al. 2003 Nature Medicine (9): 1428-1432). For the HIV TAT protein, an amino acid sequence sufficient to confer membrane translocation activity corresponds to residues 47-57 (YGRKKRRQRRR, SEQ ID NO: 1) (Ho et al., 2001, Cancer Research 61: 473-477; Vives et al., 1997, J. Biol. Chem. 272: 16010-16017). This sequence alone can confer protein transduction activity when attached to another polypeptide. The TAT PTD may also be the nine amino acids peptide sequence RKKRRQRRR (SEQ ID NO: 2) (Park et al. Mol Cells 2002 (30):202-8). The TAT PTD sequences may be any of the peptide sequences disclosed in Ho et al., 2001, Cancer Research 61: 473-477 (the disclosure of which is hereby incorporated by reference herein), including YARKARRQARR (SEQ ID NO: 3), YARAAARQARA (SEQ ID NO: 4), YARAARRAARR (SEQ ID NO: 5) and RARAARRAARA (SEQ ID NO: 6). Other proteins that contain PTDs include the herpes simplex virus 1 (HSV-1) DNA-binding protein VP22 and the Drosophila Antennapedia (Antp) homeotic transcription factor (Schwarze et al. 2000 Trends Cell Biol. (10): 290-295). For Antp, amino acids 43-58 (RQIKIWFQNRRMKWKK, SEQ ID NO: 7) represent are sufficient for protein transduction, and for HSV VP22 the PTD is represented by the residues DAATATRGRSAASRPTERPRAPARSASRPRRPVE (SEQ ID NO: 8). Alternatively, HeptaARG (RRRRRRR, SEQ ID NO: 9), or even larger poly-arginine peptides (e.g., having eight, nine, ten, eleven, etc. up to twenty or more arginine residues) or artificial peptides that confer transduction activity may be used as a PTD of the present disclosure.

In additional embodiments, the PTD may be a PTD peptide that is duplicated or multimerized. In certain embodiments, the PTD is one or more of the TAT PTD peptide YARAAARQARA (SEQ ID NO: 4). In certain embodiments, the PTD is a multimer consisting of three of the TAT PTD peptide YARAAARQARAYARAAARQARAYARAAARQARA (SEQ ID NO: 10). A protein that is fused or linked to a multimeric PTD, such as, for example, a triplicated synthetic protein transduction domain (tPTD), may exhibit reduced lability and increased stability in cells. Such a construct may also be stable in serum-free medium and in the presence of hES cells.

TABLE 3
Exemplary Protein Translocation Domains (PTDs)

| Protein Translocation Domain Sequence | SEQ ID NO: |
|---|---|
| YGRKKRRQRRR | 1 |
| RKKRRQRRR | 2 |
| YARKARRQARR | 3 |
| YARAAARQARA | 4 |
| YARAARRAARR | 5 |
| RARAARRAARA | 6 |
| RQIKIWFQNRRMKWKK | 7 |
| DAATATRGRSAASRPTERPRAPARSASRPRRPVE | 8 |
| RRRRRRR | 9 |
| YARAAARQARAYARAAARQARAYARAAARQARA | 10 |

Several proteins and small peptides have the ability to transduce or travel through biological membranes independent of classical receptor- or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. The small protein transduction domains (PTDs) from these proteins can be fused to other macromolecules, peptides or proteins to successfully transport them into a cell (Schwarze, S. R. et al. (2000) Trends Cell Biol. 10, 290-295). Sequence alignments of the transduction domains from these proteins show a high basic amino acid content (Lys and Arg) which may facilitate interaction of these regions with negatively charged lipids in the membrane. Secondary structure analyses show no consistent structure between all three domains. The advantages of using fusions of these transduction domains is that protein entry is rapid, concentration-dependent and appears to work with difficult cell types (Fenton, M. et al. (1998) J. Immunol. Methods 212, 41-48).

Alternatively or in addition to facilitate nuclear localization the reprogramming factor may be fused to one or more nuclear localization sequences. As mentioned already examples thereof include by way of example the SV40 T-antigen localization signal, the C-terminus of apoptin, acridine nuclear localization signal, polyargine (arg11), s4 13-PV, adenovirus hexon protein, PV-S4(13), RR-S4(13), et al. More generally, NLSs are often short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-.beta. nuclear localization signal (ARRRRP); NFκB p50 (EEVQRKRQKL; Ghosh et al., Cell 62:1019 (1990); NFκB p65 (EEKRKRTYE; Nolan et al, Cell 64:961 (1991); and others (see, for example, Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann. Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

Techniques for making fusion genes encoding fusion proteins are well known in the art. Essentially, the joining of various DNA fragments coding for different polypeptide sequences is performed in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

In certain embodiments, a fusion gene coding for a purification leader sequence, such as a poly-(His) sequence, may be linked to the N-terminus of the desired portion of a polypeptide or fusion protein, allowing the fusion protein be purified by affinity chromatography using a Ni++ metal resin. The purification leader sequence can then be subsequently removed by treatment with enterokinase to provide the purified polypeptide (e.g., see Hochuli et al., (1987) J. Chromatography 411:177; and Janknecht et al., PNAS USA 88:8972).

In certain embodiments, a protein or functional variant or active domain of it, is linked to the C-terminus or the N-terminus of a second protein or protein domain (e.g., a PTD) and/or NLS with or without an intervening linker sequence. The exact length and sequence of the linker and its orientation relative to the linked sequences may vary. The linker may comprise, for example, 2, 10, 20, 30, or more amino acids and may be selected based on desired properties such as solubility, length, steric separation, etc. In particular embodiments, the linker may comprise a functional sequence useful for the purification, detection, or modification, for example, of the fusion protein. In certain embodiments, the linker comprises a polypeptide of two or more glycines.

The protein domains and/or the linker by which the domains are fused may be modified to alter the effectiveness, stability and/or functional characteristics of the protein.

Transdifferentiation

Recent studies have shown the possibility of reprogramming an adult somatic or “terminally” differentiated cell to adopt a different cell fate. Turning a differentiated cell or the nucleus thereof into a differentiated cell or nucleus of another type allows creation of patient-specific cell types on demand by directly transforming patient cells of one type into another desired type. Embodiments of these methods include direct transdifferentiation of a somatic cell to another somatic cell, and dedifferentiation to a progenitor or an ES cell that could then be differentiated into the desired cell type.

Identification of Dedifferentiation Factors

Still another application of the present methods is for identification of the substance or substances found in cytoplasm that induces de-differentiation. This can be effected by fractionation of cytoplasm and screening these fractions to identify those which contain substances that result in effective rejuvenation or reprogramming when transferred into recipient cells, e.g., human differentiated cell types.

Under appropriate conditions, compounds present in the cytoplasm of donor cells provide for reprogramming or de-differentiation of recipient cells. These compounds likely include nucleic acids and/or proteinaceous compounds. Fractionation of donor cell cytoplasm allows enrichment (and ultimately identification) of those compounds. Fraction may include any of the numerous methods known in the art, including methods based on size, isoelectric point, charge, hydrophobicity, etc. Optionally, fractions suspected to contain a reprogramming agent may be treated to selectively ablate particular classes of agents (using nucleases, proteases, irradiation, etc.), thereby helping to determine the nature of the suspected reprogramming agent. Optionally, known reprogramming agents are depleted or inactivated in the cytoplasm or cytoplasmic fractions (e.g., by immunoaffinity depletion or addition of a neutralizing antibody) to facilitate detection of novel reprogramming agents.

For example, cells can be treated with a group of reprogramming factors that is known to be insufficient for robust reprogramming, and further treated with candidate reprogramming factor(s), with an increase in the rate of successful reprogramming indicating that a candidate factor could a reprogramming factor. Candidate reprogramming factors include proteins, nucleic acids, small molecules, siRNAs (including analogs), etc., which may be derived from a library, fractionated donor cell cytoplasm, selected due to homology to known reprogramming factors, selected due to known increased levels of expression in primitive cells or cells undergoing reprogramming, etc.

Cytoplasmic Transfer to De-Differentiate, Reprogram, or Rejuvenate Recipient Cells

Introduction

One aspect of the present disclosure provides novel methods for de-differentiating and/or altering the life-span of desired cells, preferably mammalian cells and, most preferably, human or other primate cells by the introduction of cytoplasm from a more primitive cell type, typically an undifferentiated or substantially undifferentiated cell, e.g., an oocyte or blastomere.

Nuclear transfer first gained acceptance in the 1960's with amphibian nuclear transplantation. (Diberardino, M. A. 1980, “Genetic stability and modulation of metazoan nuclei transplanted into eggs and ooctyes”, Differentiation, 17-17-30; Diberardino, M. A., N. J. Hoffner and L. D. Etkin, 1984; “Activation of dormant genes in specialized cells”, Science, 224:946-952; Prather, R. S. and Robl, J. M., 1991, “Cloning by nuclear transfer and splitting in laboratory and domestic animal embryos”, In: Animal Applications of Research in Mammalian Development, R. A. Pederson, A. McLaren and N. First (ed.), Cold Spring Harbor Laboratory Press.) Nuclear transfer was initially conducted in amphibians in part because of the relatively large size of the amphibian oocyte relative to that of mammals. The results of these experiments indicated to those skilled in the art that the degree of differentiation of the donor nucleus was greatly instrumental, if not determinative, as to whether a recipient oocyte containing such cell or nucleus could effectively reprogram said nucleus and produce a viable embryo. (Diberardino, M. A., N. J. Hoffner and L. D. Etkin, 1984, “Activation of dormant genes in specialized cells.”, Science, 224:946-952; Prather, R. S. and Robl, J. M., 1991, “Cloning by nuclear transfer and splitting in laboratory and domestic animal embryos”, In: Animal Applications of Research in Mammalian Development, R. A. Pederson, A. McLaren and N. First (ed.), Cold Spring Harbor Laboratory Press).

Much later, in the mid 1980s, after microsurgical techniques had been perfected, researchers investigated whether nuclear transfer could be extrapolated to mammals. The first procedures for cloning cattle were reported by Robl et al (Robl, J. M., R. Prather, F. Barnes, W. Eyestone, D. Northey, B. Gilligan and N. L. First, 1987, “Nuclear transplantation in bovine embryos”, J. Anim. Sci., 64:642-647). In fact, Dr. Robl's lab was the first to clone a rabbit by nuclear transfer using donor nuclei from earlier embryonic cells (Stice, S. L. and Robl, J. M., 1988, “Nuclear reprogramming in nuclear transplant rabbit embryos”, Biol. Reprod, 39:657-664). Also, using similar techniques, bovines (Prather, R. S., F L. Barnes, M L. Sims, Robl, J. M., W. H. Eyestone and N. L. First, 1987, “Nuclear transplantation in the bovine embryo: assessment of donor nuclei and recipient oocyte”, Biol. Reprod., 37:859-866) and sheep (Willadsen, S. M., 1986, “Nuclear transplantation in sheep embryos”, Nature, (Lond) 320:63-65), and putatively porcines (Prather, R. S., M. M. Sims and N. L. First, 1989, “Nuclear transplantation in pig embryos”, Biol. Reprod., 41:414), were cloned by the transplantation of the cell or nucleus of very early embryos into enucleated oocytes.

In the early 1990s, the possibility of producing nuclear transfer embryos with donor nuclei obtained from progressively more differentiated cells was investigated. The initial results of these experiments suggested that when an embryo progresses to the blastocyst stage (the embryonic stage where the first two distinct cell lineages appear) that the efficiency of nuclear transfer decreases dramatically (Collas, P. and J. M. Robl, 1991, “Relationship between nuclear remodeling and development in nuclear transplant rabbit embryos”, Biol. Reprod., 45:455-465). For example, it was found that trophectodermal cells (the cells that form the placenta) did not support development of the nuclear fusion to the blastocyst stage. (Collas, P. and J. M. Robl, 1991, “Relationship between nuclear remodeling and development in nuclear transplant rabbit embryos”, Biol. Reprod., 45:455-465). By contrast, inner cell mass cells (cells which form both somatic and germ line cells) were found to support a low rate of development to the blastocyst stage with some offspring obtained. (Collas P, Barnes F L, “Nuclear transplantation by microinjection of inner cell mass and granulosa cell nuclei”, Mol Reprod Devel., 1994, 38:264-267) Moreover, further work suggested that inner cell mass cells which were cultured for a short period of time could support the development to term. (Sims M, First N L, “Production of calves by transfer of nuclei from cultured inner cell mass cells”, Proc Natl Acad Sci, 1994, 91:6143-6147).

Based on these results, it was the overwhelming opinion of those skilled in the art at that time that observations made with amphibian nuclear transfer experiments would likely be observed in mammals. That is to say, it was widely regarded by researchers working in the area of cloning in the early 1990's that once a cell becomes committed to a particular somatic cell lineage that its nucleus irreversibly loses its ability to become “reprogrammed”, i.e., to support full term development when used as a nuclear donor for nuclear transfer. While the exact molecular explanation for the apparent inability of somatic cells to be effectively reprogrammed was unknown, it was hypothesized to be the result of changes in DNA methylation, histone acetylation and factors controlling transitions in chromatin structure that occur during cell differentiation. Moreover, it was believed that these cellular changes could not be reversed. Therefore, it was quite astounding that in 1998, the Roslin Institute reported that cells committed to somatic cell lineage could support embryo development when used as nuclear transfer donors. Equally astounding, and more commercially significant, the production of transgenic cattle which were produced by nuclear transfer using transgenic fibroblast donor cells was reported shortly thereafter by scientists working at the University of Massachusetts and Advanced Cell Technology.

Two calves were reportedly produced at the Ishikawa Prefecture Livestock Research Centre in Japan from oviduct cells collected from a cow at slaughter (Hadfield, P. and A. Coghlan, “Premature birth repeats the Dolly mixture”, New Scientist, Jul. 11, 1998). Further, Jean-Paul Renard from INRA in France reported the production of a calf using muscle cells from a fetus. (MacKenzie, D. and P. Cohen, 1998, “A French calf answers some of the questions about cloning”, New Scientist, March 21.) Also, David Wells from New Zealand reported the production of a calf using fibroblast donor cells obtained from an adult cow. (Wells, D. N., 1998, “Cloning symposium: Reprogramming Cell Fate—Transgenesis and Cloning,” Monash Medical Center, Melbourne, Australia, April 15-16)

Differentiated cells have also reportedly been successfully used as nuclear transfer donors to produce cloned mice. (Wakayama T, Perry A C F, Zucconi M, Johnsoal K R, Yanagimachi R., “Full-term development of mice from enucleated oocytes injected with cumulus cell nuclei”, Nature, 1998, 394:369-374.)

Still further, an experiment by researchers at the University of Massachusetts and Advanced Cell Technology was reported in a lead story in the New York Times, January 1999, wherein a nuclear transfer fusion embryo was produced by the insertion of an adult differentiated cell (cell obtained from the cheek of an adult human donor) into an enucleated bovine oocyte. Thus, it would appear, based on these results, that at least under some conditions differentiated cells can be reprogrammed or de-differentiated.

Related thereto, it was also recently reported in the popular press that cytoplasm transferred from oocyte of a young female donor “rejuvenated” an oocyte of an older woman, such that it was competent for reproduction.

However, it would be beneficial if methods could be developed for converting differentiated cells to embryonic cell types, without the need for cloning, and the production of embryos, especially given their potential for use in nuclear transfer and for producing different differentiated cell types for therapeutic use. Also, it would be beneficial if the cellular materials responsible for de-differentiation and reprogramming of differentiated cells could be identified and produced by recombinant methods, thereby improving the efficiency of cellular reprogramming.

Methods of Cytoplasmic Transfer to De-Differentiate, Reprogram, or Rejuvenate Recipient Cells

As noted above, it has been reported in the popular press that a group working in the area of artificial insemination and infertility successfully transferred the cytoplasm from the oocyte of a younger woman into that of an older woman and thereby rejuvenated the ability of the older oocyte to be competent for fertilization and embryo development. Based on this anecdotal evidence, coupled with recent papers in the scientific literature which suggest that differentiated adult cells may be effectively “reprogrammed” by nuclear transfer, it was theorized that differentiated cells could be effectively “reprogrammed” or “de-differentiated” and/or have their life-span altered (increased) by the introduction of cytoplasm from that of undifferentiated or substantially undifferentiated cell, e.g., an oocyte or blastomere or another embryonic cell type.

While it is presently unknown how the cytoplasm of one cell affects the life-span or state of differentiation of another, it is theorized that the cytoplasm of cells in early or primitive states of development contains one or more substances, e.g., transcription factors and/or other substances that act to trigger or promote cell de-differentiation. For example, one substance likely contained therein that affects the state of cell differentiation is telomerase. Another substance is OCT-4 and REX. However, Applicant does not wish to be bound to this theory as it is not necessary for an understanding of the disclosure.

In one aspect of the present disclosure, a recipient cell will typically be dedifferentiated in vitro by the introduction of an effective amount of cytoplasm from a donor cell, i.e., an undifferentiated or substantially undifferentiated cell, e.g., an oocyte or blastomere. This introduction or transfer of cytoplasm can be effected by different methods, e.g., by microinjection or by use of a liposomal delivery system. A preferred means comprises the introduction of cytoplasm blebs derived from ES cells, oocytes or other embryonic cells into desired differentiated cells, e.g. mammalian or other cells which are at or near senescence. For example, such cytoplasm blebs can be introduced into genetically modified mammalian cells in order to rejuvenate such cells, e.g. prior to their usage for cell therapy. Alternatively, cytoplasmic blebs can be contacted with nuclei from differentiated cells to induce rejuvenation.

The recipient cell can be of any species and may be heterologous to the donor cell, e.g., amphibian, mammalian, avian, with mammalian cells being preferred. Especially preferred recipient cells include human and other primate cells, e.g., chimpanzee, cynomolgus monkey, baboon, other Old World monkey cells, caprine, equine, porcine, ovine, and other ungulates, murine, canine, feline, and other mammalian species.

Also, the recipient cell can be any differentiated cell type. Suitable examples thereof include epithelial cells, endothelial cells, fibroblasts, keratinocytes, melanocytes and other skin cell types, muscle cells, bone cells, immune cells such as T and B-lymphocytes, oligodendrocytes, dendritic cells, erythrocytes and other blood cells; pancreatic cells, neural and nerve cell types, stomach, intestinal, esophageal, lung, liver, spleen, kidney, bladder, cardiac, thymus, corneal, and other ocular cell types, etc. In general, the methods have application in any application wherein a source of cells that are in a less differentiated state would be desirable.

As noted, the transferred cytoplasm will be obtained from a “donor” cell that is in a less differentiated state or more primitive state than the recipient cell. Typically, the cytoplasm will be derived from oocytes or cells of early stage embryos, e.g., blastomeres or inner cell mass cells derived from early stage embryos. In general, it is preferred that the donor cytoplasm be obtained from oocytes or other embryonic cells that are in an undifferentiated or substantially undifferentiated state. Bovine oocytes are a preferred source because they can be readily obtained in large quantities from slaughterhouses.

There have been reports in the literature concerning the production of cultures comprising embryonic stem cells that reportedly express or do not express certain markers characteristic of embryonic stem cells. It is therefore also preferable that donor cytoplasm be obtained from an oocyte or other cell that expresses or does not express cell markers which are characteristic of an undifferentiated, embryonic cell type. Such markers on primate ES cells include, by way of example, SSEA-1 (−); SSEA-3 (+); SSEA-4 (+); TRA-1-60 (+); TRA-1-81 (+); and alkaline phosphatase (+). (See U.S. Pat. No. 5,843,780 to Thomson, issued Dec. 1, 1998.)

As discussed above, it is also desirable that telomerase and/or a DNA sequence or other compound that provides for the expression of telomerase be introduced into the recipient cell, e.g., a mammalian cell and, more preferably, a human or non-human primate cell. The isolation of telomerase and cloning of the corresponding DNA has been reported previously. For example, WO 98/14593, published Apr. 9, 1998, by Cech et al, reports telomerase nucleic acid sequences derived from Eeuplotes aediculatus, Saccharomyces, Schizosaccharomyces, and human, as well as polypeptides comprising telomerase protein subunits. Also, WO 98/14592, to Cech et al, published Apr. 9, 1998, discloses compositions containing human telomerase reverse transcriptase, the catalytic protein subunit of human telomerase. Also, U.S. Pat. Nos. 5,837,857 and 5,583,414 describe nucleic acids encoding mammalian telomerases. Still further, U.S. Pat. No. 5,830,644, issued to West et al; U.S. Pat. No. 5,834,193, issued to Kzolowski et al, and U.S. Pat. No. 5,837,453, issued to Harley et al, describe assays for measuring telomerase length and telomerase activity and agents that affect telomerase activity. These patents and PCT applications are incorporated by reference in their entirety herein.

Thus, in one aspect of the present disclosure, desired cells, e.g., cultured human somatic cells, may be de-differentiated or reprogrammed in tissue culture by the introduction of cytoplasm of a more primitive cell type, e.g., an oocyte or embryonic cell type alone or in conjunction with telomerase. The introduction of cytoplasm from a donor oocyte or embryonic cell, e.g., blastomere, may be accomplished by various methods. For example, this can be effected by microsurgically removing part or all of the cytoplasm of a donor oocyte or blastomere or other embryonic cell type with a micropipette and microinjecting such cytoplasm into that of a recipient mammalian cell. It may also be desirable to remove cytoplasm from the recipient cell prior to such introduction. Such removal may be accomplished by well known microsurgical methods. Alternatively, the cytoplasm and/or telomerase or telomerase DNA can be introduced using a liposomal delivery system.

The present methods should provide a means of producing embryonic stem cells, e.g., mammalian embryonic stem cells, and most desirably, human embryonic stem cells, by reprogramming or de-differentiating desired cells in tissue culture. These cells are desirable from a therapeutic standpoint since such cells can be used to give rise to any differentiated cell type. The resultant differentiated cell types may be used in cell transplantation therapies.

Another significant application of the present disclosure is for gene therapy. To date, many different genes of significant therapeutic importance have been identified and cloned. Moreover, methods for stably introducing such DNAs into desired cells, e.g., mammalian cells and, more preferably, human somatic cell types, are well known. Also, methods for effecting site-specific insertion of desired DNAs via homologous recombination are well known in the art.

However, while suitable vectors and methods for introduction and detection of specific DNAs into desired somatic cells are known, a significant obstacle to the efficacy of such methods is the limited life-span of normal, i.e., non-immortal cells, in tissue culture. This is particularly problematic in situations wherein the introduction of multiple DNA modifications, e.g., deletions, substitutions, and/or additions is desired. Essentially, while methods for effecting targeted DNA modifications are known, the requisite time to effect and select for such modifications can be very lengthy. Thus, the cells may become senescent or die before the desired DNA modifications have been effected. The present disclosure provides methods that can alleviate this inherent constraint of gene and cell therapy by introducing the cytoplasm of an oocyte or other embryonic cell type into recipient cells prior, concurrent or subsequent to genetic modification. The introduction of such cytoplasm alone or in combination with telomerase or a DNA or another compound that results in the expression of telomerase, will reprogram the genetically modified cell and enable it to have a longer life-span in tissue culture. Such reprogramming can be effected once or repeatedly during genetic modification of recipient cells. For example, in the case of very complex genetic modifications, it may be necessary to “reprogram” recipient cells several times by the repeated introduction of donor cytoplasm to prevent senescence. The optimal frequency of such reprogramming will be determined by monitoring the doubling time of the cells in tissue culture such that the cells are reprogrammed before they become senescent.

The resultant reprogrammed genetically modified cells, which have a longer life-span as a result of reprogramming, may be used for cell and gene therapy. Moreover, these cells may be used as donor cells for nuclear transfer procedures or for the production of chimeric animals. The present methods will make it possible to produce cloned and chimeric animals having complex genetic modifications. This will be especially advantageous for the production of animal models for human diseases. Also, the present methods will be beneficial in situations wherein the expression of a desired gene product or phenotype is dependent upon the expression of different DNA sequences, or for gene research involving the interrelated effects of different genes on one another. Moreover, it is anticipated that the present methods will become very important as the interrelated effects of the expression of different genes on others becomes more understood.

Yet another application of the present disclosure is for alleviating the effects of aging. Just as mammalian cells have a finite life-span in tissue culture, they similarly have a finite life-span in vivo. This finite life-span is hypothesized to explain why organisms, including humans, have a normal maximum life-span, determined by the finite life-span of human somatic cells.

The present application provides methods to alleviate the effects of aging by taking mammalian cells from an individual and altering (lengthening) the life-span of such cells by introduction of cytoplasm from an oocyte or other embryonic cell type, e.g., blastomere. The resultant rejuvenated cells may be used to produce differentiated cell types in tissue culture and these cells can then be introduced into the individual. This can be used, e.g., to rejuvenate the immune system of an individual. Such rejuvenation should be useful in the treatment of diseases thought to be of immune origin, e.g., some cancers.

Also, the subject methods may be used for the production of autologous grafts, e.g., skin grafts, which can be used in the case of tissue injury or elective surgery.

Yet another application of the present disclosure is for treating the effects of chronologic and UV-induced aging on the skin. As skin ages, various physical changes may be manifested including discoloration, loss of elasticity, loss of radiance, and the appearance of fine lines and wrinkles. It is anticipated that such effects of aging may be alleviated or even reversed by topical application of cytoplasm-containing compositions. For example, cytoplasm from donor oocytes, e.g., bovine oocytes, optionally further including telomerase or a telomerase DNA construct, can be packaged in liposomes to facilitate internalization into skin cells upon topical application. Also, it may be advantageous to include in such compositions compounds that facilitate absorption into the skin, e.g., DMSO. These compositions may be topically applied to areas of the skin wherein the effects of aging are most pronounced, e.g., the skin around the eyes, the neck and the hands.

Still another application of the present disclosure is for identification of the substance or substances found in cytoplasm that induces de-differentiation. This can be effected by fractionation of cytoplasm and screening these fractions to identify those which contain substances that result in effective rejuvenation or reprogramming when transferred into recipient cells, e.g., human differentiated cell types.

Alternatively, the component(s) contained in oocyte cytoplasm responsible for reprogramming or rejuvenation can be identified by subtractive hybridization by comparing mRNA expression in early stage embryos and oocytes to that of more differentiated embryos.

Notwithstanding the identification of factors sufficient to effect dedifferentiation of somatic cells in some experimental systems, the component or compounds contained in embryonic cell cytoplasm are responsible for cell reprogramming or de-differentiation may not have been fully identified. In fact, it is uncertain even as to the specific nature of all such component(s), e.g., whether they are nucleic acids or proteins.

Such component(s) may comprise nucleic acids, in particular maternal RNAs, or proteins encoded thereby. In this regard, it has been reported by different groups that very early stage embryos contain a class of RNA known as maternal RNA's that are stored in the egg very early on but which are not detected past the blastula stage. (Kontrogianni-Konstantopoulos et al, Devel. Biol., 177(2):371-382 (1996).) Maternal RNA levels have been quantified for different species, i.e., rabbit, cow, pig, sheep and mouse. (Olszanska et al, J. Exp. Zool., 265(3):317-320 (1993).) With respect thereto, it has also been reported that maternal RNA in Drosophila oocyte encodes a protein that may bind to a tyrosine kinase receptor present in adjacent follicle cells that may initiate various events leading to dorsal follicle cell differentiation which act to delimit and orient the future dorsoventral axis of the embryo. (Schupbach et al, Curr. Opin. Genet. Dev., 4(4):502-507 (1994).)

Also, fractionation of oocytes has shown that mitogen-activated protein kinases are expressed at higher levels in small oocytes, suggesting that it is a maternal RNA that is stored for early embryogenesis. This is speculated to be involved in signal transduction in embryonic as well as adult cells. (Zaitsevskaya et al, Cell Growth Differ., 3(11):773-782 (1992).)

Still further, it has been reported that a maternal mRNA in silkworm oocytes encodes a protein that may be a structural component necessary for formation of the cellular blastoderm of the embryo, and that the association of such maternal mRNA with cortical cytoskeleton may participate in the synthesis of new cytoskeleton or related structures during blastoderm development. (Kastern et al, Devel., 108(3):497-505 (1990).)

Moreover, it has been reported that maternal poly(A)+RNA molecules found in the egg of the sea urchin and amphibian oocyte are completed with U1 RNA, a co-factor in somatic nuclear pre-mRNA splicing and that such RNAs contain repeated sequences interspersed with single-copy elements. (Calzone et al, Genes Devel., 2(3):305-318 (1988); Ruzdijic et al, Development, 101(1):107-116 (1987).)

Thus, based thereon, and the observation that cytoplasm apparently contains some component that results in cell reprogramming, it should be possible to identify compounds, likely nucleic acids and/or proteinaceous compounds which are present in the cytoplasm of oocytes and early embryos that, under appropriate conditions, provide for reprogramming or de-differentiation of desired cells. This will be effected by fractionation of cytoplasm into different fractions, e.g., based on size or isoelectric point, and ascertaining those factors which effect de-differentiation or reprogramming when transferred to differentiated cell types.

Alternatively, the factors responsible for reprogramming may be identified by subtractive or differential hybridization, essentially by identifying those mRNAs which are present in oocytes that are lost after the embryo has differentiated beyond a certain stage, e.g., past the blastula stage of development, and identifying those of which are involved in de-differentiation or reprogramming.

Therefore, the disclosure includes methods for the identification of the specific cytoplasmic materials, e.g., polypeptides and/or nucleic acid sequences, which when transferred into a differentiated cell provide for de-differentiation or reprogramming. Based on what has been reported with respect to maternal RNAs, it is anticipated that the active materials responsible for de-differentiation or reprogramming may include maternal RNAs or polypeptides encoded thereby.

After such nucleic acid(s) or polypeptides have been identified and sequenced, they will be produced by recombinant methods. It is anticipated that these recombinantly produced nucleic acids or polypeptides will be sufficient to induce reprogramming or de-differentiation of desired cells.

The present disclosure further encompasses assays wherein oocyte cytoplasm or cytoplasm from ES cells is fractionated into different fractions, e.g. based on molecular weight, isoelectric point, gel filtration, and salt precipitation, which are added into different microwells that contain one or more isolated nuclei from desired differentiated cells, e.g., mammalian, amphibian, avian, or insect cells and a screening assay conducted to identify mRNAs such as REX or OCT-4 that are released from the nuclei. For example, such mRNAs may be identified by PCR amplification and detection.

Alternatively, PCR screening assays may be conducted wherein ooplasm can be added to desired differentiated cells and assays conducted to identify what mRNAs, e.g. REX or OCT-4, are released from the cell nuclei after introduction of the oocyte cytoplasm.

The identification of such mRNAs can be identified by known methods, e.g. subtractive hybridization, differential display, and differential hybridization techniques. Essentially, these methods provide for the comparison of different populations of mRNAs in different cells, or cells at different times, and are conventionally used to identify genes that are expressed only under specific conditions or by specific types of cells.

In particular, subtractive hybridization can be effected by use of oocyte RNAs which are subtracted with RNAs obtained from normal somatic cell RNAs. Thereby, RNAs that are involved in cell reprogramming can be identified.

Additionally, the present disclosure further includes the reconstitution of nuclei isolated from desired differentiated cells, e.g. those which are derived from differentiated cells in tissue culture, which potentially may be genetically modified by contacting such isolated nuclei with cytoplasm fractionated from oocytes, blastomeres or ES cells, and the addition of such reconstituted nuclei to cytoplasts, thereby producing a rejuvenated cell having increased proliferation potential and lifespan.

Trans-Differentiation and Re-Differentiation of Somatic Cells and Production of Cells for Cell Therapies

Introduction

Stem cells obtained from adults (mesenchymal, hematopoietic, neuronal) are receiving increasing interest as a source of material for cell and tissue transplantation to treat human disease. To a large degree, this interest has been stimulated by findings that report the presence of certain types of stem cells in unexpected tissue compartments in vivo (e.g. neuronal stem cells in bone marrow). In addition, some types of stem cells are displaying an unanticipated plasticity in their ability to trans-differentiate into other types of cells when transplanted from their niche into heterologous tissue compartments. Despite these developments, problems of stem cell accessibility and quantity persist.

The transdifferentiation potential of adult cells has also been receiving increasing attention (Eguchi and Kodama, 1993). Trans-differentiation is a physiological process that occurs during development but has also been described in a number of adult organs including liver, thyroid, mammary gland (Hay and Zuk, 1999), and kidney (Strutz et al., 1995). It has been shown that alteration of cell morphology and function can be induced artificially in vitro by treatment of cell cultures with cytoskeletal disruptors, hormones, and Calcium-ionophores. Trans-differentiation is a physiological process that occurs during development, and has also been described in a number of adult organs including liver, thyroid, mammary gland (Hay and Zuk, 1999), and kidney (Ng et al., 1999). Alteration of cell fate can be induced artificially in vitro and there is a vast amount of published data describing trans-differentiation. For example, embryonic blastomeres can be induced to differentiate in the presence of microfilament inhibitors (Okado and Takahashi, 1988, 1990; Wu et al., 1990; Pratt et al., 1981). Supplementing growth media for somatic cells with cytoskeletal inhibitors (Brown and Benya, 1988; Takigawa et al., 1984; Shea, 1990; Tamai et al., 1999; Cohen et al., 1999; Fernandez-Valle et al., 1997; Yujiri et al., 1999; Ulloa and Avila, 1996; Ferreira et al., 1993; Sato et al., 1991; Zanetti and Solursh, 1984; Kishkina et al., 1983; Hamano and Asofsky, 1984; Holtzer et al., 1975; Cohen et al., 1999), Ca-ionophores (Shea, 1990; Sato et al., 1991), corticosteroids (Yeomans et al., 1976), and DMSO (Hallows and Frank, 1992), causes changes in cell shape and function. Mammary epithelial cells can be induced to acquire muscle-like shape and function (Paterson and Rudland, 1985), spleen cells can be induced to produce both IgM and IgG immunoglobulins (van der Loo et al., 1979), pancreatic exocrine duct cells can acquire insulin-secreting, endocrine, phenotype (Bouwens, 1998a,b), 3T3 cells into adipose cells (Pairault and Lasnier, 1987), mesenchymal cells into chondroblasts (Rosen et al., 1986), bone marrow cells into liver cells (Theise et al., 2000), islets into ductal cells (Yuan et al., 1996), muscle into 7 non-muscle cell types, including digestive, secretory, gland, nerve cells (Schmid and Alder, 1984), muscle into cartilage (Nathanson, 1986), neural cells into muscle (Wright, 1984), bone marrow into neuronal cells (Black, 2000).

Methods of Trans-Differentiation and Re-Differentiation of Somatic Cells and Production of Cells for Cell Therapies

This section describes compositions and methods for trans-differentiation of cells in vitro that can avoid use of early preimplantation embryos, fetal tissues, or adult stem cells and can be customized for individual patients using their own cells as donors.

These methods utilizes a cell's ability to respond to environmental factors after they have been “primed” to do so in vitro. Priming in this context is achieved by destabilizing cell's cytoskeletal structure, consequently removing the feedback mechanisms between cell's shape and nuclear function. Both, shape and function define the specificity of any cell type. The human cell types used as a source are differentiated somatic cells, such as fibroblasts and keratinocytes from skin biopsies, and leukocytes from blood samples. Cell structure is first destabilized with cytoskeletal inhibitors, consequently their nuclear structure becomes permissive to alteration and upon exposure to conditions that promote or support a desired cell type such that the primed cells acquire this new morphology and function. Primed cells are multipotent and, upon application of factors that induce formation of the central nervous system are capable of differentiating into different neurons, astrocytes, or oligodendrocytes. The result is populations of newly differentiated neuronal cell types genetically identical to the fibroblasts sampled from the donor. These methods overcome barriers and limitations to the derivation of patient-specific cells, which are: the need for embryos as a source of embryonic stem cells, histo-incompatibility between the donor and the recipient, the risk of transmitting animal viruses via xenotransplantation, insufficient quantities of cells/tissues for transplantation, and high cost associated with generation of embryos and embryonic stem cells, life-long immunosuppression, and the requirement for repeated treatments.

These methods can be used to effect trans-differentiation of any type of any type of somatic cell into any other type of somatic cell. Examples of such cells that may be used or produced include fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, macrophages, monocytes, and mononuclear cells.

The cells used with these methods may be of any animal species; e.g., mammals, avians, reptiles, fish, and amphibians. Examples of mammalian cells that can be transdifferentiated by these methods include but are not limited to human and non-human primate cells, ungulate cells, rodent cells, and lagomorph cells. Primate cells with which these methods may be performed include but are not limited to cells of humans, chimpanzees, baboons, cynomolgus monkeys, and any other New or Old World monkeys. Ungulate cells with which these methods may be performed include but are not limited to cells of bovines, porcines, ovines, caprines, equines, buffalo and bison. Rodent cells with which these methods may be performed include but are not limited to mouse, rat, guinea pig, hamster and gerbil cells. Rabbit cells are an example of cells of a lagomorph species with which these methods may be performed.

Using the present methods, cells of one differentiated cell type can be converted to a different differentiated cell type without necessarily reverting to a stem-like cell intermediate. This may be done without losing cell viability, and while and allows the converted cells to retain their overall biochemical activity and chromatin stability.

An exemplary embodiment of the present method comprises sequentially evaluating each of the steps required for trans-differentiation. The steps include: 1. growth of primary cell cultures, effectiveness and reliability of “priming” agents, assessment of the primed state in vitro, 2. the ability of primed cells to trans-differentiate upon induction, 3. design reproducible and reliable induction protocols, 4. ability to maintain stable cell function, and 5. the ability of newly trans-differentiated cell types to interact with patient's cells upon cell transplantation. Primed and newly induced cell types can be characterized for their gene expression, cell surface antigens, morphology, excitability, secretory function, synapse formation, and stable functional grafting in the rat model for Parkinson's disease.

Pharmaceutical strategies for treating a variety of neuronal disorders are currently available, but all of these organic chemicals have limitations in their clinical efficacy. For example, the most widely used drug for treatment of Parkinson's disease, Levodopa is a dopamine precursor and results in increased dopamine production from dopaminergic neurons. However, side effects of Levodopa are debilitating and include hallucination, severe nausea, and vomiting. Long-term use results in induction of tolerance, which in turn translates to increasing doses over time, ultimately leading to a lower clinical benefit to risk ratio. Treatment of brain disorders using biologics is not practical since the therapeutic agent must cross the blood-brain barrier, which does not happen for most proteins and peptides present in the bloodstream. Due to the limitations of traditional pharmaceutical and biologic intervention, alternative approaches are being pursued aggressively. Recent advances in in vitro cell culture and manipulation technology have led to the prospect of using cell transplantation as a means for restoring cells or tissues that have been damaged due to progression of disease. This approach not only offers the prospect of treating the disease, but also may ultimately provide a cure, if the grafted cells become fully integrated and functional upon transplantation into the host tissue. Currently, there are three major target areas for obtaining material for cell transplantation therapy for treatment of candidate diseases such as Parkinson's disease and other neurological disorders. Each of these approaches is discussed in turn.

First, recent derivation of both monkey and human embryonic stem-like cells from blastocyst inner cell masses has enabled investigation of differentiation events that has not been possible in primates before. Embryonic stem-like cells have been shown to develop into lineages of all three germ layers in vitro. Consequently, many research groups are focusing their resources on the use of therapeutic cloning approaches, which uses mammalian oocytes as a vehicle to exploit factors important for genomic reprogramming. However, the potential use of specialized cells derived from ES-like cells for allo-transplantation in humans has yet to be evaluated (Bain et al., 1995; Brustle et al., 1999; Fairchild et al., 1995; Keller, 1995).

Second, an alternative approach to provide patients with highly specialized cell types relies on a non-embryonic stem cell as an intermediate. Populations of tissue-specific progenitor cells, such as mesenchymal, hematopoietic, and neuronal stem cells are obtained from specific locations within an adult human patient. These adult tissue-specific stem cells have been isolated, propagated in vitro, and astonishing progress has been achieved in differentiation of mesenchymal and neuronal precursors into adipocytic, chondrocytic, osteocytic cells, blood cells, and neurons, respectively (Pittenger et al., 1999; Black et al., 2000). Using this approach, histo-incompatibility between donor cells and recipient is alleviated. A major disadvantage is that the process requires cumbersome clinical and laboratory procedures that are not fully established to obtain sufficient quantities of progenitor stem cells from adults.

Finally, a third strategy involves xeno-transplantation using pig cells as donors. The most advanced program involves obtaining neurons from pig fetuses and transplanting them into human patients with minimal in vitro manipulation. On average, 8 fetuses are required for treatment of a single patient, which limits usefulness of this approach (Studer, personal communication). In addition, recent concern over transmission of porcine viruses to humans has slowed otherwise effective and promising research in this area (Imaizium et al., 2000).

Despite these discoveries, the path to development of various tissue specific cell types without embryonic or other stem cells as an intermediate requirement has not been described. Consequently, there are strong justifications across the entire spectrum of biomedical research for developing alternative methods for production of patient-compatible or autologous specific cell types. A reliable source of cells would be needed to treat millions of patients affected with Parkinson's disease, Huntington's disease, Alzheimer's disease, Multiple Sclerosis, spinal cord injuries, stroke, burns, heart disease, diabetes, arthritis, and many genetic and other disorders that could benefit from cell/tissue therapy. The ability to use embryonic and/or adult stem cells in reliable and efficient strategies for the production of specialized cell types and tissues for human cell/tissue therapy remains to be shown. Notwithstanding these successes and exciting prospects, the problem of histo-incompatibility between the donor and the recipient of stem cells remains unsolved, as does the availability of preimplantation embryos for the derivation of embryonic stem cells.

Various types of differentiated neuronal cells can be generated from a single type of somatic cell taken from an individual donor (primary cell cultures) and the resulting cells transplanted into the same individual. The present methods provide for effecting trans-differentiation of highly specialized somatic cells (e.g. skin fibroblasts) into different, fully functional specialized cells (dopaminergic neurons, astrocytes, oligodendrocytes, GABA neurons, serotonin neurons, acetylcholin transferase neurons, etc.) in vitro. The present method does not require utilizing any part of an oocyte, an early preimplantation embryo or fetal tissue as a vehicle for de-differentiation and reprogramming. It can be customized for individual patients.

The present method exploits the fact that all the cells of an individual contain all the genetic information required for development. Expression of specific genes that define a cell's morphology and function is determined largely by genetic programming and environmental signals, but can be altered upon environmental insults (as in wound healing, bone regeneration, and cancer). In order to change the function of the cell, the present method uses cytoskeletal disruptors for “priming” of differentiated cells. Our hypothesis is that priming alters the cytoskeleton, which disrupts the cell's transport machinery, and ultimately interferes with the cell type-specific feedback mechanisms the nucleus receives from the cell's periphery. This disruption allows the nucleus to become responsive to different or alternative clues from its environment. After priming, cells are exposed to an environment that induces and supports differentiation into the desired cell type (i.e. neurobasal medium for neurons).

The benefits of the present method are significant and include:

(i) No need for embryos or fetal tissue. With the present method, human embryos do not have to be created, destroyed, or used to generate trans-differentiated cells, thus eliminating the cost of production, time constraints, and the concern over ethical issues.

(ii) No need for patient immuno-suppression. Efficacious allo- and xeno-cell therapy protocols have been demonstrated in many pre-clinical animal models and in some clinical human subjects. However, in most cases, extended graft survival (beyond a few days) has only occurred when combined with pharmaceutical immuno-suppression. This includes cases where the cells are encapsulated with artificial matrix materials, such as alginate, which is designed to exclude histocompatibility molecules. While the matrix encapsulation approach may reduce short-term graft rejection, eventually the transplanted cells are destroyed due to nutrient and oxygen deprivation, which results from pericapsular fibrosis. This results in the need for repeated treatment. Therefore, a preferred method of long term and lasting treatment using cell-based therapy would involve cells originally derived from the patient.

(iii) No health risks due to possible transmission of animal viruses. The present method avoids the concerns in xenotransplantation regarding porcine endogenous retroviruses (PERVS). PERVS are ancestral genes located in the porcine genome that resulted from retroviral DNA integration. There is a possibility of that the presence of porcine cells in the human body could induce PERV expression in an immuno-suppressed patient that might lead to recombination, thus, producing new pathogens. This would pose a new health threat not only to the patient, but also to the surrounding population if the new virus were to be communicable. Since no component of an animal cell is ever used in the method, threats due to animal genomic DNA sequences such as PERVS are not a concern.

(iv) Availability of large numbers of specialized cells in a relatively short time. The present method contrasts with embryonic methods, which have yielded only small numbers of starting stem cells (between 10-15 cells from a blastocyst). The current strategies being developed by our competitors (Geron, Menlo Park, Calif.) utilize established human embryonic stem cell lines as the basis for their product. Since the number of cells used to derive the initial cell line is so low, a vast amount of in vitro proliferation will have to take place to satisfy the needs of the millions of patients to be treated with cell therapy. It is known that extensive proliferation in vitro results in acquired genetic mutations and even spontaneous imortalization. Since in the present method large numbers of cells are be harvested from individual patients (a single, common source of stem cells is not required any longer) as starting material, the degree of in vitro proliferation is only what is needed to prime the cells, trans-differentiate them and generate enough cells for a needed clinical application.

(v) Lower cost. The present method will significantly reduce the cost of cell therapy by eliminating the need for immuno-suppression of the patient to reduce hyperacute (in xeno-transplantation) and delayed rejection (in allo- and xeno-transplantation). Using current transplantation regimes, patients depend on lifetime immuno-suppressive therapy, which is not only costly, but results in increased risk of infections, and lower quality of life. The need for repeated transplantation procedures would likely also be alleviated.

Donor cells are treated in a way that “primes” them for trans-differentiation without reverting them necessarily to stem-like cells. This is done without losing cell viability and allowing them to retain their overall biochemical activity and chromatin stability; in short, to ensure the cells can retain their overall functionality.

In vivo, differentiated cell types vary in their ability to undergo proliferation and continue cycling upon physiological demand. Several cell types are known to be terminally arrested in the G0 phase of the cell cycle and do not proliferate after birth. Examples are heart smooth muscle cells, neurons, Sertoli cells in male testes, and oocytes in female ovaries. Other cell types, however, have been known to have high regeneration ability that is retained during the post-natal period. They include liver cells, several connective tissue cell types (cartilage, bone, and fibroblasts), epithelia (skin and gut); hematopoietic cells (bone marrow and spleen) and this regeneration response can generally be induced by trauma. Not only can these cells regenerate themselves but can also generate cells of distinctly different phenotypes. Transdifferentiation potential of adult cells has been receiving increasing attention (Eguchi and Kodama, 1993; Strutz and Muller, 2000).

This method describes technology for trans-differentiation of one type of somatic cell into another using in vitro culture with cytoskeletal inhibitors (cytochalasins A, B, D and E, latrunculin, jasplakinolide, etc). It further describes technology for maintenance of the newly trans-differentiated cell types, stable cell morphology and cell-specific gene and protein expression. The utility of the present method is in developing specific growth factor, matrix and cytokine combinations that reliably direct differentiation into a desired cell type. This provides autologous (isogeneic) cell types for cell transplantation in the same individual that donated the initial somatic cell sample. The present method overcomes immune rejection by the cell transplantation recipient, significantly shortens the time required for the “new” cells to be available for therapy, does not use embryo or fetus intermediaries as vehicles for reprogramming, and does not require generation of embryonic or any other stem cell precursors. The present method produces cells that are primed to develop into neuronal cell lineages. During the period of time when the cells are being “primed” they may be used as partially de-differentiated cells for derivation of other, non-neuronal cell types.

1. Develop an Interaction Matrix Between Donor Cell Type, Optimal Cell Cycle Stage and the Priming Agent.

Rationale: Terminally differentiated somatic cells of the vast majority of mammalian tissues lose their genomic plasticity during development. Cells are characterized to belong to a specific tissue based on their location in vivo, their morphological appearance (shape and size), expression of specific proteins, and specialized function. Some of these characteristics are retained when cells are isolated from an individual and propagated in culture. Conditions that support extensive expansion of various cell types in culture are well established, as are requirements for maintenance of their morphology and function largely due to the fact that maintenance of the desired cell type was the experimental goal (Basic Cell Culture Protocols, 1997). It has also been recognized that among factors that determine a cell's fate during development and differentiation, the environment and clues received from neighboring cells and extra-cellular matrix (Hohn and Denker, 1994) not only promote proliferation of certain cell types but also determine their terminally differentiated phenotype (Fuchs et al., 2000). Several cell types can be induced to trans-differentiate in culture, such as bone marrow into brain cells (Black et al., 2000) and liver (Theise et al., 2000), muscle into chondrocytes (Nathanson, 1986), thyroid cells into neurons (Clark et al., 1995), and mammary epithelium into muscle (Paterson and Rudland, 1985). Though possible, trans-differentiation of fibroblasts has not been examined, and a “primed” state that allows trans-differentiation to occur has not been described for any cell type.

Experimental: Factorial experiments can be designed to investigate the interactions between donor cell type, primer, the concentration of the primer, duration of priming and the cell cycle stage of the donor cell. Human primary keratinocytes, fibroblasts, leukocytes, and liver cells obtained from commercially available sources (Clonetics, Calif. and ATCC, Rockville, Md.), will be grown in vitro and expanded to 1×10̂7 using standard cell culture conditions (DMEM, supplemented with amino acids, L-glutamine, .beta.-mercaptoethanol, 10% fetal calf serum; Gibco, Gaitherburg, Md.). Cultures will be supplemented with and increasing dose of cytochalasin B (CB, 0.1-10 μg/ml; Sigma Chemical Co, St. Louis, Mo.) and cells' morphology recorded at 12-hour intervals over a period of 72 hours. Control cells can be grown in vitro in the absence of the inhibitor or in the presence of DMSO (Sigma) alone, which is used to solubilize CB. At the same experimental time points, cells will be examined for down-regulation/loss of their specific gene/protein expression by RT-PCR and immuno-cytochemistry (ICC) according to published protocols. The majority of the oligonucleotide primers and antibodies for these studies are commercially available. Some of the markers are summarized in Table 4. In parallel, the effect of other microfilament inhibitors (cytochalasin A, D and E) will be examined at concentrations and times described above.

Cells are synchronized in G1, S, G2, and M-phase of the cell cycle using published protocols (Leno et al., 1992). Briefly, growing primary cultures are synchronized by an initial S phase block for 20 hours with 2.5 mM thymidine, followed after a 5 hour interval by a 9 hour mitotic block by demecolcine. Mitotic cells are shaken off and mitotic index checked on cytospin prepared slides. Double thymidine block (thymidine for 17 hours, release for 9 hours, thymidine for 15 hours) are used for synchronization of cells at the beginning of S phase. Seven hours after release of the second thymidine block, cells are expected to accumulate in G2. Synchronized cell populations are then exposed to CB as described for non-synchronized, randomly cycling cell populations.

TABLE 4
Donor cell types and associated endogenous, phenotype-specific markers.

| Donor cell type | Endogenous markers |
|---|---|
| Skin fibroblasts (mesoderm) | FSP-1, vimentin, fibronectin |
| Keratinocytes (ectoderm) | keratin, melanin |
| Hepatocytes (endoderm) | fibrinogen, albumin, cytokeratins 8, 18, 19 |
| Blood cells (mesoderm) | immunoglobulins, CD antigens |

Data Collection and Analysis: Changes in cell shape and general morphology are used as the first indicator of “priming” and images sequentially recorded by time lapse video imaging (cooled CCD camera, 40× magnification, DIC optics on an upright Olympus, Metamorph imaging software). Patterns of the down-regulation/loss of primary cell-specific gene expression and consequently protein synthesis are evaluated by RT-PCR and ICC and compared to control primary cells that were grown in culture for the same period of time, but were not exposed to the inhibitor, or were exposed to the same concentration of DMSO, which is used as a solvent for cytochalasin B.

Adherent cells, such as fibroblasts will change morphology due to cytoskeletal inhibition. Cells grown in suspension (blood cells) may display less or no morphological alteration. It is likely that cells will continue with nuclear progression through the cell cycle and karyokinesis, while cytokinesis will be inhibited. Depending on the dose of CB, cells may complete one or more rounds of DNA replication and karyokinesis in the absence of cell division. Cells “primed” with cytochalasin B lose their cell-specific gene expression, and this down-regulation is expected to correlate with the concentration of the priming agent and the duration of exposure. There are advantages and disadvantages of both low and high CB concentration priming protocols. Lower concentrations of CB may induce slow, step-by-step disruption of cellular architecture. This would in turn allow cells to gradually decrease cellular transport of tissue-specific factors to their cytoplasmic or plasma membrane targets. If inhibition is then maintained over an extended period of time, function of the cell's nucleus will gradually become deprived of feedback signals originating from target sites.

Without this feedback regulation, the nucleus would adopt a different gene expression profile which becomes less dependent on clues received from the immediate environment. A potential disadvantage of the low dose protocol is that viability of cells may decline, as the incubation time has to be lengthened. The environment can be manipulated in such a way that factors which are beneficial for neuronal development (ascorbic acid, all-trans retinoic acid, neuro-basal growth medium, bFGF and fibronectin) will allow for gradual, instead of abrupt, imposition of the cell to change. On the other hand, high concentration of CB for shorter period of time may be advantageous when high amounts of specific protein are required for a short period of time to maintain cell function (such as hormone secreting, endocrine cells). During exposure to high concentrations of CB, cells continue to replicate their DNA and progress through mitosis (karyokinesis) in the absence of cytokinesis only once. Multiple nuclei within primed cells are expected after low dose protocol and effect of multiple nuclei on cell function will have to be evaluated. Under high concentrations of CB bi-nucleated cells are expected to be the predominant outcome. In addition to cell type, the stage of the cell cycle during which a particular cell type is exposed to “primers” will result in different outcomes after priming. Our preliminary results show that cells can be kept viable for at least 72 hours in the presence of CB without detrimental effects on their survival.

Experiments can be performed using the methodology disclosed herein to test various values of parameters influencing the trans-differentiation process to develop a database of interactions. Such a database will permit one to predict the results of using a specific cell type, a specific primer, specific concentration and time of exposure, in terms of obtaining a cell of a desired morphology and gene down-regulation pattern.

It has been shown that DMSO can induce change of function on its own (Hallows and Frank, 1992). If control experiments indicate that this is indeed a possibility, we will examine effect of DMSO alone in more detail and design experiments accordingly. Using the disclosed methods, we have found that fibroblasts respond to CB treatment with a high degree of repeatability and that virtually all the cells display a change in phenotype, making them a cell type of choice for trans-differentiation. However, alternative cell types such as keratinocytes or white blood cells can also be used. Source cells selected for use should be easy to obtain, with minimal invasion and discomfort for the patient. If no distinct differences can be found between different donor cell types, fibroblasts can be used.

Different cytoskeletal inhibitors will induce distinctly different alteration in cells during priming. Cytoskeletal inhibitors that are suitable for use in the present method include microfilament disruptors (e.g., cytochalasin B, D, A, E; vimentin, latrunculin, jasplakinolide). These inhibitors act through different cellular targets in order to depolymerize microfilament network and a specific mode of action may be advantageous/disadvantageous for “priming” purposes. Different priming agents are expected to induce different “primed” state: for example, CB may be “priming” cell for neuronal development while cytochalasin D may be “priming” the same cells to undergo hematopoietic development (confidential preliminary data, not disclosed).

Microtubule inhibitors, such as colchicine, colcemid, nocodazole, and taxol, can also be used as primers in the present method. They can be used at concentrations that have been shown to induce a change of cell function (Cohen et al., 1999). Priming agents can be used alone or in combination. For example, one or more microtubule inhibitors may be used alone or together, or in combination with one or more microfilament inhibitors (Shea, 1990). A combination of both microfilament and microtubule inhibitors, at experimentally determined concentrations, can be used to effect complete destabilization of the cytoskeleton.

The age of the donor providing fibroblasts may be another factor in determining “priming” response. Fibroblasts from younger patients may display higher “priming” potential than fibroblasts from older patients and will be examined in initial experiments. Nuclear transfer (NT) experiments in animals indicate that cells derived from younger donors reprogram better and result in higher proportions of NT embryos that complete prenatal development than do embryos created from adult somatic cells (Yang et al., 2000).

The extent of priming itself may prove to be limiting. This could be due to cells' inability to erase nuclear memory to the extent that is required for a change in function. Similarly, donor cells obtained from one cell lineage (i.e. ectoderm) may only be primed to develop into another ectoderm derived cell type. To overcome this potential pitfall, cells will be conditionally immortalized/transformed. Transformed, immortalized cells that can commonly be found in various types of cancer have been shown to be multipotential and can be viewed as “primed” cells. Conditional imortalization of cultured primary cells may be accomplished by transfection with a transgene expressing a mutant, heat labile, form of the SV40 Large T antigen (Bond et al., 1996; SV40tsA58). Cells transgenic for this antigen can be immortalized by culture at 33 degrees C., where the Large T antigen is intact and biologically active. The cells can than be returned to a primary functional state by increasing the incubation temperature to 37 degrees C., where the antigen is truncated and not active at this higher temperature. Since immortalized cells display qualities of de-differentiated cells, they may be more easily primed, then induced to differentiate by supplying the appropriate culture conditions for the desired cell type. At the same time differentiation is induced, the cells can be returned to the non-immortalized state by raising the temperature. This strategy will be employed if difficulty arises in the transdifferentiation of primary cultures (above). Ultimately, if this approach proves to be viable, then the transgene will be flanked with loxp sites, so that it can be removed from the final product using Cre recombinase. We will attempt to induce donor cells to acquire cancer-like characteristics first, and expose them to priming and/or induced differentiation (Cohen et al., 1999).

A second approach to enhancing priming involves manipulation of nuclear structure with drugs that interfere with acetylation and/or methylation. There is a wealth of published literature describing the beneficial effects of deacetylase inhibitors (trichostatin A; Yoshida et al., 1995) and methylase inhibitors (5-aza-cytidine; Boukamp, 1995) on permissiveness of nuclear chromatin for transcription factors, transcription enhancers and other proteins involved in genomic transcription (Kikyo and Wolffe, 2000). Combined use of agents that interfere with acetylation and/or methylation and agents that disrupt the cytoskeleton may allow for shorter priming incubations, more complete reversal of nuclear function and therefore increase the range of cells that can be derived from primed cell populations. Donor cells of choice should have a stable karyotype, have to be able to support expansion in vitro, and survive cryopreservation and subsequent thawing. Some cell types may be better suited for this purpose than others. Also, the long-term effect of ploidy changes induced in trans-differentiated cell will have to be addressed.

2. Utilizing Methods that Effect Induction of Stem Cell Differentiation to Effect Trans-Differentiation of Primed Cells.

Conditions for driving embryonic stem and adult stem cells into several terminally differentiated phenotypes have been described (Bain et al., 1995; Pittenger et al., 1999; Fuchs and Segre, 2000; Lee et al., 2000; Bjornson et al., 2000; Schuldiner et al., 2000; Brustle et al., 1999). Even though we believe “priming” will not turn somatic cells into any type of stem cell, culture conditions that support differentiation of stem cells can be used to support differentiation of “primed” cells. One of the simplest, and best-documented differentiation protocols involves the use of retinoic acid to induce differentiation to neuronal cell precursors. Obtaining differentiated cells of the CNS (e.g. dopaminergic neurons, astrocytes, oligodendrocytes) is a good first step in testing the potency of primed cells, not only because it is the most direct method of obtaining differentiated cells, but also due to the size of the commercial markets for neuronal cell types in the treatment of Parkinson's Disease, Huntington's Disease, Alzheimer's Disease, multiple sclerosis, and repair of spinal cord injury.

Protocols developed for induction of neuronal precursors in mouse ES cells and human neuronal stem cells can be used for inducing the trans-differentiation of primed fibroblasts: serum-free medium, supplemented with retinoic acid, 5 mM ascorbic acid, bFGF2, PDGF on fibrinogen coated culture dishes. All cultures can be maintained in low oxygen environment (2-5%) and 5% CO₂ at 36.8 degrees C., as it has been shown that reducing O₂ concentration during cell culture dramatically increases the proportion of neuronal precursors that differentiate into dopaminergic neurons (15 to 56%; L. Studer, personal communication). Simultaneously, primed cells can be grown in culture conditions that have been described to support hematopoietic and muscle differentiation pathways (reviewed in Fuchs and Segre, 2000). Cells can be examined for their morphology by time-lapse video imaging and induction of expected gene and protein expression by RT-PCR and ICC, respectively.

TABLE 5
Initial inducing culture conditions for primed cells and expected
gene markers.

| Expected | | |
|---|---|---|
| outcome | culture conditions | induction markers |
| Neuronal | bFGF, FGF8, SHH, | TH, Nurr-1, Pax 3, 5, 8, En-1, |
| | EGF, PDGF, T3, CNTF | FGFR3, GDNF, TUJ1, CalR |
| | | 4B3, SMP |
| Hematopoietic | RPMI-40, interleukins, | CD14, CD34, CD45 |
| | GM-CSF, M-CSF, | |
| | G-CSF, erythropoietin, | |
| | thrombopoietin | |
| Muscle | BMP-2 | myoD1, skeletal myosin LC, |
| | | cardiac actin, desmin smooth |
| | | muscle actin |

Dopamine release can be induced as described (Cibelli et al., 2001). Briefly, culture medium is removed and replaced with Ca-free, Mg-free HBSS. After 15 minutes, this medium is replaced with Ca-free, Mg-free HBSS, supplemented with 56 mM KCl and samples of medium collected after 15-20 minute incubation and stored at −80 degrees C. until assayed.

Data Collection and Analysis: Control, non-primed cells can be grown under the same culture conditions and assayed for both, down-regulation of endogenous genes and proteins, as well as expression of genes induced by culture conditions. The assay for dopamine can be performed by HPLC as described elsewhere. Samples collected prior to KCl induced release can be used for control measurements. In addition to dopamine, the samples can be assayed routinely for serotonin, acetylcholin, and GABA.

Cell type-designed culture conditions will yield cells resembling the expected cell type. Neuronal cell types show induction of gene and protein markers described above. For example, Neurons secrete neuro-transmitters in a time dependent manner that correlates with cell morphology. If required, electrophysiology experiments can be designed to test excitability. Control cells are expected to retain their original phenotype, maintain the corresponding gene and protein expression and show absence of non-specific gene and protein expression. Sufficient cell numbers are available for these analyses since virtually all the primary cells respond to priming, and therefore their numbers can be manipulated by expansion prior to priming.

The gene expression profile specific only to the donor cells is turned off during priming without reversal into a stem cell-like state. In addition, during trans-differentiation, only expression of specific genes corresponding to the predicted types of trans-differentiated cells is turned on.

3. Combinations of Agents Acting on Intracellular Components and Extracellular Matrix for Reproducible Induction of a Single Cell Type.

Characterization of the type of cell being formed is an aspect of the present method. The method permits analysis and definition of all of the conditions that enable production of functional neurons from fibroblasts. It is useful to determine whether neurons are being produced in a subset of the total population of induced cells. It is known from induction of embryonic stem cells that primarily certain cell types are produced using specific growth factors (GFs) or cytokines. However, these populations are not pure and other cell types persist. Animal serum contains a plethora of proteins and peptides of undefined quantities. Thus, serum contains growth factors and cytokines that support growth and differentiation of essentially all cell types in the body. Therefore, serum-free culture conditions can be developed in order to properly evaluate the effect of specific combinations of GFs and cytokines on differentiation of primed cells. In addition, the effect of various artificial extracellular matrices (ECM) can be tested. The serum-free culture conditions do not necessarily need to induce proliferation but must sustain viability of the cells in vitro. The specific type of culture surface can also be evaluated. Whenever available, human versions of the required growth factors can be used, since the activity of many cytokines is not always equivalent across species.

Due to the human genome project, most of the GFs commercially available are from recombinant human genes. First, primary cell cultures are gradually adapted to serum-free conditions. Then, priming is induced by conditions discussed above. Primed cells in serum-free conditions can be subjected to culture conditions that yield or support specific neural cell types. Growth factors/cytokines that can be used include bFGF, FGF8, SHH, EFG, PDGF, T3, and CNTF. The cell culture surface and ECM materials that can be used include tissue culture plastic, bacterial culture plastic, glass, methylcellulose, fibrinogen, fibronectin, gelatin, collagen, laminin, poly-L-lysine, and poly-L-ornithine. The effect of a selected single GF in combination with a single ECM substrate can be evaluated to optimize conditions. Cells can be assayed for the presence of critical markers for specific cell types using ICC: astrocytes (GFAP), oligodendrocytes (O4), and neurons (TH). The cells produced from induction into neurons can be further assayed for dopamine, serotonin, acetylcholine, and GABA release. Once the interaction between individual growth factor/cytokine and ECM that leads to enrichment of specific neuronal cells types is determined, combinations of GF's/cytokines with the optimal ECM can be evaluated. The GF/cytokine and ECM combination result that leads to the purest population of dopaminergic neurons can thus be determined experimentally.

Gene expression at the RNA level can be determined by RT-PCR and translation products assayed by immunocytochemistry and/or Western blotting. Markers for the expression of specific genes in the differentiated state can be identified depending on the cell type. Immunocytochemistry can also be used to determine the purity of the cell population. RT-PCR primers and hybridization probes and antibodies for ICC and Western blotting are commercially available. Quantitative analysis of gene expression can be analyzed by Northern blots. Temporal changes in morphology can be recorded at regular intervals using time-lapse video imaging. Expression of key marker genes can be monitored at experimentally determined time points to evaluate the timing of priming and differentiation events. This approach yields information as to how long it takes for the donor somatic cell to become responsive to new signals and how long differentiation takes for various cell types.

By the methods described above, a combination of GFs/cytokines and ECM that yields predominantly specific neural cell types can be identified. For example, optimal conditions that yield dopaminergic neurons can be identified. In addition to generation of desired cell types by designed differentiation protocols, undesired cell types may result. Specific growth factor and cytokine combinations may result in an array of cell types, which it may be necessary to characterize. Three-dimensional factorial design of experiments (cytokine×growth factor×matrix) may be performed in conjunction with development of a comprehensive database for tracking cell response. Construction of a reasonably informative database includes catalogued information on donor cell type, primer, priming conditions, timing of gene/protein down regulation, a list of these genes/proteins, induction components, timing and expression of trans-differentiated cell type-specific genes/proteins, a list of these genes and proteins, cell survival and secretory properties (if any).

If cells trans-differentiate into more than one cell type, single cell cloning may be used to generate pure cell populations. It is well established that single cell culture is challenging and many cells do not survive in vitro on their own. Efforts should therefore be made to develop single cell cultures that keep cells physically separated while maintaining the same culture environment. Trans-differentiated cells may have an altered life span. Whether the lifespan is shortened or lengthened can be determined by a longevity analysis, which is routinely performed. If trans-differentiated cells display a shorter lifespan than control donor cells, lifespan can be maintained by reducing O₂ concentration during culture to <2%, designing shorter priming protocols, or avoiding excessive in vitro proliferation of donor cells prior to priming.

In addition to the foregoing, injection of primed cells into a live model (mouse) into sites that promote certain cell types can also be performed as a means for effecting trans-differentiation of primed cells.

Finally, trans-differentiation of primed cells can be effected by culturing the primed cells in the presence of other cells that are capable of inducing their neighbors to express specific markers due to paracrine effects. For example, it has been shown that cells transgenic for Pax-8 cause neighboring cells to become dopaminergic neurons (L. Studer, personal communication).

4. Maintain Stable Morphology and Function of Newly Differentiated Cells.

In order for newly differentiated cells to be useful for cell therapy, they must not only attain desired cell shape and function in vitro but also be able to maintain the newly established phenotype/function after trans-differentiation. The maintenance of a stable cell phenotype can be achieved by terminally arresting the cell cycle in G₀, an event that is induced in vivo by differentiation itself. While trans-differentiated cells may retain certain nuclear plasticity, appropriate conditions in vitro or in vivo should allow for stabilization of their phenotype. Maintaining the same environmental signals (same medium, same supplemental factors, temperature, and matrix conditions) stabilizes cell phenotype.

Newly trans-differentiated cells can be cultured continuously and monitored at specific time points for expression of cell type-specific markers. Culture occurs in the absence of “priming” agent and under conditions consistent with the “new” cell type. In addition, the cells can be grown in media (or conditions) that are not consistent with the new cell type to evaluate stability. Of particular importance will be the behavior of newly trans-differentiated cells in culture conditions specific for the original donor cell type.

Data Collection and Analysis: Morphology of induced cell can be monitored and progression recorded by video imaging. Gene expression and protein expression/localization can be evaluated by RT-PCR and ICC, respectively for neuronal antigens (neurofilament, enolase, tyrosin hydroxylase, GFAP, dopamine receptor, myelin), muscle specific antigens (.beta.-actin, desmin, myosin heavy chain), and hematopoietic cell markers (CD34).

After withdrawal of the priming agent (e.g., microfilament inhibitor), the cells retain their newly acquired phenotype and either re-enter the cell cycle or remain arrested in G₀, depending on the cell phenotype. New neurons are expected to remain in G₀ and not proliferate, retain neuronal morphology, secrete neurotransmifters, establish synapses and remain viable for up to 4 days in vitro (Lorenz Studer, personal communication).

It may be challenging to keep population of cells pure since this is not how they grow in vivo. To maintain a stable function in vivo, cells have to interact with their neighboring cells that are of often of a different phenotype (e.g. neurons with glia, muscle with connective tissue and vascular endothelium, etc). It may be necessary to grow new cell types under one of the following two conditions. (1) Growth on a three-dimensional matrix (3-D). This will allow them to establish a more physiological 3-D structure, initiate spatial interactions and start producing their own extracellular matrix. This strategy will be exploited during induction of differentiation. (2) Grow newly differentiated cells on monolayers of cell types with which they would interact normally in vivo.

5. Evaluating Trans-Differentiated Cells for Therapeutic Efficacy by In Vivo Cell Transplantation into an Animal Model.

In vivo function of neural cells generated via trans-differentiation from somatic cells is crucial for evaluating therapeutic potential. Several standardized test have been developed in rodents that can reliably mimic clinical symptoms of specific neurological disorders such as Parkinson's disease, Huntington's disease, spinal cord injury, epilepsy or stroke. Transplantation of neurons derived from the developing CNS can significantly improve clinical symptoms in many of these animal models. Cell therapy is especially promising in Parkinson's disease where a relatively small and well-defined population of specific neurons is lost. Clinical transplantation of fetal dopamine neurons has been performed in over 300 patients worldwide and long-term benefit has been demonstrated in patients for at least up to 10 years after transplantation (Piccini et al. 1999). More recently, encouraging results have also been reported for fetal tissue grafting in Huntington's disease (Bachoud-Levi et al. 2000). However, the use of fetal tissue raises significant ethical and technical concerns that have prevented more widespread use of the technology (Freeman et al. 2000). The availability of an easily accessible and renewable source of neural cells will dramatically improve the technical and social outlook of CNS cell transplantation in neurodegenerative disorders. The availability of such a cell source might also obviate the use of immunosuppression in subjects undergoing CNS transplantation and alleviate some of the ethical and psychological concerns of implanting brain cells derived from another individual or species as in the case of fetal pig dopamine neurons (Deacon et al. 1997).

Experimental: Parkinsonian rats and mice are created by unilateral stereotactic injection of the neurotoxin 6-OHDA that is taken up specifically by dopaminergic terminals and retrogradely transported to the cell body where it induces apoptotic cell death. The behavioral outcome of the transplanted cells is assessed using state of the art behavioral tests including rotometer assays. Upon stimulation with drugs that mimic dopamine effects Parkinsonian animals show an asymmetric behavior with postural asymmetry, ipsilateral rotation and contra lateral hemineglect. Animals undergo repeated behavioral tests 2-4 weeks after 6-OHDA injection. Animals with stable behavioral deficits are randomly selected for cell implantation or control group (12 animals each, controls: injection of non-dopaminergic cell or saline). Cells are tested for dopamine production prior to transplantation using non-invasive measurements of dopamine release (Studer et al. 1996; Studer et al. 1998). Upon intrastriatal implantation of functional dopamine neurons Parkinsonian symptoms such as rotation behavior should gradually disappear within a period of 4-16 weeks. After completing the behavioral studies the animals are perfused with paraformaldehyde and the brains subjected to immunohistochemical analyses (Studer et al. 1998). Surviving dopamine neurons in the host striatum are identified by immunohistochemistry for tyrosine-hydroxylase, the rate-limiting enzyme in the synthesis of dopamine. Quantification of cell numbers are performed using stereology-based computer assisted counting procedures.

Data Collection and Analysis: Surgical data: We have described our procedures for inducing neurodegenerative lesions as well as performing cell transplantation been described previously in great detail (Tabar and Studer 1997). A hierarchical computer database linked to behavioral and histological results is set up to record all relevant data for each animal included in the study. Behavioral data: Rotation data is collected on a commercially available rotometer system (San Diego Instruments). ASCII files are imported into statistical software for further analyses (Microsoft Excel and Statistica, Statsoft). In vitro functional testing prior to transplantation: Dopamine and serotonin production of the cells to be grafted are assayed using reverse-phase HPLC with electrochemical detection as described previously (Studer et al. 1998; Studer et al. 1996). Data is collected on ESA proprietary software and exported to Statistica (Statsoft) for further analyses. Histological analyses: The number of surviving dopamine neurons in the grafted brain are assessed using stereological counts of Tyrosine-hydroxylase (TH+) cells in the striatum (Studer et al. 1998; Gundersen et al. 1988).

Expected Results: Establishing Parkinsonian lesions in rodents: Typically about 60-80% of the animals subjected to stereotactic 6-OHDA injections show a stable rotation response upon amphetamine injection three weeks after surgery. Recovery of lost function depends on the number and function of the grafted cells. It has been established in fetal tissue grafts that about 1000 rodent dopamine neurons are required to completely restore the rotation behavior of 6-OHDA rodents. The survival rate of dopamine neurons is typically about 5-10%.

Potential Difficulties, Limitations and Alternatives: Animal model: The mouse or rat strain has to be chosen carefully as certain strains show hypersensitivity to some anesthetics such as barbiturates or in some cases various sensitivities to the neurotoxic drugs used. An alternative strain and adaptation in the dose of the neurotoxic drugs would be required. Behavioral test: The degree of Parkinsonian symptoms can vary among animals. Especially in mice the success rate of inducing a stable Parkinsonian lesion is lower and spontaneous recovery has been reported. An alternative strain or neurotoxin can be utilized in such cases. Histology: No difficulties are to be expected if state-of-the-art technical procedures are followed. Alternative disease model: The generation of specific dopamine neurons is challenging. Only about 1:10̂4-1:10̂5 of all neurons in the adult brain are midbrain dopamine neurons (Hynes and Rosenthal 2000). If no dopamine but other neuronal subtypes are available for grafting, an alternative disease model will be chosen such as ibotenic acid lesion in rodents to mimic Huntington's disease (Tabar and Studer 1997) with subsequent transplantation of neurons exhibiting the more common neurotransmitter GABA.

Reprogramming Animal Somatic Cells

Introduction

Advances in stem cell technology, such as the isolation and use of human embryonic stem (hES) cells, have become an important new subject of medical research. hES cells have a demonstrated potential to differentiate into any and all of the cell types in the human body, including complex tissues. This ability of hES cells has led to the suggestion that many diseases resulting from the dysfunction of cells may be amenable to treatment by the administration of hES-derived cells of various differentiated types (Thomson et al., Science 282:1145-7, (1998)). Nuclear transfer studies have demonstrated that it is possible to transform a somatic differentiated cell back to a totipotent state such as that of ES or ED cells (Cibelli et al., Nature Biotech 16:642-646, (1998)). The development of technologies to reprogram somatic cells back to a totipotent ES cell state such as by nuclear transfer offers a means to deliver ES-derived somatic cells with a nuclear genotype of the patient (Lanza et al., Nature Medicine 5:975-977, (1999)). It is expected that such cells and tissues would not be rejected, despite the presence of allogeneic mitochondria (Lanza et al., Nature Biotech 20:689-696, (2002)). Nuclear transfer also allows the rebuilding of telomere repeat length in cells through the reactivation of the telomerase catalytic component in the early embryo (Lanza et al., Science 288:665-669, (2000)). Nevertheless, there remains a need for improvements in methods to reprogram animal cells that increase the frequency of successful and complete reprogramming and reduce the dependence on the availability of human oocytes.

Because of the relative difficulty of obtaining large numbers of human oocytes, there has been considerable interest in determining whether other germ-line cells, such as cultured ES cells, or cytoplasm from said cells, could be used to reprogram somatic cells. Such cells would have an important advantage over oocytes as a means of inducing reprogramming in that they can be easily expanded in number in vitro. The restoration of expression of at least some measured embryonic-specific genes has been observed in somatic cells following fusion with ES cells (Do and Scholer, Stem Cells 22:941-949, (2004); Do and Scholer, Reprod. Fertil. Dev. 17:143-149, (2005)). However, the resulting cells are hybrids, often with a tetraploid genotype, and therefore not suited as normal or histocompatible cells for transplant purposes. Indeed, one of the proposed purposes of generating autologous totipotent cells is to prevent the rejection of ES-derived cells. Using the techniques described in these published studies, the ES cells used to reprogram a patient's cell would therefore likely add alleles that could generate an immune response leading to rejection. Nevertheless, the evidence that ES cells can reprogram somatic cell chromosomes has excited researchers and generated a new field of research called “fusion biology” (Dennis, Nature 426:490-491, (2003)). Another potential source of cells capable of reprogramming human somatic cells with a greater ease of availability than human oocytes are oocytes of animal species. The demonstration of the restoration of totipotency in somatic cells by nuclear transfer across species (Lanza et al., Cloning 2:79-90, (2000)) opens the possibility of identifying animal oocytes that can be easily obtained for use in reprogramming human cells (Byrne et al., Curr Biol 13:1206-1213, (2003)). However, likely because of molecular differences between the species, cross species nuclear transfer, although possible, is often even more inefficient than same-species nuclear transfer. Among the many molecular alterations that occur following somatic cell nuclear transfer, some of the more critical alterations are the reprogramming of the chromatin through poorly-understood mechanisms in the recipient oocyte and remodeling of the proteins of the nuclear envelope. The nuclear envelope includes the inner nuclear membrane (INM) and outer nuclear membrane (ONM), nuclear pore complexes (NPCs), and nuclear lamina. The proteins of the nuclear envelope, in particular those proteins of the lamina, differ between somatic and germ-line cells and play an important role in regulating the cell cycle, monitoring DNA damage checkpoint pathways, and regulating cell differentiation. In particular, the protein subunits of the lamina include the type V intermediate filament proteins, lamin A/C and B, which form a meshwork internal to the INM (Foisner, J. Cell Sci. 114:3791-3792, (2001)). Some of these proteins, such as lamin A/C, play an important role in regulating chromosomal integrity, DNA damage checkpoints, and telomere status signaling through their interactions with the WRN helicase, POT1, Tel1, and Tel2. In germ-line cells that are telomerase positive, or where telomerase is utilized, the nuclear matrix lacks lamin A/C or otherwise allows tandemly-repeated sequences of DNA to be repaired and, in the case of the telomere, to be lengthened by telomerase. Other proteins associated with the INM include the family of lamina associated polypeptides (LAPs) including lamina-associated protein 1 (LAP1, of which there are at least three isoforms (α, β, and Y)), LAP2 (with at least six isoforms) and emerin (which when mutated leads to abnormal muscle differentiation and Emery-Dreifuss muscular dystrophy). Other proteins associated with the INM include the ring finger binding protein (RFBP), otefin, germ cell-less (GCL) and nurim. The lamins are known to play an important role in regulating the function of transcriptional regulators such as the retinoblastoma protein (pRB) and LBR which in turn can bond heterochromatin protein 1 (HP1). By way of example of the need to remodel the nuclear envelope in order to reprogram a differentiated somatic cell to an undifferentiated state, undifferentiated germ-line cells generally lack the presence of lamin A, while germ-line cells contain proteins such as germ cell-less (GCL) and lamin C2, which are often not expressed in differentiated somatic cells (Furukawa et al., Exp. Cell Res. 212:426-430, 1994). Incomplete remodeling of the nuclear envelope would contribute to the inefficiency or incomplete reprogramming of cells using existing technologies.

Therefore, each of the technologies to reprogram human somatic cells known in the art have their own unique difficulties. SCNT provides a satisfactory level of reprogramming but is limited by the number of human oocytes available to researchers. Cross-species nuclear transfer and cell fusion technologies are not generally limited in the cells used in reprogramming but are limited by the degree of successful reprogramming or the robustness of the growth of the resulting reprogrammed cells. Therefore, there remains a need for improved technologies to both increase the frequency and quality of reprogramming of differentiated somatic cells and of producing reprogrammed cells that are capable of expansion in vitro in order to obtain a useful number of cells for research, testing for quality control, and for use in cell therapy. The present method combines aspects of several existing technologies already known in the art in a novel and non-obvious manner to provide a means of reprogramming differentiated cells as effectively or more effectively than SCNT and to provide a more acceptable and cost-effective substitute for oocytes as the vehicle for reprogramming. The present method achieves these goals in part by using cells that are easily and inexpensively obtained in unlimited quantities and a technology that can be scaled such that thousands or millions of fusions can be performed simultaneously, thereby increasingly the probability of a successful final outcome. Additionally, the present method provides a technique that facilitates the reactivation of telomerase and an extension of telomere length, thereby. restoring cell replicative lifespan. The present method further provides an assay that allows for the analysis of what components in undifferentiated and germ-line cells are critical for nuclear reprogramming. The method also provides a procedure that can be automated through robotics to reduce cost and improve quality control.

Methods of Reprogramming Animal Somatic Cells

The present section describes methods for the reprogramming of differentiated cells to a more pluripotent state by utilizing a multiple-step procedure that includes a distinct nuclear remodeling step and a cellular reconstitution step.

Step 1: Nuclear Remodeling

The method utilizes a three-step process to improve the efficiency of reprogramming differentiated cells to an undifferentiated state.

In the first step, designated the nuclear remodeling step, the nuclear envelope and the chromatin of a differentiated cell are remodeled to more closely resemble the molecular composition of the nuclear envelope and chromatin, respectively, of an undifferentiated or a germ-line cell. This remodeling step can be performed in numerous ways, but the unique and nonobvious feature of this method is that this remodeling step is performed in a separate step from the transfer of the remodeled genome into a cytoplast; further, the cytoplast is a cytoplast that is readily available, such as nonhuman animal oocyte cytoplasts or cytoplasts prepared from embryonal carcinoma (EC) cell lines, including EC cell lines genetically modified to make extracts and cytoplasts with improved capacity to reprogram under the present method and that will then yield the final proliferating cell types. The remodeling of the somatic cell nucleus could be performed by transferring the nucleus into an oocyte of the same species (though differing in genotype from that somatic cell) or into an oocyte of a different species such as fish or amphibian (e.g. Xenopus) oocyte or egg, or in dispersed extracts from cells capable of reconstituting an undifferentiated or germ-line nuclear envelope around what was originally a genome from a differentiated cell.

Separating the nuclear remodeling step from the cellular reconstitution step solves problems inherent in existing reprogramming technologies. If nuclear remodeling is performed in one step separate from the step of cellular reconstitution to generate cells capable of proliferation, then it is possible to eliminate a dependence on oocytes of the same species as the differentiated cell and increase efficiency.

In the case of SCNT, the oocyte is a relatively large cell and as a result when a differentiated cell is transferred into a metaphase II oocyte, the ensuing breakdown of the nuclear envelope and chromosome condensation, and reassembly of the nuclear envelope largely from egg cell-derived components, results in the formation of a remodeled nuclear envelope as well as the impartation of nuclear regulatory factors, such as transcription factors, useful in reprogramming the chromatin. If the egg cell is activated at about the time of nuclear transfer, cell division may also occur, resulting in an embryo capable of giving rise to a culture of ES cells. The problems inherent in nuclear transfer, however, are that despite the relatively large volume of the oocyte and the incorporation of oocyte cell nuclear components into the reconstructed cell, nuclear transfer requires micromanipulation, which is a highly-skilled procedure, as well as serial production using one cell at a time. Further, nuclear transfer is limited by the number of oocytes available. In the present method, these difficulties are addressed by utilizing alternative nuclear remodeling technologies that, although requiring more than one step to obtain intact cells capable of cell division, nevertheless allow easy access to cytoplasm and are capable of remodeling a nucleus. Furthermore, these alternative techniques allow the simultaneous remodeling of many nuclei or genomes.

One modality for performing the first step of nuclear remodeling is through the use of fish or amphibian oocytes. The oocytes or eggs from the species Xenopus laevis have the advantage that they are widely studied, though most other oocytes or eggs from vertebrate species will function in a similar manner with the exception of egg cells with a large amount of yolk. While Xenopus oocytes are only marginally useful in reprogramming the chromatin of mammalian differentiated cell nuclei (Byrne et al., Curr Biol 13:1206-1213, (2003)), they can be used to nearly completely reassemble a germ-line nuclear envelope around a large number of differentiated somatic cells. Using Xenopus oocytes or Xenopus oocyte extract, the nuclear envelope and chromatin of the somatic cell is remodeled in the presence of such undifferentiated or germ-line proteins through a variety of means, including the injection of one or more intact or permeabilized differentiated cells into the oocyte, or the injection of isolated nuclei from said cells, into an oocyte. Further, other undifferentiated protein or other factors may be added to the oocytes or oocyte extract, or oocytes may be modified to express such additional factors that facilitate nuclear remodeling.

The differentiated cell that is reprogrammed may be any differentiated cell of a vertebrate species such as human, canine, equine, or feline somatic cells including fibroblasts, keratinocytes, lymphocytes, monocytes, epithelial cells, hematopoietic cells, or other cells.

One protocol for remodeling the nuclear envelope of these differentiated cells using oocytes from another species, such as Xenopus oocytes, is to inject permeabilized differentiated cells into interphase Xenopus oocytes, thereby allowing multiple differentiated cell nuclear envelopes to be remodeled over a period of several days. Xenopus oocytes from anesthetized mature females are surgically removed in MBS (magnesium buffered saline) and inspected for quality as is well-known in the art (Gurdon, Methods Cell Biol 16:125-139, (1977)). The oocytes are then washed twice in MBS and stored overnight at 14° C. in MBS. The next day, good quality stage V or VI oocytes are selected (Dumont, J. Morphol. 136:153-179, (1972)) and follicular cells are removed under a dissecting microscope in MBS. After defolliculation, the oocytes are stored again at 14 degrees C. overnight in MBS with 1 μg/mL gentamycin (Sigma). The next day, oocytes with a healthy morphology are washed again in MBS and stored in MBS at 14 degrees C. until use that day. The differentiated cells are then permeabilized by a permeabilization agent, such as Streptolysin O (SLO) or digitonin (Chan & Gurdon, Int. J. Dev. Biol. 40:441-451, (1996); Adam et al., Methods Enzymol. 219:97-110, (1992)). Approximately 1×10̂4 differentiated cells are permeabilized, and suspended in ice-cold lysis buffer (1×Ca2+-free MBS containing 10 mM EGTA (Gurdon, (1977)]. SLO (Wellcome diagnostics) is added at a final concentration of 0.5 units/mL. The suspension is maintained on ice for 7 minutes, then four volumes of 1×Ca2+-free MBS containing 1% bovine serum albumin (Sigma) is added. Aliquots of the cells may then be removed, diluted 1× in 1×Ca2+-free MBS containing 1% bovine serum albumin, and incubated at room temperature for five minutes to activate permeabilization. The cells are then placed back on ice for transfer into the Xenopus oocytes. The permeabilized cells are then transferred into Xenopus oocytes as is well known in the art (Gurdon, J. Embryol. Exp. Morphol. 36:523-540, (1976). Briefly, oocytes prepared as described above are placed on agar in high salt MBS (Gurdon, J. Embryol. Exp. Morphol. 36:523-540, (1976)). The DNA in the egg cells is inactivated by UV as described (Gurdon, Methods in Cell Biol 16:125-139, 1977) with the exception that the second exposure to the Hanovia UV source is not performed. Briefly, egg cells are placed on a glass slide with the animal pole facing up and are exposed to a Mineralite UV lamp for 1 minute to inactivate the female germinal vesicle. The permeabilized differentiated cells are taken up serially into a transplantation pipette 3-5 times the diameter of the cells and injected into the oocyte, preferably aiming toward the inactivated pronucleus. The egg containing the nuclei are incubated for one hour to 7 days and the nuclei are then removed and cryopreserved or used immediately in step two to reconstitute cells capable of proliferation.

Another manner in which the nuclear envelope and chromatin are remodeled is in cell-free extracts capable of forming nuclear envelopes from naked DNA or chromatin. Techniques for assembling nuclear envelopes around DNA or chromatin are known in the art (Marshall & Wilson, Trends in Cell Biol 7:69-74, (1997)). Such extracts may be isolated, for example, from Xenopus oocytes as is well-known in the art (Lohka, Cell Biol Int. Rep. 12:833-848 (1988)). Alternatively, extracts from undifferentiated cells of the same species may be used such as Mil oocytes, oocytes at other stages of development, ES cells, EC cells, EG cells, or other cells in a relatively undifferentiated state. EC cells provide the advantage that they can be easily propagated in large quantities and human rather than nonhuman EC cells lessen concerns over the transmission of uncharacterized pathogens. Nonlimiting examples of such human EC cells include NTera-2, NTera-2 Cl. Dl, NCCIT, Cates-1B, Tera-1, AND TERA-2 and nonlimiting examples of murine EC lines include MPRO, EML, F9, F19, Dl ORL UVA, NFPE, NF-1, and PFHR9. EC lines are readily obtained from sources such as the American Type Culture Collection and are grown at 37 degrees C. in monolayer culture in medium characterized for that cell type and readily available on the internet, (http://stemcells.atcc.org) (complete medium).

In certain embodiments, the genome of the remodeled nucleus may be modified. Such modifications include, but are not limited to, the correction of mutations affecting disease, and other genetic modifications that alleviate disease symptoms or causes (e.g., in genes that would otherwise be targeted or used in gene-therapy). The nucleus being remodeled in step one may be modified by the addition of extracts from cells such as DT40 known to have a high level of homologous recombination. The addition of DNA targeting constructs and the extracts from cells permissive for a high level of homologous recombination

will then yield cells after reconstitution in step 2 and screening in step 3 that have a desired genetic modification. For example, in certain embodiments, reprogrammed cells may be used to generate cells or tissues for cell-based therapies and/or transplantation.

In other embodiments, one or more factors are expressed or overexpressed in the undifferentiated cells (for example, in EC cells) used to obtain the nuclear remodeling extract or one or more factors may be added to the undifferentiated cells. Such factors include, for example, SOX2, NANOG, cMYC, OCT4, DNMT3B, embryonic histones, as well as other factors listed in Table 7 and their non-human counterparts. Increased expression of these factors may confer characteristics of an undifferentiated cell to the somatic cell nuclei and/or remove differentiated cell factors, thereby improving the frequency of reprogramming. Accordingly the method also may include adding, expressing or over-expressing any other proteins that confer characteristics of an undifferentiated cell. In addition to the proteins mentioned above, the present method may include other factors (such as transcriptional regulators and regulatory RNA) that induce or increase the expression of proteins expressed in undifferentiated cells and that improve the frequency of reprogramming. Further, any combinations of the above-mentioned factors may be used. For example, undifferentiated cells of the present method may be modified to have increased expression of two, three, four, or more of any of the factors listed in Table 7. Likewise, two, three, four, or more of any of the factors listed in Table 7 may be added to the remodeling extract.

In other embodiments, the level of one or more factors in the undifferentiated cells used to obtain the nuclear remodeling extract is decreased relative to the levels found in unmodified cells. Such decreases in the level of a cell factor may be achieved by known methods, such as, for example, by use of transcriptional regulators, regulatory RNA, or antibodies specific for the cell factor.

In certain embodiments, gene constructs encoding the proteins listed in Table 7 or other factors, or regulatory proteins or RNAs that induce expression of these factors, are transfected into the cells by standard techniques. Such techniques include viral infection (e.g., lentivirus, papilloma virus, adenovirus, etc.) and transfection of plasmid and other vectors by chemical transfection (e.g., via calcium phosphate, lipids, dendrimers, etc.), electroporation, and microinjection. Alternatively, constructs that target the factors' endogenous promoters may be used to induce or increase expression of the factors. Other embodiments may use artificial chromosomes comprising one or more of these factors. In additional embodiments, chromosome mediated gene transfer or cell fusion/microcell fusion are used to introduce these factors into an undifferentiated cell. In other embodiments, homologous recombination to modify gene regulatory sequences can achieve increased expression of one or more of these factors.

In some embodiments, a transgene encoding the cell factor of interest may be delivered to the cell by pronuclear microinjection of DNA that is coated with recombinase. See, for example, Maga et al., Transgenic Research 12:485-496 (2003). Other known methods to improve the efficiency of generating transgenic cells may likewise be useful for purposes of this method. Alternatively, the oocytes and/or undifferentiated cell extracts of the present method may be obtained from transgenic animals that express human reprogramming factors (such as the factors listed in Table 7). For example, transgenic animals are generated using expression constructs carrying one or more of the genes listed in Table 7.

In some embodiments, the cell factors, or agents that alter the intracellular levels of the cell factors, may be introduced into undifferentiated cells by direct intracellular delivery. For example, the factors may be delivered using protein transduction domains or cell penetrating peptides, such as, for example, polyarginine. See Noguchi et al., Acta Med. Okayama 60:1-11 (2006). Cells into which the factors have been introduced may thus be useful in the above methods for nuclear remodeling.

In alternative embodiments, undifferentiated cell factors (such as the proteins and protein equivalents listed in Table 7), or agents that affect the levels of the cell factors, are introduced directly to the nuclear remodeling extract. In certain embodiments, recombinant proteins are added to the extract to improve the reprogramming efficiency.

The differentiated cells that may be effectively reprogrammed using the present method include differentiated cells of any kind from any vertebrate (including human), including without limitation skin fibroblasts, keratinocytes, mucosal epithelial cells, or peripheral nucleated blood cells, using the following steps.

Preparation of Nuclear Remodeling Extract

Extracts from germ-line cells, such as ES, EG, or EC cells including but not limited to NTera-2 cells, are prepared in the prometaphase as is known in the art (Burke & Gerace, Cell 44: 639-652, (1986)). Briefly, after two days and while still in a log growth state, the medium is replaced with 100 mL of complete medium containing 2 mM thymidine (which sequesters the cells in S phase). After 11 hours, the cells are rinsed once with 25 mL of complete medium, then incubated with 75 mL of complete medium for four hours, at which point nocodazole is added to a final concentration of 600 ng/mL from 10,000× stock solution in DMSO. After one hour, loosely-attached cells are removed by mitotic shakeoff (Tobey et al., J. Cell Physiol. 70:63-68, (1967)). This first collection of removed cells is discarded, the medium is replaced with 50 mL of complete medium also containing 600 ng/mL of nocodazole. Prometaphase cells are then collected by shakeoff 2-2.5 hours later. The collected cells are then incubated at 37 degrees C. for 45 minutes in 20 mL of complete medium containing 600 ng/mL nocodazole and 20 μM cytochalasin B. Following this incubation, the cells are washed twice with ice-cold Dulbecco's PBS, then once in KHM (78 mM KCl, 50 mM Hepes-KOH [pH 7.0], 4.0 mM MgCl2, 10 mM EGTA, 8.37 mM CaCl2, 1 mM DTT, 20 μM cytochalasin B). The cells are then centrifuged at 1000 g for five minutes, the supernatant discarded, and the cells are resuspended in the original volume of KHM. The cells are then homogenized in a dounce homogenizer on ice with about 25 strokes and progress determined by microscopic observation. When at least 95% of the cells are homogenized extracts held on ice for use in envelope reassembly or cryopreserved as is well known in the art.

Preparation of Condensed Chromatin from Differentiated Cells

Donor differentiated cells are exposed to conditions that remove the plasma membrane, resulting in the isolation of nuclei. These nuclei, in turn, are exposed to cell extracts that result in nuclear envelope dissolution and chromatin condensation. This dissolution and condensation results in the release of chromatin factors such as RNA, nuclear envelope proteins, and transcriptional regulators such as transcription factors that are deleterious to the reprogramming process. Differentiated cells are cultured in appropriate culture medium until they reach confluence. 1×10̂6 cells are then harvested by trypsinization as is well known in the art, the trypsin is inactivated, and the cells are suspended in 50 mL of phosphate buffered saline (PBS), pelleted by centrifuging the cells at 500 g for 10 minutes at 4° C., the PBS is discarded, and the cells are placed in 50× the volume of the pellet in ice-cold PBS, and centrifuged as above. Following this centrifugation, the supernatant is discarded and the pellet is resuspended in 50× the volume of the pellet of hypotonic buffer (10 mM HEPES, pH 7.5, 2 mM MgCl2, 25 mM KCl, 1 mM DTT, 10 μM aprotinin, 10 μM leupeptin, 10 μM pepstatin A, 10 μM soybean trypsin inhibitor, and 100 μM PMSF) and again centrifuged at 500 g for 10 min at 4 degrees C. The supernatant is discarded and 20× the volume of the pellet of hypotonic buffer is added and the cells are carefully resuspended and incubated on ice for an hour. The cells are then physically lysed using procedures well-known in the art. Briefly, 5 ml of the cell suspension is placed in a glass Dounce homogenizer and homogenized with 20 strokes. Cell lysis is monitored microscopically to observe the point where isolated and yet undamaged nuclei result. Sucrose is added to make a final concentration of 250 mM sucrose (1/8 volume of 2 M stock solution in hypotonic buffer). The solution is carefully mixed by gentle inversion and then centrifuged at 400 g at 4° C. for 30 minutes. The supernatant is discarded and the nuclei are then gently resuspended in 20 volumes of nuclear buffer (10 mM HEPES, pH 7.5, 2 mM MgCl2, 250 mM sucrose, 25 mM KCl, 1 mM DTT, 10 μM aprotinin, 10 μM leupeptin, 10 μM pepstatin A, 10 μM soybean trypsin inhibitor, and 100 μM PMSF). The nuclei are re-centrifuged as above and resuspended in 2× the volume of the pellet in nuclear buffer. The resulting nuclei may then be used directly in nuclear remodeling as described below or cryopreserved for future use.

Preparation of Condensation Extract

The condensation extract, when added to the isolated differentiated cell nuclei, will result in nuclear envelope breakdown and the condensation of chromatin. Because the purpose of step 1 is to remodel the nuclear components of a somatic differentiated cell with that of an undifferentiated cell, the condensation extract used is from undifferentiated cells which may or may not be also be the cells used to derive the extract for nuclear envelope reconstitution above. This results in a dilution of the components from the differentiated cell in extracts which contain the corresponding components desirable in reprogramming cells to an undifferentiated state. Germ-line cells such as ES, EG, or EC cells such as NTera-2 cl. Dl cells are easily obtained from sources such as the American Type Culture Collection and are grown at 37° C. in monolayer culture in appropriate medium (complete medium). While in a log growth state, the cells are plated at 5×10′6 cells per sq cm tissue culture flask in 200 mL of complete medium. Methods of obtaining extracts capable of inducing nuclear envelope breakdown and chromosome condensation are well known in the art (Collas et al., J. Cell Biol. 147:1167-1180, (1999)).

Briefly, the germ-line cells in log growth as described above are synchronized in mitosis by incubation in 1 μg/ml nocodazole for 20 hours. The cells that are in the mitotic phase of the cell cycle are detached by mitotic shakeoff. The harvested detached cells are centrifuged at 500 g for 10 minutes at 4 degrees C. Cells are resuspended in 50 ml of cold PBS, and centrifuged at 500 g for an additional 10 min. at 4° C. This PBS washing step is repeated once more. The cell pellet is then resuspended in 20 volumes of ice-cold cell lysis buffer (20 mM HEPES, pH 8.2, 5 mM MgCl2, 10 mM EDTA, 1 mM DTT, 10 μM aprotinin, 10 μM leupeptin, 10 μM pepstatin A, 10 μM soybean trypsin inhibitor, 100 μM PMSF, and 20 μg/ml cytochalasin B, and the cells are centrifuged at 800 g for 10 minutes at 4° C. The supernatant is discarded, and the cell pellet is carefully resuspended in one volume of cell lysis buffer. The cells are placed on ice for one hour then lysed with a Dounce homogenizer. Progress is monitored by microscopic analysis until over 90% of cells and cell nuclei are lysed. The resulting lysate is centrifuged at 15,000 g for 15 minutes at 4 degrees C. The tubes are then removed and immediately placed on ice. The supernatant is gently removed using a small caliber pipette tip, and the supernatant from several tubes is pooled on ice. If not used immediately, the extracts are immediately flash-frozen on liquid nitrogen and stored at −80° C. until use. The cell extract is then placed in an ultracentrifuge tube and centrifuged at 200,000 g for three hours at 4° C. to sediment nuclear membrane vesicles. The supernatant is then gently removed and placed in a tube on ice and used immediately to prepare condensed chromatin or cryopreserved as described above.

Methods of Use of Condensation Extract

If beginning with a frozen aliquot on condensation extract, the frozen extract is thawed on ice. Then an ATP-generating system is added to the extract such that the final concentrations are 1 mM ATP, 10 mM creatine phosphate, and 25 μg/ml creatine kinase. The nuclei isolated from the differentiated cells as described above are then added to the extract at 2,000 nuclei per 10 μl of extract, mixed gently, the incubated in a 37° C. water bath. The tube is removed occasionally to gently resuspend the cells by tapping on the tube. Extracts and cell sources vary in times for nuclear envelope breakdown and chromosome condensation. The progress is therefore monitored by periodic monitoring samples microscopically. When the majority of cells have lost their nuclear envelope and there is evidence of the beginning of chromosome condensation, the extract containing the condensing chromosome masses is placed in a centrifuge tube with an equal volume of 1 M sucrose solution in nuclear buffer. The chromatin masses are sedimented by centrifugation at 1,000 g for 20 minutes at 4 degrees C. The supernatant is discarded, and the chromatin masses are gently resuspended in nuclear remodeling extract derived above. The sample is then incubated in a water bath at 33° C. for up to two hours and periodically monitored microscopically for formation of remodeled nuclear envelopes around the condensed and remodeled chromatin as described (Burke & Gerace, Cell 44:639-652, (1986). Once a large percentage of chromatin has been encapsulated in nuclear envelopes, the remodeled nuclei may be used in cellular reconstitution using any of the techniques described below in step 2.

Step 2—Cellular Reconstitution

Step 2, also referred to as “cellular reconstitution” in the present method, is carried out using nuclei or chromatin remodeled by any of the techniques described in the present disclosure, such as in Examples 14 and 15 or combinations of the techniques described in Examples 14 and 15 as described more fully in the present disclosure.

One manner of performing step 2 using nuclei remodeled in step 1 of the present method is to fuse the remodeled nuclei with enucleated cytoplasts of germ-line cells such as blastomeres, morula cells, inner cell mass cells, ES cells (including hES cells, EG cells, and EC cells) as is known in the art (Do & Scholer, Stem Cells 22:941-949 (2004)). Briefly, the human ES Cells are cultured under standard conditions (Klimanskaya et al. Lancet 365: 4997 (1995)). The cytoplasmic volume of the cells is increased by adding 10 μM cytochalasin B for 20 hours prior to manipulation. Cytoplasts are prepared by centrifuging trypsinized cells through a Ficoll density gradient using a stock solution of autoclaved 50% (wt/vol) Ficoll-400 solution in water. The stock Ficoll 400 solution is diluted in DMEM and with a final concentration of 10 μM cytochalasin B. The cells are centrifuged through a gradient of 30%, 25%, 22%, 18%, and 15% Ficoll-400 solution at 36 degrees C. Layered on top is 0.5 mL of 12.5% Ficoll-400 solution with 10×10̂6 ES cells. The cells are centrifuged at 40,000 rpm at 36 degrees C. in an MLS-50 rotor for 30 minutes. The cytoplasts are collected from the 15% and 18% gradient regions marked on the tubes, rinsed in PBS, and mixed on a 1:1 ratio with remodeled nuclei from step one of the present method or cryopreserved. Fusion of the cytoplasts with the nuclei is performed using a number of techniques known in the art, including polyethylene glycol (see Pontecorvo “Polyethylene Glycol (PEG) in the Production of Mammalian Somatic Cell Hybrids” Cytogenet Cell Genet. 16 (1-5):399-400 (1976)), the direct injection of nuclei, sendai viral-mediated fusion, or other techniques known in the art. The cytoplasts and the nuclei are placed briefly in 1 mL of prewarmed 50% polyethylene glycol 1500 (Roche) for one minute. 20 mL of DMEM was then added over a five minute period to slowly remove the polyethylene glycol. The cells are centrifuged once at 130 g for five minutes and then taken back up in 50 μL of ES cell culture medium and placed beneath a feeder layer of fibroblasts under conditions to promote the outgrowth of an ES cell colony.

Another technique for performing step 2, also referred to as “cellular reconstitution” in the present method, is to fuse the remodeled nuclei with anucleate cytoplasmic blebs of germ-line cells, such as hES cells, attached to a physical substrate as is well known in the art (Wright & Hayflick, Exp. Cell Res. 96:113-121, (1975); & Wright & Hayflick, Proc. Natl. Acad. Sci., USA, 72:1812-1816, (1975). Briefly, the cytoplasmic volume of the germ-line cells is increased by adding 10 μM cytochalasin B for 20 hours prior to manipulation. The cells are then trypsinized and replated on sterile 18 mm coverslips, cylinders, or other physical substrate coated with material promoting attachment. The cells are plated at a density such that after an overnight incubation at 37° C. and one gentle wash with medium, the cells cover a portion, preferably about 90%, of the surface area of the coverslip or other substrate. The substrates are then placed in a centrifuge tube in a position such that centrifugation will result in the removal of the nuclei from the cytoplast containing 8 mL of 10% Ficoll-400 solution and centrifuged at 20,000 g at 36° C. for 60 minutes. Remodeled nuclei resulting from step one of the present method are then spread onto the coverslip or substrate with a density of at least that of the cytoplasts, preferable at least five times the density of the cytoplasts. Fusion of the cytoplasts with the nuclei is performed using polyethylene glycol (see Pontecorvo “Polyethylene Glycol (PEG) in the Production of Mammalian Somatic Cell Hybrids” Cytogenet Cell Genet. 16 (1-5)-399-400 (1976).

Briefly, in 1 mL of prewarmed 50% polyethylene glycol 1500 (Roche) in culture medium is placed over the coverslip or substrate for one minute. 20 mL of culture medium is then added drip-wise over a five minute period to slowly remove the polyethylene glycol. The entire media is then aspirated and replaced with culture medium. Techniques other than centrifugation such as vibration or physical removal of the nuclei using a micropipette may also be used.

In certain embodiments, the undifferentiated cells used in step 2 may first be manipulated to express or overexpress factors such as, for example, SOX2, NANOG, cMYC, OCT4, DNMT3B, any other factors listed in Table 7 and their non-human homologues, and/or other factors (e.g., regulatory RNA or constructs targeting the promoters of the genes listed in Table 7 and their non-human homologues) that confer undifferentiated cell behavior and facilitate reprogramming. Constructs encoding such factors may be transfected into undifferentiated cells, such as germ-line cells (e.g., blastomeres, morula cells, inner cell mass cells, ES cells, including hES cells, EG cells, or EC cells), by standard techniques known in the art. Examples of manipulating undifferentiated cells to express cellular factors are described above in Step 1. In alternative embodiments, such factors are introduced into the undifferentiated cells by injection or other methods. Examples of such methods to manipulate undifferentiated cells are likewise described above in Step 1.

In alternative embodiments, nuclear envelope reconstitution occurs following homologous recombination reactions that have modified target chromosomes. Thus, in one embodiment, as an optional step following nuclear envelope breakdown and chromatin condensation but before nuclear envelope reconstitution, DT40 extracts, or other recombination-proficient extracts or protein preparations, are added to the condensed chromosomes along with DNA targeting constructs such that recombination will result in the replacement of one or more genomic DNA sequences with the sequence (s) provided in the constructs. Exemplary embodiments of such methods are provided in Examples 16, 17, 18, and 19.

Step 3—Analysis of the Karyotype and Extent of Reprogramming

Cells reconstituted following steps 1 and 2 of the present method can be characterized to determine the pattern of gene expression and whether the reprogrammed cells display a pattern of gene expression similar to the expression pattern expected of undifferentiated cells such as ES cell lines using techniques well known in the art including transcriptomics (Klimanskaya et al., Cloning and Stem Cells, 6(3): 217-245 (2004)). Karyotypic analysis may be performed by means of chromosome spreads from mitotic cells, spectral karyotyping, assays of telomere length, total genomic hybridization, or other techniques well known in the art. In the case where the karyotype is normal, but telomere length or the extent of reprogramming is not complete, the cells may be used as nuclear donors and steps 1 and 2 repeated any number of times.

For example, the gene expression pattern of the reprogrammed cells may be compared to the gene expression pattern of embryonic stem cells or other undifferentiated cells. If the gene expression patterns are not similar, then the reprogrammed cell may be used in subsequent reprogramming steps until its gene expression is similar to the expression pattern of an undifferentiated cell (e.g., embryonic stem cell). The undifferentiated or embryonic stem cell to which the reprogrammed cell is compared may be from the same species as the donor differentiated somatic cell; alternatively, the undifferentiated or embryonic stem cell to which the reprogrammed cell is compared may be from the same species as the cytoplast or cytoplasmic bleb used in step 2. In some embodiments, a similarity in gene expression pattern exists between a reprogrammed cell and an undifferentiated cell (e.g., embryonic stem cell) if certain genes expressed in an undifferentiated cell are also expressed in the reprogrammed cell. For example, certain genes (e.g., telomerase) that are typically undetectable in differentiated somatic cells may be used to monitor the extent of reprogramming. Likewise, for certain genes, the absence of expression may be used to assess the extent of reprogramming. In certain embodiments, a cell may be considered reprogrammed if it expresses (1) E-cadherin (for human cells, CDH1; Accession No. NM_{—}004360.2) mRNA at levels of at least 5% of the expression level of the housekeeping gene GAPD (for human cells, NM_{—}002046.2) (data not shown); (2) detectable telomerase reverse transcriptase mRNA or exhibits telomerase activity as assessed by the TRAP assay (TRAPeze); and (2) LIN28 (NM_{—}024674.3; or its non-human equivalent for non-human cells) at levels of at least 5% of the housekeeping gene GAPD (for human cells, NM_{—}002046.2) (data not shown).

Other examples of the ways the different means of performing steps 1 and 2 of the present method can be combined include permeabilizing somatic cells by SLO, resealing the cells, and isolating the resulting partially-remodeled nuclei and then using the nuclei in the cellular reconstitution of step two. Also, the remodeled chromatin obtained by isolating differentiated cell nuclei, then exposing the nuclei to extracts from cells in the mitotic phase of the cell cycle to cause nuclear envelope breakdown and chromatin condensation, may then be transferred into the cytoplast of an ES cell, EC cell, or EG cell without reforming the nuclear envelope prior to cellular reconstitution. In addition, the somatic differentiated cell may be permeabilized as described above and exposed to extracts from oocytes or germ-line cells. The condensed chromatin from such cells may then be obtained, and then that chromatin may be fused with the recipient cytoplasts to yield reprogrammed cells. The fusion of chromatin with the cytoplasts is achieved by microinjection or is aided by fusigenic compounds as is known in the art (see, for example, U.S. Pat. Nos. 4,994,384 and 5,945,577). The fusigenic reagents include, but are not limited to, polyethylene glycol (PEG), lipophilic compounds such as Lipofectin™, Lipofectamin™, DOTAP™, DOSPA™, or DOPE™ For insertion of the chromatin into the cytoplasts, the coated chromatin is placed next to the cytoplast membrane and the complexes are maintained at a temperature of 20-30 degrees C. and monitored using a microscope. Once fusion has occurred, the medium is replaced with culture medium for the cultivation of undifferentiated cells and in culture conditions that promote the growth of said undifferentiated cells.

The cellular factors and methods of use listed herein may be used in alternative reprogramming techniques, such as in the methods disclosed by Collas and Robl, U.S. patent application Ser. No. 10/910,156, which is incorporated herein by reference in its entirety. The factors may, for example, be added to media (or alternatively expressed in cells used to obtain extract media) used to incubate a nucleus or chromatin mass from a donor cell under conditions that allow nuclear or cytoplasmic components from an undifferentiated cell to be added to the donor nucleus or chromatin mass.

The in vitro remodeling of somatic cell-derived DNA in step one of the present method is utilized as a model of reprogramming of a somatic cell and an assay useful in analyzing the molecular mechanisms of reprogramming. The selective addition, alteration, removal, or sequestration of particular molecular components, and the subsequent scoring of the extent of reprogramming or the extent of activation of telomerase and extension of telomere length allow the characterization of the role of particular molecules in the reprogramming that occurs during SCNT. The critical molecules characterized in this application of the present method are then used by their corresponding addition or deletion (e.g., by their addition if they facilitate reprogramming, or by their deletion if they inhibit reprogramming). Deletion can be achieved by, for instance, immune depletion, in oocytes or reprogramming extracts used in step one.

Specific molecular alterations can be introduced by—techniques well known in the art, including but not limited to, the addition of protein components, the removal of protein components such as by immunoprecipitation, the addition of other cellular components such as lipids, ions, DNA, or RNA. RNA may be prepared from oocytes, blastomeres, morula cells, ICM cells, ED cells or germ-line cells such as ES, EG, or EC cells. Total or fractions of the RNA such as microRNA are prepared as is well known in the art. This “germ-line RNA” is then introduced into the permeabilized cells of Example 14 at the point of incubating the cells at room temperature in order to allow the RNA to diffuse into the cells and improve the reprogramming of the somatic cells to an embryonic state once transplanted into the oocyte.

A common feature of the present method is that, regardless of which techniques are used to remodel the nuclear envelope and chromatin of a differentiated cell, at least two, and in some embodiments three, steps are used: one step wherein the chromatin and/or nuclear envelope are remodeled, a second step wherein the remodeled chromatin and/or nuclear envelope are reconstituted into a cytoplast to make a cell capable of cell division, and a third step wherein the resulting proliferating reprogrammed cells are analyzed to determine the degree of reprogramming and karyotype. If there is not a sufficient degree of reprogramming, the cells are cycled back to step one.

Somatic cells reprogrammed as described herein may be used to generate ES cells or ES cell lines including, but not limited to human ES cell lines. Since isolated human ES cells have a poor efficiency in generating cell lines, the reprogrammed cells of the present method may be aggregated together to facilitate the generation of stable ES cell lines. Such aggregation may include plating the cells at high density, placing the cells in a depression in the culture dish such that gravity brings the cells into close proximity, or the cells can be co-cultured with feeder cells or with existing ES cell lines.

Human embryonic cells, e.g., human ES cells may be cultured on feeder cells, e.g., mouse embryonic fibroblasts, or human feeder cells such as fibroblasts (e.g., human foreskin fibroblasts, human skin fibroblasts, human endometrial fibroblasts, human oviductal fibroblasts) and placental cells. In one embodiment, the human feeder cells may be autologous feeder cells derived from the same culture of reprogrammed cells by direct differentiation and the clonal isolation of cells useful in ES cell derivation. The human embryonic cells are grown in ES cell medium or any medium that supports growth of the embryonic cells, e.g., Knockout DMEM (Invitrogen Cat #10829-018).

Alternatively, the reprogrammed cells obtained from the methods of the present method may be co-cultured in juxtaposition with exiting ES cell lines. Exemplary human embryonic cells include, but are not limited to, embryonic stem cells, such as from already established lines, embryo carcinoma cells, murine embryonic fibroblasts, other embryo-like cells, cells of embryonic origin or cells derived from embryos, many of which are known in the art and available from the American Type Culture Collection, Manassas, Va. 20110-2209, USA, and other sources.

The embryonic cells may be added directly to the cultured reprogrammed cells or may be grown in close proximity to, but not in direct contact with, the cultured reprogrammed cells. The method comprises the step of directly or indirectly contacting the cultured reprogrammed cells with embryonic cells. Alternatively, the culture of reprogrammed cells and the culture of embryonic cells are indirectly connected or merged. This can be achieved by any method known in the art including, for example, using light mineral oil such as Cooper Surgical ACT# ART4008, paraffin oil or Squibb's oil. The connections can be made by using a glass capillary or similar device. Such indirect connections between the cultured reprogrammed cells and the embryonic cells allows gradual mixing of the embryo medium (in which the reprogrammed cells are cultured) and the ES cell medium (in which the human embryonic cells are grown).

In another embodiment, the reprogrammed cells may be co-cultured with a human embryo. For example, the reprogrammed cells are co-cultured with the embryo in a microdroplet culture system or other culture system, known in the art, but which does not permit cell-cell contact but could permit cell-secreted factor and/or cell-matrix contact. The volume of the microdrop may be reduced, e.g., from 50 microliters to about 5 microliters to intensify the signal. In another embodiment the embryonic cells may be from a species other than human, e.g., non-human primate or mouse.

After about 3-4 days, the reprogrammed cells exhibit properties of ES cells. While not wishing to be bound by any particular theory, it is believed that over a period of days or weeks the cultured reprogrammed cells exhibit facilitated ES cell growth perhaps as a result of factors secreted by the human embryonic cells or by the extracellular matrix. The above-described methods for producing ES cells are described in application PCT/US05/39776, U.S. Ser. No. 11/267,555 and 60/831,698, which are incorporated herein in their entirety. Properties of ES cells or an ES cell line may include, without limitation, the expression of telomerase and/or telomerase activity, and the expression of one or more known ES cell markers.

In certain embodiments, the reprogrammed cell culture conditions may include contacting the cells with factors that can inhibit or otherwise potentiate the differentiation of the cells, e.g., prevent the differentiation of the cells into non-ES cells, trophectoderm or other cell types. Such conditions can include contacting the cultured cells with heparin or introducing reprogramming factors into the cells or extracts as described herein. In yet another embodiment, expression of cdx-2 is prevented by any means known in the art including, without limitation, introducing CDX-2 RNAi into the reprogrammed cells, thereby inhibiting differentiation of the reprogrammed cells into TS cells, thereby insuring that said cells could not lead to a competent embryo.

In another embodiment, the reprogrammed cells resulting from steps 1 and 2 of the methods are directly used to produce differentiated progeny without the production of an ES cell line. Thus, in one aspect, the present method provides a disclosure for producing differentiated progenitor cells, comprising:

(i) obtaining reprogrammed cells using steps 1-2 or 1-3 of the methods of this disclosure; and (ii) inducing differentiation of the reprogrammed cells to produce differentiated progenitor cells without producing an embryonic stem cell line. The differentiated progenitor cells can be used to derive cells, tissues and/or organs which are advantageously used in the area of cell, tissue, and/or organ transplantation which include all of the cells and applications described herein for ES-derived cells and tissues.

In the past, long-term culture of inner cell mass cells was used to produce embryonic stem cell lines. Subsequently, the embryonic stem cells were cultured and conditionally genetically-modified, and induced to differentiate in order to produce cells to make cells for therapy. Co-owned pending U.S. pending application 2005/0265976A1 describes a method of producing differentiated progenitor cells from inner cell mass cells or morula-derived cells by directly inducing the differentiation of those cells without producing an embryonic stem cell line. The application also describes the use of said differentiated cells, tissues, and organs in transplantation therapy. In the method of the present disclosure, reprogrammed cells derived from steps 1-2 or 1-3 as described herein are induced to directly differentiate into differentiated progenitor cells which are then used for cell therapy and for the generation of cells, tissues, and organs for transplantation. If desired, genetic modifications can be introduced, for example, into somatic cells prior to reprogramming or into the chromatin in the extracts as described herein. Thus, the differentiated progenitor cells of the present method do not possess the pluripotency of an embryonic stem cell, or an embryonic germ cell, and are, in essence, tissue-specific partially or fully differentiated cells. These differentiated progenitor cells may give rise to cells from any of three embryonic germ layers, i.e., endoderm, mesoderm, and ectoderm. For example, the differentiated progenitor cells may differentiate into bone, cartilage, smooth muscle, dermis with a prenatal pattern of gene expression and capable of promoting scarless wound repair, and hematopoietic or hemangioblast cells (mesoderm); definitive endoderm, liver, primitive gut, pancreatic beta cells, progenitors of pancreatic beta cells, and respiratory epithelium (endoderm); or neurons, glial cells, hair follicles, or eye cells including retinal neurons and retinal pigment epithelium using techniques known in the art, or using techniques described in the pending applications PCT/US2006/013573 filed Apr. 11, 2006, and U.S. Application No. 60/811,908, filed Jun. 11, 2006, which are both incorporated in their entirety by reference.

One advantage of these methods is that the cells obtained by steps 1-2 or steps 1-3 can be differentiated without prior purification or establishment of a cell line. The cells obtained by the methods disclosed herein can be differentiated without the selection or purification of the cells. Accordingly in certain embodiments, a heterogeneous population of cells comprising reprogrammed cells are differentiated into a desired cell type. In one embodiment, a mixture cells obtained from steps 1-2 as described herein are exposed to one or more differentiation factors and cultured in vitro. Thus in certain embodiments, there is no need to purify the reprogrammed cells or to establish an ES or other cell line before differentiation. In one embodiment, a heterogeneous population of cells comprising reprogrammed cells is permeabilized to facilitate access to differentiation factors and subsequent differentiation.

Furthermore, it is not necessary for the differentiated progenitor cells of the present method to express the catalytic component of telomerase (TERT) and be immortal, or that the progenitor cells express cell surface markers found on embryonic stem cells such as the cell surface markers characteristic of primate embryonic stem cells: positive for SSEA-3, SSEA-4, TRA-1-60, TRA-1-81, alkaline phosphatase activity, and negative for SSEA-1. Moreover, the differentiated progenitor cells of the present method may be distinct from embryoid bodies, i.e., embryoid bodies are derived from embryonic stem cells whereas the differentiated stem cells of the present method may be derived from reprogrammed cells without the production of ES cell lines.

Applications

The cells resulting from steps 1 and 2 of the methods of this method are plated in conditions that promote the growth of ES cells, such as hES cells, as is well known in the art. Briefly, the cells may be left on the substrate in which the enucleated cytoplasts are prepared, or they may be trypsinized and centrifuged at 700×g for 3 minutes and taken up into a sterile Pasteur pipette and placed under a feeder monolayer to concentrate and to co-localize the cells. The cells may be co-cultured with other vigorously-growing ES cell lines that can be easily removed by means such as suicide induction after encouraging the growth of the reprogrammed stem cells. The reprogrammed cells may also be concentrated into a small surface area of the growth surface by plating in a small cloning cylinder as well as be cultured by other techniques well known in the art.

In another aspect, the method comprises the utilization of cells derived from the reprogrammed cells of the present method in research and in therapy. Such reprogrammed pluripotent or totipotent cells may be differentiated into any of the cells in the body including, without limitation, skin, cartilage, bone skeletal muscle, cardiac muscle, renal, hepatic, blood and blood forming, vascular precursor and vascular endothelial, pancreatic beta, neurons, glia, retinal, inner ear follicle, intestinal, lung, cells.

In a particular embodiment, the reprogrammed cells may be differentiated into cells with a dermatological prenatal pattern of gene expression that is highly elastogenic or capable of regeneration without causing scar formation. Dermal fibroblasts of mammalian fetal skin, especially corresponding to areas where the integument benefits from a high level of elasticity, such as in regions surrounding the joints, are responsible for synthesizing de novo the intricate architecture of elastic fibrils that function for many years without turnover. In addition, early embryonic skin is capable of regenerating without scar formation. Cells from this point in embryonic development made from the reprogrammed cells of the present method are useful in promoting scarless regeneration of the skin including forming normal elastin architecture. This is particularly useful in treating the symptoms of the course of normal human aging, or in actinic skin damage, where there can be a profound elastolysis of the skin resulting in an aged appearance including sagging and wrinkling of the skin.

In another embodiment, the reprogrammed cells are exposed to inducers of differentiation to yield other therapeutically-useful cells such as retinal pigment epithelium, definitive endoderm, pancreatic beta cells and precursors to pancreatic beta cells, hematopoietic precursors and hemangioblastic progenitors, neurons, respiratory cells, muscle progenitors, cartilage and bone-forming cells, cells of the inner ear, neural crest cells and their derivatives, gastrointestinal cells, liver cells, kidney cells, smooth and cardiac muscle cells, dermal progenitors including those with a prenatal pattern of gene expression useful in promoting scarless wound repair, as well as many other useful cell types of the endoderm, mesoderm, and endoderm. Such inducers include but are not limited to: cytokines such as interleukin-alpha A, interferon-alpha A/D, interferon-beta, interferon-gamma, interferon-gamma-inducible protein-10, interleukin-1-17, keratinocyte growth factor, leptin, leukemia inhibitory factor, macrophage colony-stimulating factor, and macrophage inflammatory protein-1 alpha, 1-beta, 2, 3 alpha, 3 beta, and monocyte chemotactic protein 1-3, 6Ckine, activin A, amphiregulin, angiogenin, B-endothelial cell growth factor, beta cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropoietin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocyte macrophage colony stimulating factor, GRO-alpha/MGSA, GRO-beta, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, insulin-like growth factor II, nerve growth factor, neurotophin-3, 4, oncostatin M, placenta growth factor, pleiotrophin, rantes, stem cell factor, stromal cell-derived factor 1B, thrombopoietin, transforming growth factor-(alpha, beta 1, 2, 3, 4, 5), tumor necrosis factor (alpha and beta), vascular endothelial growth factors, and bone morphogenic proteins, enzymes that alter the expression of hormones and hormone antagonists such as 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine/leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopressin, extracellular matrix components such as fibronectin, proteolytic fragments of fibronectin, laminin, tenascin, thrombospondin, and proteoglycans such as aggrecan, heparan sulphate proteoglycan, chondroitin sulphate proteoglycan, and syndecan. Other inducers include cells or components derived from cells from defined tissues used to provide inductive signals to the differentiating cells derived from the reprogrammed cells of the present method. Such inducer cells may derive from human, nonhuman mammal, or avian, such as specific pathogen-free (SPF) embryonic or adult cells.

Differentiated progeny may also be derived from reprogrammed ES cell lines or directly differentiated from reprogrammed cells using clonal isolation procedures as described in the pending application PCT/US2006/013573 filed Apr. 11, 2006, and U.S. Application No. 60/811,908, filed Jun. 7, 2006, which are incorporated herein by reference. Methods of differentiating reprogrammed cells obtained by the methods disclosed herein may comprise a step of permeabilization of the reprogrammed cell. For example, ES cell lines generated by the reprogramming techniques described herein, or alternatively a heterogeneous mixture of cells comprising reprogrammed cells, may be permeabilized before exposure to one or more differentiation factors or cell extract or other preparation comprising differentiation factors. Permeabilization techniques include, for example, incubation of cell(s) with a detergent, such as digitonin, or a bacterial toxin, such as Streptolysin O, or by methods as described in PCT/US2006/013573 filed Apr. 11, 2006, and U.S. Application No. 60/811,908, filed Jun. 7, 2006, which are incorporated my means of reference. In certain embodiments, reprogrammed cells are permeabilized and then exposed to extract from beta cells (e.g., bovine beta cells).

These methods also enable the generation of cell lines homozygous or hemizygous for MHC antigens. Hemizygous or homozygous HLA cell lines may be generated in differentiated cell lines that are dedifferentiated to generate a totipotent or pluripotent stem cell line that is homozygous at the HLA locus. See for example U.S. Patent Publication No. US 2004/0091936, filed May 14, 2004, the disclosure of which is incorporated by reference herein. For instance, differentiated cells can be dedifferentiated using the reprogramming methods disclosed herein to generate a totipotent or pluripotent stem cell. Totipotent and pluripotent stem cells homozygous for histocompatibility antigens, e.g., MHC antigens, can be produced by remodeling the nucleus of a somatic cell homozygous for the antigens and then reconstituting the remodeled nucleus as described in the present disclosure. Cytoplasm from an undifferentiated cell may be added to isolated nuclei or chromatin from differentiated cells, or differentiated cells that are permeabilized. Following reprogramming of the somatic cell, the resulting dedifferentiated, pluripotent, stem or stem-like homozygous cell may be differentiated into a desired cell type. Methods for inducing re-differentiation into a cell type other than that of the initial differentiated cells are described, for example, in co-owned and co-pending U.S. publication 20030027330, filed Apr. 2, 2002, the disclosure of which is incorporated herein by reference in its entirety. Further, during step 1 of de-differentiation, the nucleus remodeled in step one may be modified by homologous recombination. The addition of extracts from cells such as DT40 known to have a high level of homologous recombination along with DNA targeting constructs will then yield cells after reconstitution in step 2 and screening in step 3 that have a desired genetic modification and that are homozygous for MHC antigen.

Many of the steps in the present method are time intensive and require skilled technicians to perform the steps at a high level of quality. To decrease cost and increase quality and reproducibility, many of the steps described above can be automated through the use of robotics. Robotic platforms can, for example, culture cells, introduce buffers and other reagents, thaw and introduce extracts, and reconstitute cells in step 2.

The present method is commercialized by regional centers that receive differentiated cells from animals or humans in need of cell therapy and perform steps 1-2 or 1-3 of these methods, and return either the reprogrammed pluripotent stem cells to a clinical center where they are differentiated into a therapeutically-useful cell type, or the differentiation is performed in the regional center and the cells ready for transplantation are shipped in live cultures or in a cryopreserved state to the health care provider.

### DEFINITIONS

“iPS cell” (induced pluripotent cell)—In the present disclosure this refers to a pluripotent cell that has been created by transforming a somatic cell through contact with reprogramming agents, e.g., using viral transduction to cause the somatic cell to express of one or more reprogramming polypeptides.

“Genetically intact iPS cell”—In the present disclosure this refers to an iPS cell that has been made without the introduction of undesired genetic modifications. For example, a genetically intact iPS cell may be made using recombinant reprogramming polypeptides and/or reprogramming agents comprised in a donor cell cytoplasm. A genetically intact iPS cell optionally includes one or more desired genetic modifications.

The term “protein transduction domain” (“PTD”) refers to any amino acid sequence that translocates across a cell membrane into cells or confers or increases the rate of, for example, another molecule (such as, for example, a protein domain) to which the PTD is attached, to translocate across a cell membrane into cells. The protein transduction domain may be a domain or sequence that occurs naturally as part of a larger protein (e.g., a PTD of a viral protein such as HIV TAT) or may be a synthetic or artificial amino acid sequence.

“Dedifferentiation”—In the present disclosure, dedifferentiation refers to reversing the differentiated state of a cell to an embryonic or progenitor state. An example of dedifferentiation is the changes in a differentiated cell, e.g., human somatic cell in tissue culture, that result upon introduction of cytoplasm from a more primitive, less differentiated cell type, e.g., an oocyte or other embryonic cell. (also referred to as ‘dedifferentiation’), and these early stage cells could then be differentiated to a desired cell type.

“Transdifferentiation”—In the present disclosure, transdifferentiation refers to conversion of one differentiated cell type to another desired differentiated cell type. An example of transdifferentiation is the changes in a differentiated cell, e.g., human somatic cell in tissue culture, that result upon introduction of cytoplasm from a cell of a different differentiated cell type than the recipient cell.

“Ooctye”—In the present disclosure, this refers to any oocyte, preferably a mammalian oocyte, that develops from an oogonium and, following meiosis, becomes a mature ovum.

“Metaphase II ooctye”—The preferred stage of maturation of oocytes used for nuclear transfer (First and Prather, Differentiation, 48:1-8). At this stage, the oocyte is sufficiently “prepared” to treat an introduced donor cell or nucleus as it does a fertilizing sperm.

“Donor Cell”—In the present disclosure, this refers to a cell wherein some or all of its cytoplasm is transferred to another cell (“recipient cell”). The donor cell is typically a primitive or embryonic cell type, such as an oocyte, blastomere, inner cell mass cell, teratocarcinoma cells, spermatogonia, mature frog, etc. or another cell type that is in a less differentiated state or more primitive state or a different cell type than the recipient cell. In general, it is preferred that the donor cytoplasm be obtained from oocytes or other embryonic cells that are in an undifferentiated or substantially undifferentiated state.

“Recipient Cell”—This refers to a cell into which a reprogramming agent is introduced. The recipient cell can be any differentiated cell type. Suitable examples thereof include epithelial cells, endothelial cells, fibroblasts, keratinocytes, melanocytes and other skin cell types, muscle cells, bone cells, immune cells such as T and B-lymphocytes, oligodendrocytes, dendritic cells, erythrocytes and other blood cells; pancreatic cells, neural and nerve cell types, stomach, intestinal, esophageal, lung, liver, spleen, kidney, bladder, cardiac, thymus, corneal, and other ocular cell types, etc. In general, the methods have application in any application wherein a source of cells that are in a less differentiated state would be desirable.

“Reprogramming” herein broadly encompasses the conversion of a cell or cell nucleus into a less differentiated cell (dedifferentiated cell) preferably into a totipotent or pluripotent cell or it alternatively refers to conversion of the cell or cell nucleus into a cell of a different cell lineage or cell type. In the present disclosure this is preferably effected using one or more reprogramming factors which may comprise endogenous reprogramming factors or fusions containing which in addition may comprise one or more NLS or PTD sequences to facilitate cell internalization ad nuclear internalization.

“Reprogramming agent”—Exemplary reprogramming agents include polypeptides, small molecules, nucleic acids, etc. Exemplary reprogramming agents include Oct4, Sox2, Nanog, Klf4, c-Myc, and Lin28, and the genes listed in Tables 1 and 2 and homologs or functional fragments or variants thereof, which may be in the form of polypeptides and/or nucleic acids that encode these polypeptides, and may be comprised in a cell extract. For example, a reprogramming agent may be comprised in an extract from a cell that expresses a reprogramming agent naturally or has been induced to express a reprogramming agent. Reprogramming agents also include agents that inhibit gene expression, e.g., siRNA targeting genes whose knock-down promotes reprogramming. Reprogramming may be conducted using a defined set of agents, such as one or more recombinant fusion proteins, or a cell extract which is optionally fractionated to enrich the reprogramming agent(s) contained therein, or a mixture of a cell extract and a defined agent (e.g. made by adding a defined agent to a cell extract or by engineering the cell from which the extract is made to cause it to generate the defined agent). For example, reprogramming may be effected by transfer of all or part of the cytoplasm of a donor cell, wherein such donor cell is of a more primitive cell type or a different cell type relative to the recipient cell.

“Blastomere”—Embryonic, substantially undifferentiated cells contained in blastocyst stage embryos.

“Embryonic cell or embryonic cell type”—In the present disclosure, this will refer to any cell, e.g., oocyte, blastomere, embryonic stem cell, inner cell mass cell, or primordial germ cell, wherein the introduction of cytoplasm therefrom into a differentiated cell, e.g., human somatic cell in tissue culture, results in dedifferentiation and/or lengthening of the life-span of such differentiated cell.

“Cell having altered life-span”—In the present disclosure this refers to the change in cell life-span (lengthening) that results when cytoplasm of a more primitive or less differentiated cell type, e.g., an embryonic cell or embryonic cell type, e.g., oocyte or blastomere, is introduced into a desired differentiated cell, e.g., a cultured human somatic cell.

“Embryonic stem cell (ES cell)”—In the present disclosure this refers to an undifferentiated cell that has the potential to develop into an entire organism, i.e., a cell that is able to propagate indefinitely, maintaining its undifferentiated state and, when induced to differentiate, be capable of giving rise to any cell type of the body. ES cells, the progeny of the inner cell mass (ICM) of a blastocyst, remain pluripotent, maintain normal karyotype through multiple passages in culture, and can differentiate into derivatives of all three germ layers in vitro and in vivo, and can make teratomas in laboratory animals.

“Nuclear Transfer”—Introduction of cell or nuclear DNA of donor cell into enucleated oocyte which cell or nucleus and oocyte are then fused to produce a nuclear transfer fusion or nucleus fusion embryo. This NT fusion may be used to produce a cloned embryo or offspring or to produce ES cells.

“Telomerase”—A ribonucleoprotein (RNP) particle and polymerase that uses a portion of its internal RNA moiety as a template for telomere repeat DNA synthesis (U.S. Pat. No. 5,583,016; Yu et al, Nature, 344:126 (1990); Singer and Gottschling, Science, 266:404 (1004); Autexier and Greider, Genes Develop., 8:563 (1994); Gilley et al, Genes Develop., 9:2214 (1995); McEachern and Blackburn, Nature, 367:403 (1995); Blackburn, Ann. Rev. Biochem., 61:113 (1992); Greider, Ann Rev. Biochem., 65:337 (1996).) The activity of this enzyme depends upon both its RNA and protein components to circumvent the problems presented by end replication by using RNA (i.e., as opposed to DNA) to template the synthesis of telomeric DNA. Telomerases extend the G strand of telomeric DNA. A combination of factors, including telomerase processivity, frequency of action at individual telomeres, and the rate of degradation of telomeric DNA, contribute to the size of the telomeres (i.e., whether they are lengthened, shortened, or maintained at a certain size). In vitro telomerases may be extremely processive, with the Tetrahymena telomerase adding an average of approximately 500 bases to the G strand primer before dissociation of the enzyme (Greider, Mol. Cell. Biol., 114572 (1991).) WO 98/14593, published Apr. 9, 1998, by Cech et al, reports telomerase nucleic acid sequences derived from Eeuplotes aediculatus, Saccharomyces, Schizosaccharomyces, and human, as well as polypeptides comprising telomerase protein subunits. Also, WO 98/14592, to Cech et al, published Apr. 9, 1998, discloses compositions containing human telomerase reverse transcriptase, the catalytic protein subunit of human telomerase. Also, U.S. Pat. Nos. 5,837,857 and 5,583,414 describe nucleic acids encoding mammalian telomerases. Still further, U.S. Pat. No. 5,830,644, issued to West et al; U.S. Pat. No. 5,834,193, issued to Kzolowski et al, and U.S. Pat. No. 5,837,453, issued to Harley et al, describe assays for measuring telomerase length and telomerase activity and agents that affect telomerase activity.

“Genetically modified or altered”—In the present disclosure this refers to cells that contain one or more modifications in their genomic DNA, e.g., additions, substitutions and/or deletions.

“Totipotent”—In the present disclosure this refers to a cell that gives rise to all of the cells in a developing body, such as an embryo, fetus, an animal. The term “totipotent” can also refer to a cell that gives rise to all of the cells in an animal. A totipotent cell can give rise to all of the cells of a developing cell mass when it is utilized in a procedure for creating an embryo from one or more nuclear transfer steps. An animal may be an animal that functions ex utero. An animal can exist, for example, as a live born animal. Totipotent cells may also be used to generate incomplete animals such as those useful for organ harvesting, e.g., having genetic modifications to eliminate growth of a head such as by manipulation of a homeotic gene.

“Ungulate”—In the present disclosure this refers to a four-legged animal having hooves. In other preferred embodiments, the ungulate is selected from the group consisting of domestic or wild representatives of bovids, ovids, cervids, suids, equids, and camelids. Examples of such representatives are cows or bulls, bison, buffalo, sheep, big-horn sheep, horses, ponies, donkeys, mule, deer, elk, caribou, goat, water buffalo, camels, llama, alpaca, and pigs. Especially preferred in the bovine species are Bos Taurus, Bos Indicus, and Bos buffaloes cows or bulls.

“Immortalized” or “permanent” cell—These terms as used in the present disclosure in reference to cells can refer to cells that have exceeded the Hayflick limit. The Hayflick limit can be defined as the number of cell divisions that occur before a cell line becomes senescent. Hayflick set this limit to approximately 60 divisions for most non-immortalized cells. See, e.g., Hayflick and Moorhead, 1971, Exp. Cell. Res., 25:585-621; and Hayflick, 1965, Exp. Cell Research, 37:614-636, incorporated herein by reference in their entireties, including all figures, tables and drawings. Therefore, an immortalized cell line can be distinguished from non-immortalized cell lines if the cells in the cell line are able to undergo more than 60 divisions. If the cells of a cell line are able to undergo more than 60 cell divisions, the cell line is an immortalized or permanent cell line. The immortalized cells of the present disclosure are preferably able to undergo more than 70 divisions, are more preferably able to undergo more than 90 divisions, and are most preferably able to undergo more than 90 cell divisions. Typically, immortalized or permanent cells can be distinguished from non-immortalized and non-permanent cells on the basis that immortalized and permanent cells can be passaged at densities lower than those of non-immortalized cells. Specifically, immortalized cells can be grown to confluence (e.g., when a cell monolayer spreads across an entire plate) when plating conditions do not allow physical contact between the cells. Hence, immortalized cells can be distinguished from non-immortalized cells when cells are plated at cell densities where the cells do not physically contact one another.

“Culture”—In the present disclosure this term refers to one or more cells that are static or undergoing cell division in a liquid medium. Nearly any type of cell can be placed in cell culture conditions. Cells may be cultured in suspension and/or in monolayers with one or more substantially similar cells. Cells may be cultured in suspension and/or in monolayers with heterogeneous population cells. The term heterogeneous as utilized in the previous sentence can relate to any cell characteristics, such as cell type and cell cycle stage, for example. Cells may be cultured in suspension and/or in monolayers with feeder cells.

“Feeder Cells”—This refers to cells grown in co-culture with other cells. Feeder cells include, e.g., fibroblasts, fetal cells, oviductal cells, and may provide a source of peptides, polypeptides, electrical signals, organic molecules (e.g., steroids), nucleic acid molecules, growth factors, cytokines, and metabolic nutrients to cells co-cultured therewith. Some cells require feeder cells to be grown in tissue culture.

“Embryo”—In the present disclosure this refers to a developing cell mass that has not implanted into the uterine membrane of a maternal host. Hence, the term “embryo” as used herein can refer to a fertilized oocyte, a cybrid (defined herein), a pre-blastocyst stage developing cell mass, and/or any other developing cell mass that is at a stage of development prior to implantation into the uterine membrane of a maternal host. Embryos of the present disclosure may not display a genital ridge. Hence, an “embryonic cell” is isolated from and/or has arisen from an embryo.

“Fetus”—In the present disclosure refers to a developing cell mass that has implanted into the uterine membrane of a maternal host. A fetus can include such defining features as a genital ridge, for example. A genital ridge is a feature easily identified by a person of ordinary skill in the art and is a recognizable feature in fetuses of most animal species.

“Fetal cell”—as used herein can refer to any cell isolated from and/or has arisen from a fetus or derived from a fetus.

“Non-fetal cell”—refers to a cell that is not derived or isolated from a fetus.

“Senescence”—In the present disclosure this refers to the characteristic slowing of growth of non-immortal somatic cells in tissue culture after cells have been maintained in culture for a prolonged period. Non-immortal cells characteristically have a defined life-span before they become senescent and die. The present disclosure alleviates or prevents senescence by the introduction of cytoplasm from a donor cell, typically an oocyte or blastomere, into a recipient cell, e.g., a cultured human somatic cell.

The term “cellular reconstitution” refers to the transfer of a nucleus or chromatin to cellular cytoplasm so as to obtain a functional cell.

The term “chromatin transfer” (CT) refers to the cellular reconstitution of condensed chromatin.

The term “condensed chromatin” refers to DNA not enclosed by a nuclear envelope. Condensed chromatin my result, for example, by exposing a nucleus to a mitotic extract such as from an M1 or an Mil oocyte or other mitotic cell extract, by transferring a nucleus into an M1 or an MII oocyte or other mitotic cell and retrieving the resulting condensed chromatin following the breakdown of the nuclear envelope. Condensed chromatin refers to chromosomes that are in a greater degree of compaction than the degree of compaction that occurs in any phase of the cell cycle other than metaphase.

The term “cytoplasmic bleb” refers to the cytoplasm of a cell bound by an intact, or permeabilized, but otherwise intact plasma membrane but lacking a nucleus. It is used interchangeably and synonymously with the term “enucleate cytoplast” and “enucleated cytoplasm”, unless the term “enucleate cytoplasm” is explicitly used in the context of an extract in which the plasma membrane has been removed.

The term “cytoplasmic transfer” (CyT) refers to any number of techniques known in the art for juxtaposing the nucleus of a somatic cell with the cytoplasm of an undifferentiated cell. Such techniques include, but are not limited to, the direct transfer of said undifferentiated cytoplasm into the cytoplasm of a differentiated cell, the permeabilization of a somatic cell to allow the diffusion of undifferentiated cell cytoplasm into the somatic cell, or the transfer of the somatic cell nucleus into a cytoplasmic bleb of an undifferentiated cell.

The term “differentiated cell” refers to any cell from any vertebrate species in the process of differentiating into a somatic cell lineage or having terminally differentiated into the type of cell it will be in the adult organism.

The term “pluripotent stem cells” refers to animal cells capable of differentiating into more than one differentiated cell type. Such cells include ES cells, EG cells, EDCs, ED-like cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “embryonic stem cells” (ES cells) refers to, for example, cells derived from the inner cell mass of blastocysts or morulae that have been serially passaged as cell lines. The ES cells may be derived from fertilization of an egg cell with sperm or DNA, nuclear transfer, parthenogenesis or by means to generate ES cells with homozygosity in the WIC region. hES cells are human ES Cells.

The term “fusigenic compound” refers to a compound that increases the likelihood that a condensed chromatin or nucleus is fused with and incorporated into a recipient cytoplasmic bleb resulting in a viable cell capable of subsequent cell division. Such fusigenic compounds may, by way of nonlimiting example, increase the affinity of a condensed chromatin or a nucleus with the plasma membrane. Alternatively, the fusigenic compound may increase the likelihood of the joining of the lipid bilayer of the target cytoplasmic bleb with the condensed chromatin, nuclear envelope of an isolated nucleus, or the plasma membrane of a donor cell.

The term “heteroplasmon” refers to a cell resulting from the fusion of a cell containing a nucleus and cytoplasm with the cytoplast of another cell.

The term “human embryo-derived cells” (hEDC) refer to blastomeres, morula-derived cells, blastocyst-derived cells including those of the inner cell mass, embryonic shield, or epiblast, or other totipotent or pluripotent stem cells of the early embryo, including primitive endodertn, ectoderm, and mesoderm and their derivatives, but excluding hES cells that have been passaged as cell lines. The hEDC cells may be derived from fertilization of an egg cell with sperm or DNA, nuclear transfer, parthenogenesis, or by means to generate hES cells with homozygosity in the HIA region.

The term “human embryo-derived-like cells” (hED-like) refer to pluripotent stem cells produced by the present invention that are not cultured so as to retain the characteristics of ES cells, but like morula-derived cells, blastocyst-derived cells including those of the inner cell mass, embryonic shield, or epiblast, or other totipotent or pluripotent stem cells of the early embryo, including primitive endoderm, ectoderm, and mesoderm and their derivatives that have not been cultured so as to maintain stable hES lines, are capable of differentiating into any of the somatic cell differentiated types. The hED-like cells may be derived with genetic modifications, including modified so as to lack genes of the MHC region, to be hemizygous or homozygous in this region.

The term “nuclear remodeling” refers to the artificial alteration of the molecular composition of the nuclear lamina or the chromatin of a cell.

The term “permeabilization” refers to the modification of the plasma membrane of a cell such that there is a formation of pores enlarged or generated in it or a partial or complete removal of the plasma membrane.

The term “pluripotent” refers to the characteristic of a stem cell that said stem cell is capable of differentiating into a multitude of differentiated cell types.

The term “undifferentiated cell” refers to an oocyte, an undifferentiated cell such as an ES, EG, ICM, ED, EC, teratocarcinonaa cell, blastomere, morula, or germ-line cell.

Throughout this specification and claims, the word “comprise,” or variations such as “comprises” or “comprising”, will be understood to imply the inclusion of a stated integer or group of integers but not the exclusion of any other integer or group of integers.

Further, unless otherwise required by context, singular terms shall include pluralities and plural terms shall include the singular.

Unless otherwise defined, all technical and scientific terms used herein have the same meaning as commonly understood by one of ordinary skill in the art. In case of conflict, the present specification, including definitions, will control.

Abbreviations

3-D—three dimensional

BFFs—bovine fibroblasts

bFGF—basal fibroblast growth factor

BMP-2—bone morphogenic protein-2

CalR1—glial marker

CB—cytochalasin B

CD14—lipopolysacharide receptor

CD34—leukocyte common antigen

CD45—blood cell marker

CNF—necrosis factor

CNS—central nervous system

CNTF—cilia neurotropic factor

CT—Chromatin Transfer

CyT—Cytoplasmic Transfer

BSA—bovine serum albumin

ECM—extracellular matrix

ESC—embryonic stem cells

FCS—fetal calf serum

GFs—growth factors

DMAP—Dimethylaminopurine

DMEM—Dulbecco's modified minimum essential medium

DMSO—dimethylsulfoxide

EGF—epidermal growth factor

En-1—enolase

FGFR3—fibroblast growth factor receptor 3

G1/G0—gap phases of the cell cycle

GABA—gamma-amino butyric acid

GFAP—glial fibrilarin associated protein

HPLC—high pressure liquid chromatography

ICC—immunocytochemistry

ICM—inner cell mass

IgG—immunoglobulin G

Nurr-1—nuclear receptor

Pax8—neuronal inducer

PDGF—platelet derived growth factor

PERVS—porcine endogenous retroviruses

RT-PCR—reverse transcription-polymerase chain reaction

SCID—severe combined immunodeficiency

SHH—sonic hedgehog

T3—tyroxin

TH—tyrosine hydroxylase

TUJ1—glial marker

EC Cells—Embryonal Carcinoma Cells

ED Cells—Embryo-derived cells are cells derived from a zygote, blastomeres, morula or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane of an oocyte or blastomere to produce a cell line. The resulting cell line may be either a differentiated cell line or the cells may be maintained as undifferentiated ES cells. Therefore ED cells are inclusive of ES cells and cells derived by directly differentiating cells from a mammalian preimplantation embryo. hED Cells are human embryo-derived cells derived from, for example, human preimplantation embryos. Human embryo-derived cells may refer to morula-derived cells, blastocyst-derived cells including those of the inner cell mass, embryonic shield, or epiblast, or other totipotent or pluripotent stem cells of the early embryo, including primitive endoderm, ectoderm, and mesoderm and their derivatives, but excluding hES cells that have been passaged as cell lines. The hED cells may be derived from fertilization of an egg cell with sperm or DNA, nuclear transfer, parthenogenesis, or by means to generate hES cells with homozygosity in the HLA region.

ES Cell—Embryonic stem cells derived from a zygote, blastomeres, morula or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce a cell. hES Cells are human embryonic stem cells, derived from, for example, human preimplantation embryos. hES Cells may be derived from the inner cell mass of human blastocysts or morulae that have been serially passaged as cell lines. The hES cells may be derived from fertilization of an egg cell with sperm or DNA, nuclear transfer, parthenogenesis, or by means to generate hES cells with homozygosity in the HLA region.

GCL—Germ cell-less

HSE—Human skin equivalents are mixtures of cells and biological or synthetic matrices manufactured for testing purposes or for therapeutic application in promoting wound repair.

INM—Inner nuclear membrane

MBS—Magnesium buffered saline

MiRNA—Micro RNA

NPC—Nuclear Pore Complex

NT—Nuclear Transfer

ONM—Outer nuclear membrane

PEG—Polyethylene glycol

PS fibroblasts—Pre-scarring fibroblasts are fibroblasts derived from the skin of early gestational skin or derived from ED cells that display a prenatal pattern of gene expression with that they promote the rapid healing of dermal wounds without scar formation.

SCNT—Somatic Cell Nuclear Transfer

SLO—Streptolysin O

SPF—Specific Pathogen-Free

### REFERENCES

### References

Bachoud-Levi A C et al., Lancet (2000) 356
Bain G et al., Dev Biol (1995) 168:342-357
Basic Cell Culture Protocols (Pollard and Walker, eds.), Humana Press, 1997
Bjornson C R R et al., Science (1999) 283:534-537
Black I et al., J Neurosci (2000)
Bond J A et al., Int J Cancer (1996) 67:563-572
Boukamp P, Semin Cell Biol (1995) 6:157-163
Bouwens L, J Pathol (1998b) 184:234-239
Bouwens L, Microsc Res Tech (1998a) 43:332-336
Brown P D and Benya P D, J Cell Biol (1988) 106:171-179
Brustle O et al., Science (1999) 285:754-756
Cibelli J et al., Science (2001) in press
Clark M S et al., J Neurosci (1995) 15:6167-6178
Cohen E et al., J Cell Sci (1999) 112:2657-2666
Deacon T et al., Nature Med (1997) 3:350-353
Eguchi G and Kodama R, Curr Opin Cell Biol (1993) 5:1023-1028
Fernandez-Valle C et al., J Neurosci (1997) 17:241-250
Fraichard A et al., J Cell Sci (1995) 108:3181-3188
Freeman T B et al., Proc Natl Acad Sci USA (2000) 97:13877-13882
Fuchs E and Segre J A, Cell (2000) 100:143-155
Gundersen H J G et al., APIMS (1988) 96:379-394
Hallows K R and Frank R C, Biorheology (1992) 29:295-309
Hamano T and Asofsky R, J Immunol (1984) 132:122-128
Hay E D and Zuk A, Am J Kidney Dis (1995) 26:678-690
Hoerger T J et al., Pharmacokinetics (1998) 14:541-557
Hohn H P and Denker H W, Exp Cell Res (1994) 215:40-50
Holtzer h et al., Proc Natl Acad Sci USA (1975) 72:513-517
Hynes M and Rosenthal A, Neuron (2000) 28:11-14
Imaizumi T et al., Nature Biotech (2000) 18:949-953
Keller G M, Curr Opin Cell Biol (1995) 7:862-869
Kikyo N and Wolffe A P, J Cell Sci (2000) 113:11-20
Kishkina V B et al., Biull Eksp Biol Med (1983) 96:97-99
Kopyov O V et al., Cell Transplantation (1996) 5:327-337
Lang A E, Archives Neurol (2000) 57:1118-1125
Lee S H et al., Nature Biotech (2000) 18:675-679
Leno G H et al., Cell (1992) 69:151-158
Levit K et al., Health Affairs (1998) 17:99-110
Nathanson M A, Curr Top Dev Biol (1986) 20:39-62
Ng Y Y et al., Nephrol Dial Transplant (1999) 14:28602872
Okado H and Takahashi K, Proc Natl Acad Sci USA (1988) 85:6197-6201
Okado H and Takahashi K, J Physiol (1990) 427:583-602
Okado H and Takahashi K, J Physiol (1990) 427:603-623
Pairault J and Lasnier F, Biol Cell (1987) 61:149-154
Paterson F C and Rudland P S, J Cell Physiol (1985) 125:135-150
Piccini P et al., Nature Neurosci (1999) 2:1137-1140
Pittenger M F et al., Science (1999) 284:143-147
Pratt H P et al., Cell (1981) 26:279-292
Rosen D M et al., Exp Cell Res (1986) 165:127-138
Sato K et al., Rinsho Shinkeigaku (1991) 31:809-814
Schmidt V and Alder H, Cell (1984) 38:801-809
Schuldiner M et al., Proc Natl Acad Sci USA (2000) 97:11307-11312
Shea T B, Cell Biol Int Rep (1990) 14:967-979
Siderowf A D et al., Movement Disorders (2000) 15:439-455
Strutz F and Muller G A, Nephrol Dial Transplant (2000) 15:1729-1731
Studer L et al., Brain Res Bull (1996) 41:143-150
Studer L et al., Nature Neurosci (1998) 1:290-295
Tabar V and Studer L (1997) Transplantation into the adult rodent brain, in Current Protocols in Neuroscience (McKay R D and Gerfen C R, eds.), John Wiley & Sons Inc., New York
Takigawa M et al., Cell Differ (1984) 14:197-204
Tamai K et al., Dev Growth Differ (1999) 41:41-49
Theise N D et al., Hepatology (2000) 32:11-16
Ulloa L and Avila J, Eur J Neurosci (1996) 8:1441-1451
van der Loo W et al., J Immunol 91979) 122:1203-1208
Whetton-Goldstein K et al., JAGS (1997) 45:844-849
Wright W E, J Cell Biol (1984) 98:427-435
Wu C F et al., J Neurobiol (1990) 21:499-507
Yan J et al., J Neurochem (2001) 76:307-311
Yang X et al., Biol Reprod (2000)
Yeomans N D et al., Gastroenterology (1976) 71:770-777
Yoshida M et al., BioEssays (1995) 17:423-430
Yuan S et al., Differentiation (1996) 61-:67-75
Yujiri T et al., J Biol Chem (1999) 274:12605-12610
Zanetti N C and Solursh M, J Cell Biol (1984) 99:115-123
Zawada W M et al. Nature Med (1998) 4:569-573

The invention will now be described in more detail with respect to the following, specific, non-limiting examples.

### EXAMPLES

### Example 1

### Fusion Protein Constructs

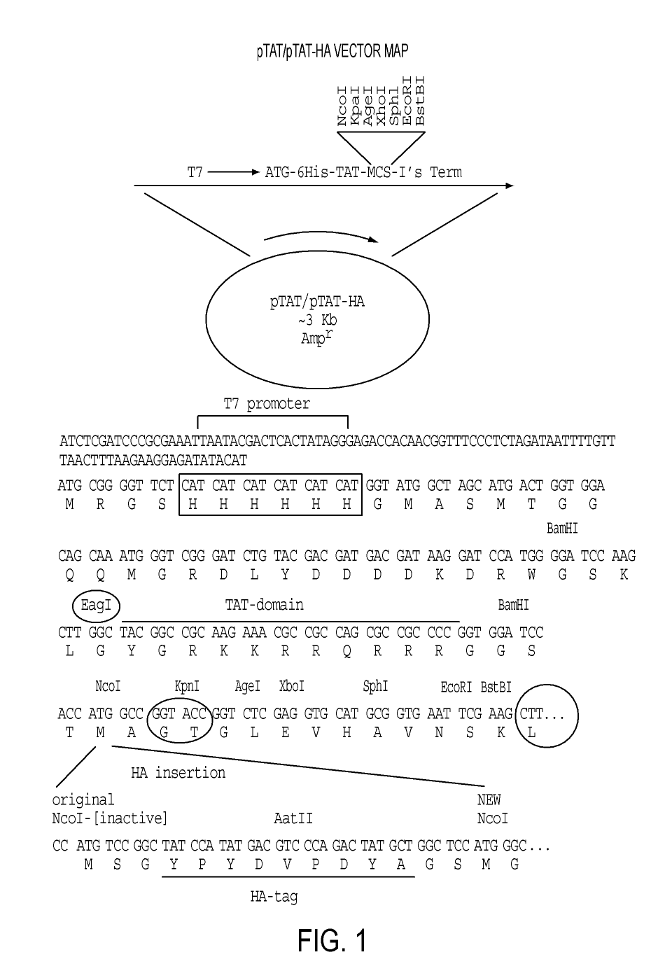

Expression vectors encoding reprogramming polypeptide were generated. The reprogramming polypeptides generated were human and mouse Oct4, Nanog, Klf-4, c-Myc, and Sox-2. Accession numbers for each gene are shown in Tables 1 and 2. To facilitate purification, detection, and introduction into recipient cells, the expression constructs included in-frame fusion to a protein transduction domain (PTD), an HA tag, and a 6×His tag. Human and mouse clones encoding the open reading frames were obtained from ATCC. The pTAT-HA-hOct4 and pTAT-HA-mOct4 expression vectors were generated by cloning PCR fragments encompassing the human and mouse Oct4 gene open reading frames into the NcoI and EcoRI sites of the pTAT-HA expression vector (FIG. 1). Vectors encoding Nanog, Klf-4, c-Myc, Sox-2, and Lin28 fusion proteins were cloned by essentially the same methods, with PCR products inserted into the pTAT-HA vector, resulting in the following constructs (with insertion sites noted parenthetically): pTAT-HA-hNanog (KpnI/EcoRI sites); pTAT-HA-mNanog (NcoI/EcoRI sites); c-Myc (NcoI/EcoRI sites); pTAT-HA-hSox-2 (KpnI/EcoRI sites); pTAT-HA-mSox-2 (KpnI/EcoRI sites); pTAT-HA-hKlf4 (NcoI/EcoRI sites); and pTAT-HA-mKlf4 (NcoI/EcoRI sites). PCR primers used for amplification of each open reading frame are shown in Table 6. The generated plasmids were confirmed by sequence analysis.

TABLE 6
PCR primers used to amplify target genes and generate fusion
constructs

| Primer | Sequence |
|---|---|
| Human Oct4 sense | 5′-TTCCATGGCGGGACACCTGGCTT-3′ |
| Human Oct4 | 5′-TTGAATTCTCAGTTTGAATGCATGGGAGAGC-3′ |
| antisense | |
| Mouse Oct4 sense | 5′-TTCCATGGCTGGACACCTGGCTTCA-3′ |
| Mouse Oct4 | 5′ -TTGAATTCTCAGTTTGAATGCATGGGAGAGC-3′ |
| antisense | |
| Human Nanog sense | 5′-ATACTGGTACCAGTGTGGATCCAGCTTG-3′ |
| Human Nanog | 5′-TTCACTCGAATTCACACGTCTTCAG-3′ |
| antisense | |
| Mouse Nanog sense | 5′-GAACGCCTCATCCATGGCTGCAGTTT-3′ |
| Mouse Nanog | 5′-CAGATGTTGCGGAATTCTCATATT-3′ |
| antisense | |
| c-Myc sense | 5′-CTCCCGCGACCATGGCCCTCAACGTT-3′ |
| c-Myc antisense | 5′-GACATTTCTGTTAGAAGGAATTCTTTT-3′ |
| Human Sox-2 sense | 5′-CGCCCGCATGGGTACCATGATGGAGA-3′ |
| Human Sox-2 | 5′-CTCCAGTTCGAATTCCGGCCCTCACA-3′ |
| antisense | |
| Mouse Sox-2 sense | 5′-TTTTTGGTACCATGTATAACATGATGGAGACG-3′ |
| Mouse Sox-2 | 5′-TTTTTGAATTCTCACATGTGCGAGAGGGGCA-3′ |
| antisense | |
| Human K1f4 sense | 5′-GCGAGTCTGCCATGGCTGTCAG-3′ |
| Human K1f4 | 5′-CACTGTCTGGAATTCAAAAATGCCT-3′ |
| antisense | |
| Mouse K1f4 sense | 5′-TTTTTCCATGGCTGTCAGCGACGCTCTGC-3′ |
| Mouse K1f4 | 5′-TTTTTGAATTCTTAATGCCTCTTCATG-3′ |
| antisense | |

### Example 2

### Purification of Recombinant Proteins Expressed in Bacterial Cells

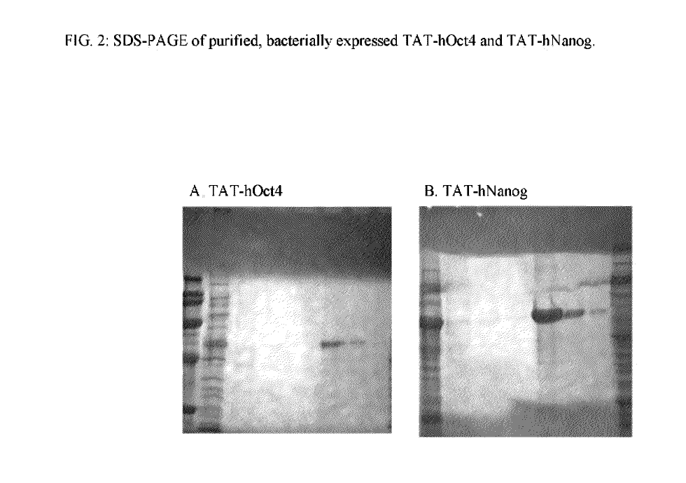

The plasmids pTAT-HA-hOct4 and pTAT-HA-mOct4, pTAT-HA-hNanog, or pTAT-HA-mNanog were each transformed into E. coli strain BL21(DE3)pLysS (Invitrogen), which contains an IPTG-inducible gene for T7 RNA polymerase. Fusion protein expression was induced by the addition of 1 mM IPTG at 30° C. for 4 h. The 6×His-fused recombinant proteins were observed to be sequestered into inclusion bodies by the host bacteria. To obtain purified protein, cells were disrupted by sonication in denaturation solution (6 M guanidinium, 20 mM NaPO₄, and 0.5 M NaCl, pH 7.8) and the 6×His-fused recombinant proteins were then bound to nickel resins (ProBond resin, Invitrogen). After several washings, the fusion proteins were eluted in 20 mM NaPO₄, pH 4.0, 0.5 M NaCl, 8 M urea plus 100 mM imidazole. The purity and concentration of the fusion proteins were determined by SDS-PAGE gel electrophoresis and visualized with Coomassie blue staining (FIG. 2). Both TAT-Oct4 and TAT-Nanog were very insoluble in native aqueous solution, but were soluble in 6-8 M urea.

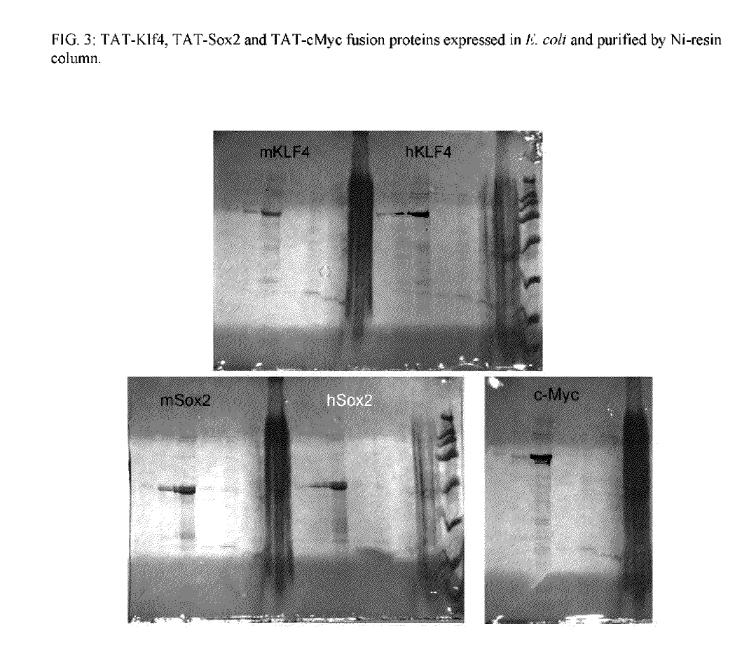

Essentially the same methods were utilized to express the Klf-4, c-Myc, Sox-2, and Lin28 fusion proteins. Unlike the Oct4 and Nanog constructs, these proteins did not form inclusion bodies and were soluble in native aqueous solution. These fusion proteins were purified by Ni-resin column and analyzed by SDS-PAGE and visualized with Coomassie blue staining (FIG. 3).

### Example 3

### Treatment of ES Cells with TAT-Oct-4

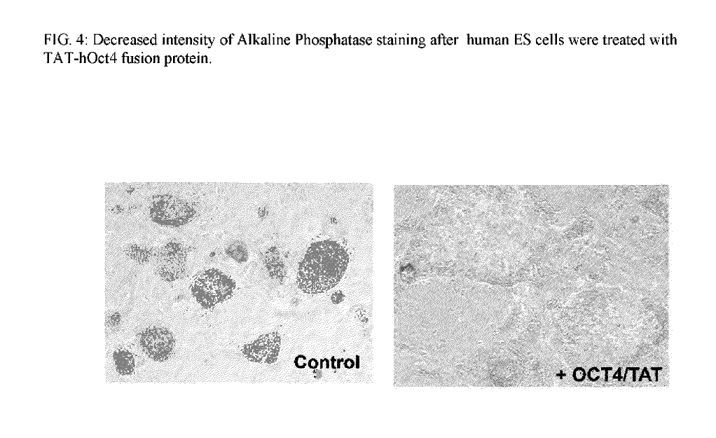

To test the hypothesis that addition of reprogramming proteins could help maintain stem cell lines in an undifferentiated state, the effect of TAT-Oct-4 on human ES cells was then tested. ES cells were grown under standard conditions, and the purified TAT-Oct-4 generated in Example 2 was added to the culture medium (the TAT-Nanog protein was not tested due to its poor solubility). The ES cells were then returned to a CO2 incubator and visually monitored. The ES cell colonies expanded and showed morphological signs of differentiation. Differentiation was confirmed by Alkaline Phosphatase (AP) staining. The TAT-Oct-4 treated cells showed diminished AP staining intensity relative to control human ES cell colonies (FIG. 4).

These results indicate that addition of TAT-OCT-4 alone may be insufficient to maintain ES cells in an undifferentiated state and suggests that a combination of reprogramming proteins would be more efficacious.

### Example 4

### Purification and Purification of Recombinant Reprogramming Proteins from Mammalian Cells

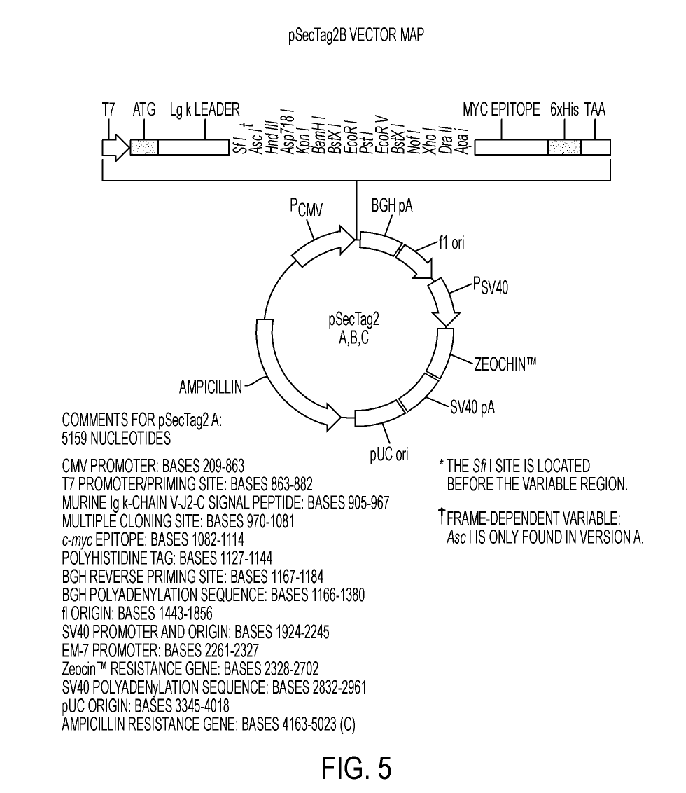

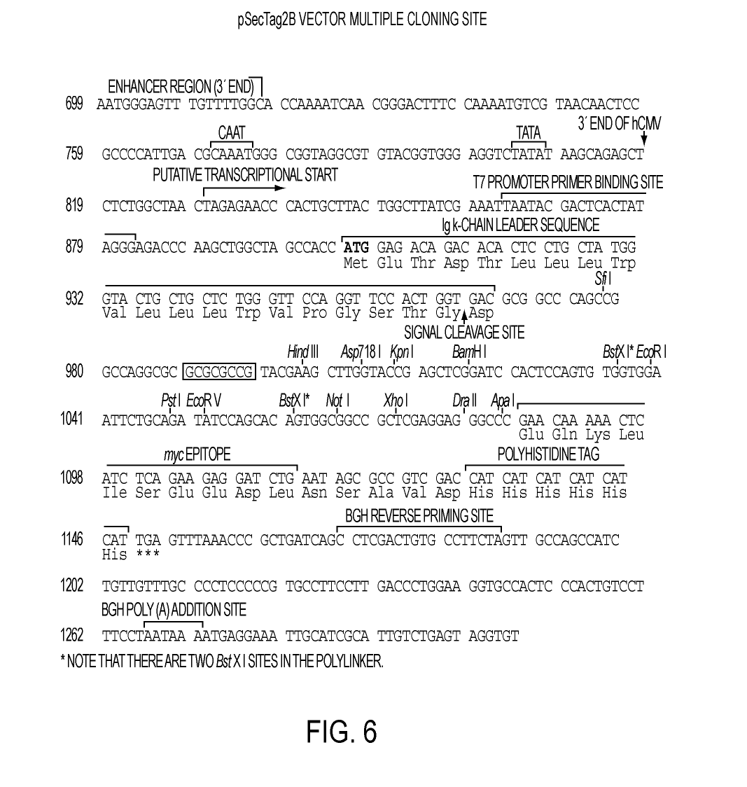

As described above, the Oct4 and Nanog fusion proteins were poorly soluble when purified from a bacterial expression system. To improve solubility, the human proteins were expressed in mammalian cells. The expression constructs described above were subcloned into the mammalian expression vector pSecTag2B (Invitrogen) (FIGS. 5 and 6), which contains an N-terminal secretion signal (Ig κ leader) expected to facilitate purification of the expressed fusion proteins. The TAT-HA-hOct-4 and TAT-HA-hNanog cDNAs from the pTAT-HA-hOct-4 and pTAT-HA-hNanog constructs were released by restriction enzyme digestion with Hind III and EcoRI, separated and purified by agarose gel electrophoresis, then recloned into the corresponding sites of pSecTag2B vector. The identities of the cloned genes were confirmed by sequence analyses. The constructs were then transfected into 293 cells and positive cells were selected with Zeocin. Despite the presence of the secretion signal, the expressed fusion proteins were not secreted into culture medium, but instead translocated into the nucleus. Nuclear localization was also observed for a GFP protein expressed from pSecTag2B (not shown).

Cell extracts were made essentially as previously described (Agarwal S., Methods Enzymol. 2006; 420:265-83) but without the addition of protease inhibitors. Recombinant proteins were prepared in 293 cell conditioned medium, whole cell lysates, nuclear lysates, and cytoplasmic lysates, and immunoprecipiated with HA-agarose. The proteins were then eluted with 200 mM glycine, pH 2.2, followed by neutralization with 1M Tris, pH 8.0.

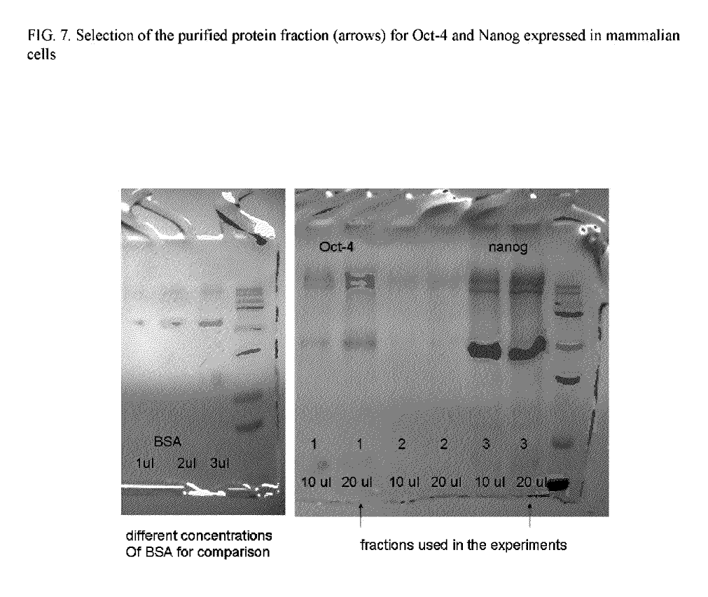

Fusion protein concentrations were determined by comparison of the stained electrophoresis gels to known amounts of a BSA stock solution (FIG. 7).

### Example 5

### Delivery of Recombinant Fusion Proteins into a Recipient Cell

Reprogramming protein fusion constructs (described in Examples 2 and 4) were delivered into recipient cells using a protein transfection reagent. The recipient cells were neonatal human dermal fibroblasts (NHDF) and mouse embryonic fibroblasts (MEFs), which were grown in 24-well cell culture plates (BD Biosciences, San Jose, Calif.; Corning, Lowell, Ma.) until approximately 50-70% confluence and exposed to the protein delivery-fusion protein mixture according to the manufacturers' instructions for 1-3 hours. The medium was then changed to DMEM with 10% FBS. Alternatively, cell suspensions were mixed with the protein-protein delivery reagent for 2 h, after which the cells were plated in the above medium. Cell samples were fixed and stained at different time intervals to detect entry of the fusion proteins into the cells. The reprogramming proteins used were Oct4, Nanog, Lin28, KLF4, c-myc, and Sox2, which were introduced into cells singly and in various combinations with one another.

One transfection reagents used for protein delivery was PULSin™ (Polyplus Transfection, distributed by Genesee Scientific, 8430 Juniper Creek Lane, San Diego, Calif. 92126). According to its manufacturer, PULSin™ contains a proprietary cationic amphiphile molecule that forms non-covalent complexes with proteins and antibodies. Complexes are internalized via anionic cell-adhesion receptors and are released into the cytoplasm where they disassemble. The process is non-toxic and delivers functional proteins. PULSin™ was used in accord with the manufacturers' instructions, which are as follows:

Per well in 24 well-plate:

a) Dilute between 0.5 μg and 4 μg of protein in 100 μl of 20 mM Hepes in a microcentrifuge tube. Vortex gently and spin down briefly.

b) Add between 0.5 μl and 4 μl of PULSin™ (in some experiments in which a combination of proteins was transfected into cells, PULSin™ was increased to 4 μl to 8 μl). Vortex immediately and spin down briefly.

c) Incubate for 15 minutes at room temperature.

d) Wash cells once with 1×PBS or culture medium without serum. The washing step is critical to remove all traces of serum.

e) Add 900 μl of culture medium without serum.

f) Add 100 μl of PULSin™/protein mix per well and homogenize by gently swirling the plate.

g) Incubate at 37° C. in a 5% CO2 incubator.

h) After 4 hours, remove the medium containing the biomolecules/PULSin™ complexes and replace with fresh complete medium.

i) Analyze the cells immediately or after an incubation period. Delivery can be analyzed by assessing protein activity or visualizing intracellular fluorescence.

Another transfection reagents used for protein delivery was SAINT-PhD (Synvolux Therapeutics B.V., L. J. Zielstraweg 1, 9713 GX Groningen, The Netherlands). According to its manufacturer, SAINT-PhD consists of a proprietary cationic pyridinium amphiphile and a helper lipid. Upon mixture of SAINT-PhD with the protein a particle of approximately 200 nm in diameter is formed. In this particle the protein is enwrapped by at least one bilayer of lipids. Furthermore, in the complex formed only non-covalent interactions are present between SAINT-PhD and the protein. The cationic amphiphiles on the surface of the particle have high affinity for the negatively charged cell surface. Upon fusion or entrapment of the particle the protein is released into the cytoplasm of the cell. The proteins delivered by SAINT-PhD are functional and unmodified. SAINT-PhD was used in accord with the manufacturers' instructions, which are as follows:

1. Dilute between 0.5 μg and 4 μg protein with the HBS (included in package) to a volume of 30 μL. If the protein is too diluted do not use HBS buffer at all.

2. Pipette between 10 μL and 304 SAINT-PhD into the protein/EMS solution.

3. After pipetting the SAINT-PhD the formulation may appear cloudy as complex formation occurs.

4. Add the prepared SAINT-PhD/Protein complex (step 2) directly to each well (drop-wise). Removal of growth medium is not necessary.

5. Swirl the plate gently to ensure an equally distribution over the entire plate surface.

6. Incubate at 37° C. in a 5% CO2 incubator.

7. Perform your assay after an appropriate incubation time.

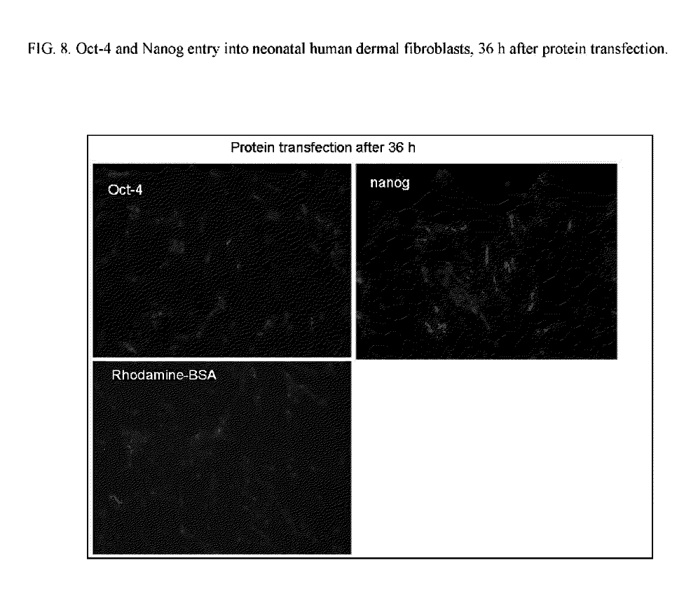

These experiments were performed to optimize the conditions of the protein delivery to accomplish efficient delivery of the proteins to the nucleus while maximizing cell survival. The fusion proteins were tested in concentrations between approximately 0.5 μg/ml and 4 μg/ml. After transfection, cell samples were fixed and analyzed by immunostaining with primary antibodies anti-Oct-2 and anti-Nanog (both from Santa Cruz Biotechnology, Inc., Santa Cruz, Ca.), each at 1:100 to 1:200 dilutions, which were detected using secondary biotinilated anti-mouse or anti-rabbit antibody (Jackson Immunoresearch; West Grove, Pa.; GE Healthcare (Amersham Biosciences)) followed by Streptavidin-FITC. Oct-4, Nanog, and rhodamine-labeled BSA were detectable in the cells, with positive immunostaining detected 48 h post transfection (FIG. 8).

The cells generally tolerated 3 hr procedure of protein delivery by the transfection reagent with between 1-4 μl of transfection reagent per reaction. The purified Oct4 and Nanog proteins (produced from 293 cells) were detectable in the recipient cells at higher levels than when added as unpurified extracts.

### Example 6

### Dedifferentiation of Cells by Contact with Recombinant Reprogramming Proteins

In this example, differentiated cells are reprogrammed by introduction of recombinant reprogramming proteins.

Neonatal human dermal fibroblasts (NHDF) are seeded in 24-well plates and grown to 50% to 70% confluence. Recombinant reprogramming proteins, each of which is genetically fused to a protein translocation domain (PTD) and/or nuclear localization signal (NLS), are added to the culture media to a final concentration of between approximately 0.1 and approximately 100 μg/ml. Concentrations of individual polypeptides and of combinations of polypeptides are determined in pilot experiments to be well tolerated by the cells (not causing unacceptably high levels of cell death) and preferably sufficient to result in detectable cell entry. In certain experiments, protein transfection reagents are also used to facilitate entry of the polypeptides into the cells. The cells are passaged onto mitotically inactivated MEFs and switched to hESC medium a few days after the first addition of reprogramming proteins.

Cell samples are periodically taken and the presence of the added reprogramming proteins in cell nuclei is monitored by immunofluorescence and Western Blotting, with the reprogramming proteins added as needed to maintain readily detectable levels.

The cells are visually monitored for the emergence of stem cell morphology. Cell samples are also periodically tested for expression of pluripotency markers by immunofluorescence, RT-PCR, and by radioactive metabolic labeling combined with IP/Western.

The recombinant reprogramming proteins include human Oct4, Nanog, Sox2, c-Myc, Klf4, and Lin28. A total of seventeen different combinations of reprogramming proteins are tested: one containing all six of these proteins, the six different combinations of five of these proteins, and the ten different combinations including Oct4 and three others. Duplicates of each combination are grown with the addition of valproic acid. Each combination is tested three to four times by the described methods. If reprogramming efficiency is unsatisfactory, variations on the protocol are tested, including sequential addition of reprogramming proteins instead of simultaneous addition, increasing the concentration of reprogramming agents used, increasing the frequency of addition of reprogramming agents, testing of other combinations of the six reprogramming proteins listed above, and testing of combinations that include reprogramming proteins listed in Tables 1 and 2, above.

When combinations that lead to successful reprogramming are identified, “leave one out” experiments are conducted to determine whether each individual constituent of the combination is required for the same efficiency of reprogramming.

Putative iPS cell lines prepared by these methods are then tested to confirm that they exhibit the expected properties, including: examining cell and colony morphology for the characteristic shape and appearance; long-term growth in culture (60-70 doublings) to confirm immortality; detection of telomerase activity; detecting increased levels (relative to the parental primary cell line) of pluripotency markers Alkaline Phosphatase, SSEA-1, Sox2, Oct4, Nanog, and Rex-1; detecting decreased DNA methylation in the promoters of the pluripotency genes Oct4 and Nanog; determination that global gene expression (by microarray) is more similar to ES and iPS cell lines than to the parental primary cell line; and detection of ability to differentiate in vitro and in vivo into cells in the three germ layers. Additionally, the cells are analyzed by G-banding and by spectral karyotyping to confirm the absence of genomic rearrangement, by PCR and Southern blotting to confirm the absence of undesired viruses and microorganisms (including testing for adenoviral and lentiviral sequences, and mycoplasma), and to confirm that the sequences encoding the reprogramming factors have not been inadvertently integrated into the iPS cell genome.

### Example 7

### Dedifferentiation of Human Donor Cells by Contact with Recombinant Reprogramming Proteins

A skin biopsy is taken from a human donor, washed, and cut into small fragments, which are distributed in the bottom of a tissue culture flask. Fibroblast culture media (DMEM with 10-15% FBS) is then added, and the cell flask is placed in a CO2 incubator and monitored, with culture media replaced every two to three days until fibroblasts are observed growing on the bottom of the tissue culture flask. Thereafter, the primary dermal fibroblasts are maintained using standard growth techniques. A frozen stock of the cultured primary dermal fibroblasts is kept for later use.

The primary dermal fibroblasts are then seeded in a tissue culture plate and grown to approximately 50-70% confluence. Reprogramming agents are then added in a combination, concentration, and frequency that is effective for reprogramming of fibroblasts (e.g., as is identified by the methods described in Example 6). Cells are monitored for the emergence of iPS cell colonies, which are then dispersed, passaged, and expanded to establish individual iPS cell lines.

As a result of this treatment, iPS cell lines derived from the human donor are obtained. Using the methods described in Example 6, the cell lines are then tested to confirm that they exhibit the expected properties of iPS cells, and tested to confirm the absence of genomic rearrangement, other undesired genome sequence changes, and pathogens or pathogenic sequences. Suitability for therapeutic uses such as transplantation into a patient is thereby confirmed.

### Example 8

### Dedifferentiation of Cells by Contact with Donor Cell Cytoplasm

A recipient cell is dedifferentiated in vitro by the introduction of donor cell cytoplasm. The recipient cells, neonatal human dermal fibroblasts (NHDF), are seeded in 24-well plates and grown to 50% to 70% confluence. Donor cell cytoplasm is introduced into different populations of recipient cells by recipient cell permeabilization with Streptolysin O (Agarwal S., Methods Enzymol. 2006; 420:265-83), by fusion with cytoplasmic blebs, by fusion with liposomes, and using a protein transfection reagent. Donor cell cytoplasm is periodically re-added to each recipient cell population, as frequently as the cells tolerate without excessive levels of cell death. The cells are visually monitored for the emergence of stem cell morphology.

The sources of donor cell cytoplasm are oocytes, blastomere cells, iPS cells, human ES cells, and 293 cells that have been caused to express a combination of reprogramming polypeptides that has been shown by the methods in Example 6 to be effective for reprogramming. Each type of cytoplasm is used with each of the aforementioned methods of introducing the cytoplasm into recipient cells. Additionally, duplicates of each combination are grown with the addition of valproic acid.

As a result of this treatment, iPS cell lines are obtained. Using the methods described in Example 6, the cell lines are then tested to confirm that they exhibit the expected properties of iPS cells, and tested to confirm the absence of genomic rearrangement, other undesired genome sequence changes, and pathogens or pathogenic sequences.

### Example 9

### Dedifferentiation of Human Donor Cells by Contact with Donor Cell Cytoplasm

Primary dermal fibroblasts are grown from a human donor as described in Example 7. Donor cell cytoplasm is then added with a concentration and frequency that is effective for reprogramming of fibroblasts (e.g., as is identified by the methods described in Example 8). Cells are monitored for the emergence of iPS cell colonies, which are then dispersed, passaged, and expanded to establish individual iPS cell lines.

As a result of this treatment, iPS cell lines derived from the human donor are obtained. Using the methods described in Example 6, the cell lines are then tested to confirm that they exhibit the expected properties of iPS cells, and tested to confirm the absence of genomic rearrangement, other undesired genome sequence changes, and pathogens or pathogenic sequences. Suitability for therapeutic uses such as transplantation into a patient is thereby confirmed.

### Example 10

### Introduction

This example describes methods for directing the reprogramming of permeabilized somatic cells by exposing them to nuclear and cytoplasmic extracts of the cell type desired, in vitro, while exposing them to inductive culture conditions. While not intending to be limited by theory, it is hypothesized that every given cell type contains the key regulatory factors (including transcription factors) that determine it's gene expression profile and identity; thus, exposing one type of cell to regulatory factors derived from a different type of cell can redirect the gene expression pattern and identity toward a different type of cell. Additionally, this conversion can be promoted by specific inducing factors, cell culture conditions, Chromatin remodeling agents, and/or Transcription Modifiers. Unless stated otherwise, cell extract generation and recipient cell permeabilization are performed are essentially as described in Agarwal, “Cellular Reprogramming” (2006) Methods Enzymol. 420: 265-283 which is incorporated by reference herein in its entirety.

Methods

Cell extracts are introduced into a recipient cell by reversible permeabilization of recipient cells using the pore-forming, calcium sensitive bacterial toxin Streptolysin O (SLO) and exposure of these cells to reprogramming cell extracts. Limited and transient exposure of cells to low doses of SLO in the absence of calcium ions allows the formation of membrane pores that are large enough to allow the passive diffusion of proteins up to the size of 100 kilodaltons, but not large enough for organelles. Subsequently, reversal of this membrane permeabilization by adding calcium ions allows the membrane to repair itself and the resealed cells are viable and can proliferate. During the permeabilization process, the target cell can be incubated with whole cell extracts or nuclear or cytoplasmic extracts of a desired cell type (“donor cell”), and optionally in the presence of permeabilization/reprogramming promoting agents such as an energy generating system and cytoskeletal disruptors. The whole cell extract can provide regulatory factors that are taken up by the permeabilizing target cell. The plasma membrane is then resealed, the cells are allowed to recover and cultured further, in conditions conducive to desired reprogramming. Consequently, gene expression profiles of the recipient cells become altered to more closely resemble the donor cells. The resultant changes in gene expression profiles may arise over time, and may be further promoted by subsequent rounds of treatment with donor cell extract.

Fluorescently conjugated proteins (70 kDA Rhodamine-dextran or 68 KDa Rhodamine-albumin) can be used to conveniently monitor the efficiency of cell permeabilization and uptake of exogenous factors. We found that the efficiency of SLO mediated membrane permeabilization and uptake of proteins can be sensitive to several factors including the density of the cells, use of adherent versus suspension cell cultures, the concentration of SLO, SLO activation, length of exposure to SLO and exogenous factors, the quality of the cell extracts (if cell extracts are used), and time given for resealing of membrane pores and recovery. These factors can be routinely optimized for a given cell type.

Reprogramming Cell Extract Generation

Cultures of hES cells or control 293T cells were healthy, exponentially growing cells. Confluent or overgrown cultures were not used.

Cells were then harvested, washed with PBS (without calcium and magnesium) two times with sedimentation between washes by centrifugation (1000 rpm for 5 minutes in a swinging bucket rotor). Care was taken to fully aspirate wash media to remove serum proteins or calcium ions that could potentially interfere with subsequent use in cell permeabilization and reprogramming reactions. After washing, cell pellets were optionally snap frozen in liquid nitrogen and stored ad −80 degrees C. From this point forward, cells and extracts thereof were maintained on ice.

Cell pellets were resuspend by pipetting in 1-2 volumes of freshly prepared, ice-cold lysis buffer (20 mM HEPES, pH 8.2, 5 mM MgCl2, 1 mM DTT, 1× protease inhibitors): prepared fresh and kept on ice.) Cells were then incubated on ice for 1 h. This incubation of the cells in the hypotonic lysis buffer will cause them to swell, which facilitates their disruption during sonication. Cells were then sonicated in short pulses until lysed, using a Fisher Scientific Sonic Dismembrator, Model 100. The probe of the sonicator was kept sterile by cleaning with water and alcohol and washing before shifting between different types of cells. The power and time of sonication may vary between cell types and may be routinely determined by monitoring the extent of lysis (e.g. microscopically). If multiple pulses are required for a given cell type, the cells are cooled on ice between pulses to avoid unnecessary heating. Complete sonication can be judged by a loss in the viscosity of the lysate. The lysate can also be examined under the microscope for loss of intact cells and nuclei.

The sonicated cell lysate was then transferred to 1.5-ml microcentrifuge tubes (if not already in such tubes), then snap-frozen in liquid nitrogen, followed by a quick thaw in a 37 degree C. water bath to fragment any remaining genomic DNA, then centrifuged for 15-30 min at 4 degrees in a fixed-angle microcentrifuge at 14,000 rpm. Supernatant was then aliquoted in 200-500 μl volumes and stored at −80 degrees C.

Protein concentration of the cell extract was then determined. Typically, we obtained concentrations of 6-9 mg/ml. For the reprogramming reactions, we have typically used the cell extracts between 1.5-6 mg/ml. Toxicity of a given extract to a given recipient cell type can be determined by routine experimentation and may limit the use of higher extract concentrations.

Additionally, quality of the extract may be determined by electrophoresis and inspecting the general protein profile by Coomassie stain to ensure that there is no visible protein degradation. Presence of cell type-specific proteins (nuclear and cytoplasmic) in the cell extracts may also be verified by immunoblotting. For example, proteins that are known to have critical “master regulatory” roles in a particular cell type can be examined.

Recipient Cell Permeabilization and Treatment with Reprogramming Extract

Prior to use, SLO was activated by incubation with a reducing agent as described by Agarwal (p. 272). Exponentially growing cultures of recipient 293T cells (which had previously been acclimated to hES cell growth media) were detached from the culture dish by trypsinization and permeabilization was performed in suspension, as this had been determined to improve efficiency of uptake. Cells were washed, counted, and aliquots of 1-3×10̂5 cells were incubated per reaction. Care was taken to process the cells before clumps could form. Recipient cells aliquots were precipitated at room temperature (1000 RPM for 5 min. in a swinging bucket rotor). Then to each cell pellet, the following were added in order (reaction tubes were gently tapped intermittently during these additions to prevent settling of the cells).

a. Reprogramming cell extract or control extract (1.5-6 μg/l): 73 μl. Preparation of the reprogramming cell extract is described above

b. 0.5 M EDTA: 2 μl (final concentration: 10 mM)

c. 0.1 M MgCl₂: 5 μl (final concentration: 5 mM)

d. 25 mM NTP stock mix: 4 μl (final concentration: 1 mM each NTP)

e. 1.5 mg/ml Creatine kinase: 1.5 μl (final concentration: 25 μg/ml)

f. 1 M Phosphocreatine: 1 μl (final concentration: 10 mM)

g. 100 mM ATP: 1 μl (final concentration: 1 mM)

h. 10 mM GTP: 1 μl (final concentration: 100 μM)

i. 2 mg/ml Cytochalasin B: 0.75 μl (final concentration: 15 μg/ml)

j. To each individual reaction tube, add 10 μl of 0.5 units/μl activated SLO (final SLO concentration: 50 units/ml). As noted above, efficiency of SLO-mediated permeabilization varies among cell types, and the optimum concentration of SLO was determined in pilot experiments using fluorescence conjugated marker proteins (rhodamine-labeled dextran (70 kDa) or rhodamine-labeled albumin (68 kDa), final concentration 10-50 μg/ml) as tracers and may optionally be included with the cell extracts or as positive controls for confirmation of uptake. After resealing of the recipient cell membrane, uptake of the fluorescent proteins can be assessed by fluorescence microscopy or other known methods to indicate the efficiency of cell permeabilization, resealing of the membrane and cell survival.

The reaction mixtures were then incubated at 37 degrees C. for 3 h. To prevent the cells from settling or clumping during this incubation, we used a gentle rocker during incubation.

After the incubation, all the contents of each reaction were gently pipetted and dilute in 0.75 ml of hES cell culture media in one well of a 4-well or 24-well culture dish on mitotically inactivated MEFs. The cell culture media contained at least 2 mM CaCl2 to initiate resealing of the permeabilized cell membrane.

Cells were then incubated overnight at 37 degrees C. in standard culture conditions to permit recovery; then media was aspirated (including any unattached dead cells) and replaced with fresh media. Cultures were then maintained under standard culture conditions in hES cell media. Optionally cells may be subjected to a second or subsequent round of permeabilization and extract treatment as described above.

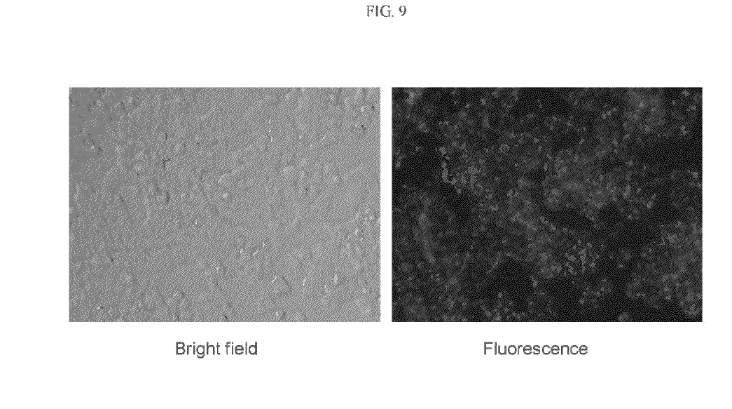

FIG. 9 depicts robust uptake (90-100%) of Rhodamine-albumin in SLO permeabilized human fibroblasts, 293T cells following optimization of permeabilization conditions. Optimized treatment conditions were determined for primary human neonatal dermal fibroblasts (NHDF cells) and mouse NIH 3T3 fibroblasts. In each case, the cells take up the fluorescent protein robustly, recover and grow to confluence and appear to retain and partition the input protein through successive doublings.

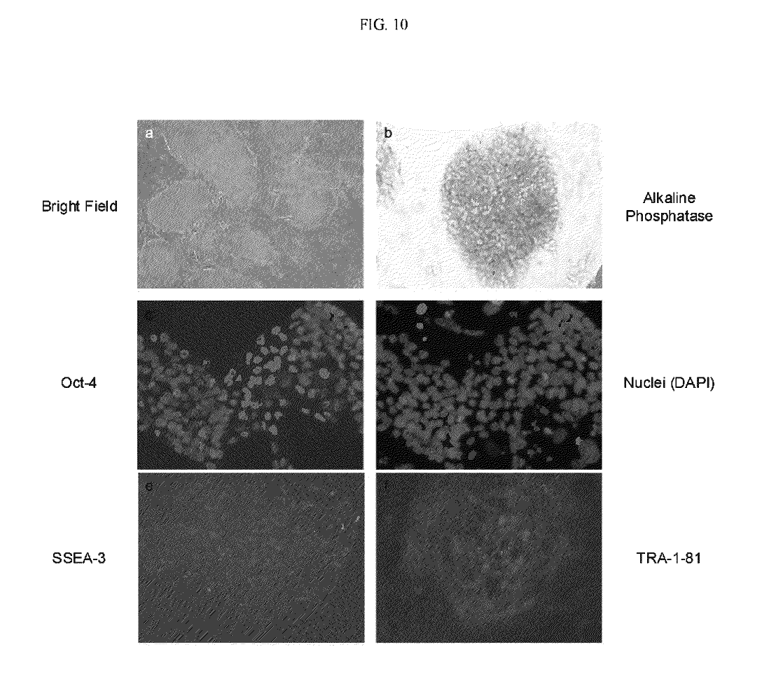

hES cell extracts were obtained from the hES cell lines H9 (WA09) and ACT4. To guard against spontaneous differentiation, cells were not allowed to overgrow; moreover, cultures were periodically examined for hES cell morphology and expression of characteristic markers of hES cells. FIG. 10 depicts an example of a typical hES cell culture characterization: (a) phase contrast microscopy; (b) alkaline phosphatase activity assay; and immunofluorescence for the expression of hES cell markers (c) Oct-4 (e) SSEA-3, and (f) Tra-1-81, as indicated. Panel (d) depicts the DAPI stain for nuclei of the same field as stained for Oct-4 in (c). As shown, the hES colonies exhibit typical undifferentiated cell morphology, score positive for alkaline phosphatase activity, and express the characteristic hES cell markers Oct-4, SSEA-3 and TRA-1-81, as determined by immunofluorescence. The cells express robust amounts of the hES cell pluripotency marker and key transcription factor, Oct-4.

Results

Permeabilized 293T fibroblast cells were incubated with hES cell extracts in conditions of hES cell culture. Prior to treatment, the 293T cells were adapted to ES cell culture medium. Recipient cells were incubated with hES cell whole cell extracts, SLO, an ATP generating system and the cytoskeleton disrupter Cytocholasin B. Control cells were treated in parallel with 293T cell (“self”) extracts. Post incubation, the cells were plated in conditions of hES cell growth, i.e. in hES cell culture medium and on mitogenically inactivated mouse embryonic fibroblast (MEF) feeder layers.

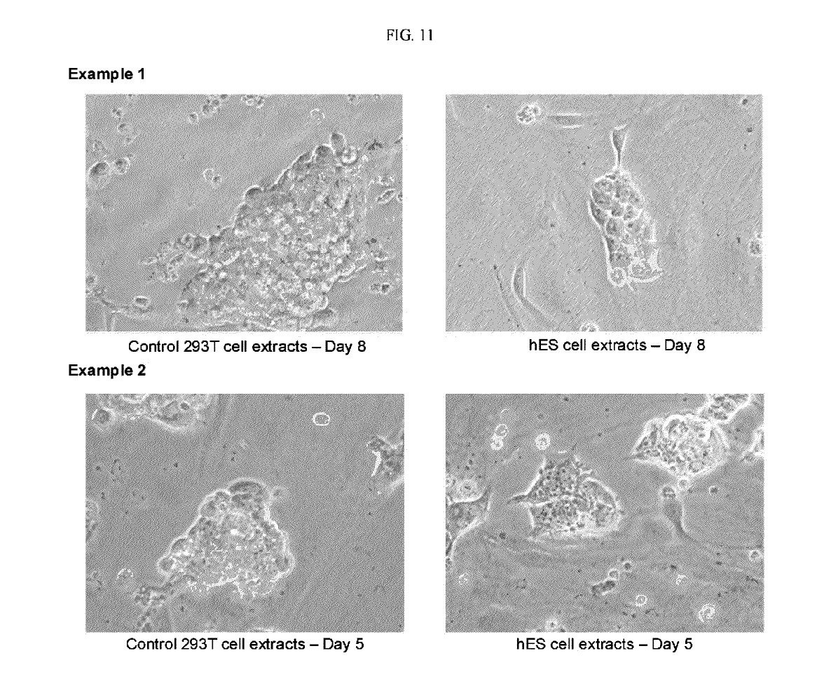

Microscopic examination revealed cell colonies growing on the MEF layers after treatment. Colonies were observed with both the hES cell extract treatment and control treatment, however, the colonies obtained with hES cell extracts had a more hES cell-like appearance than controls (FIG. 11). The colonies obtained with control extracts tended to appear multilayered, with smaller, more rounded cells piling on top of each other (FIGS. 11A and 11C). In contract, the colonies obtained with hES cell extract appeared flatter, single layered with large nucleus to cytoplasmic ratios and distinct nucleoli and visible sub-nuclear structures (“specks”) characteristic of hES cells (FIGS. 11B and 11D). Similar results were obtained in two experiments (first experiment, 11A-B; second experiment, 11C-D).

Resulting cells are maintained and expanded in culture and optionally are treated with hES cell extracts a second or subsequent time. Due to toxicity of SLO treatment cells may be permitted to recover for a time between treatments, or experiments may be performed with greater numbers of cells to permit recovery of greater numbers of viable cells subjected to multiple rounds of treatment.

Treated cells are monitored for ability to form colonies on MEFs that have morphological characteristics of hES cells described above. Additionally, cells having such morphology are tested for expression of hES cell markers by immunofluorescence, RT-PCR, or other known methods of detecting gene expression. Such hES cell markers may include Oct4, Nanog, Sox2, SSEA-3 and Tra-1-81. Cells may also be tested for alkaline phosphatase activity, a marker of ES cells. Cells may also be tested for pluripotency by determining the ability to give rise to differentiated cells of different types in vitro or in vivo (e.g., teratoma formation in immunocompromised animals; giving rise to progeny cells following blastocyst injection; or giving rise to whole progeny animals following tetraploid complementation). Cells may also be tested for loss of methylation in the promoters of the Oct4 and Nanog genes, indicating reactivation of expression of these genes. Global gene expression patters may also be examined by microarray methods and compared to expression profiles of existing ES cell lines, where similarity further confirms reprogramming to form ES cells.

### Example 11

Bovine fetal fibroblasts (BFFs) were grown to confluence and seeded onto 100 mm plates at approximately 250,000 cells/plate. Cells were grown in DMEM (Gibco) supplemented with 0.03% L-Glutamine (Sigma), 100 μM non-essential amino acids (Gibco), 10 units/L Penicillin-Streptomycin (Gibco), 154 μM 2-Mercaptoethanol (Gibco) and 15% FBS (HyClone). Four treatments were used:

1. A control grown in the medium described above,

2. DMEM with 2.5 μg/ml CB,

3. DMEM with 5.0 μg/ml CB, and

4. DMEM with 7.5 μg/ml CB.

Control cells were grown in the presence of DMSO alone to evaluate its effect on priming and trans-differentiation.

BFFs cultured in treatment 1 began to rapidly divide and grow to confluence as was expected. BFFs cultured in treatment 2 did not undergo cytokinesis, however did undergo nuclear division leading to multinuclear fibroblasts. BFFs cultured in treatments 3 and 4 began to change morphology and by day 2 of treatment began to take on the appearance of neuronal cells. On day 3 of treatment, a small population of cells that had been grown on glass cover slips were fixed from each of the described treatments, and incubated with an antibody to tyrosine hydroxylase (the rate limiting enzyme involved in dopamine production, specific to neuronal cells). Cells were visualized under fluorescence for detection of antibody labeling. Control cells did not exhibit fluorescence, and cells from groups 2, 3, and 4 fluoresced in a dose-dependent manner, which correlated directly with increasing amounts of CB.

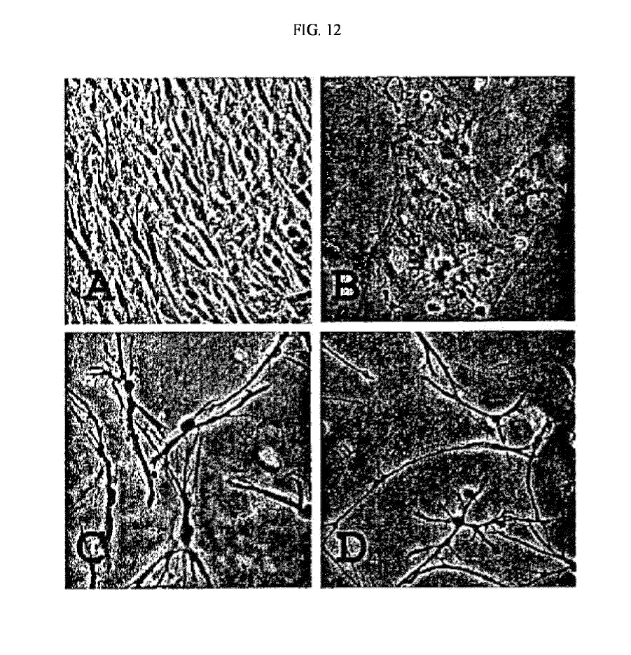

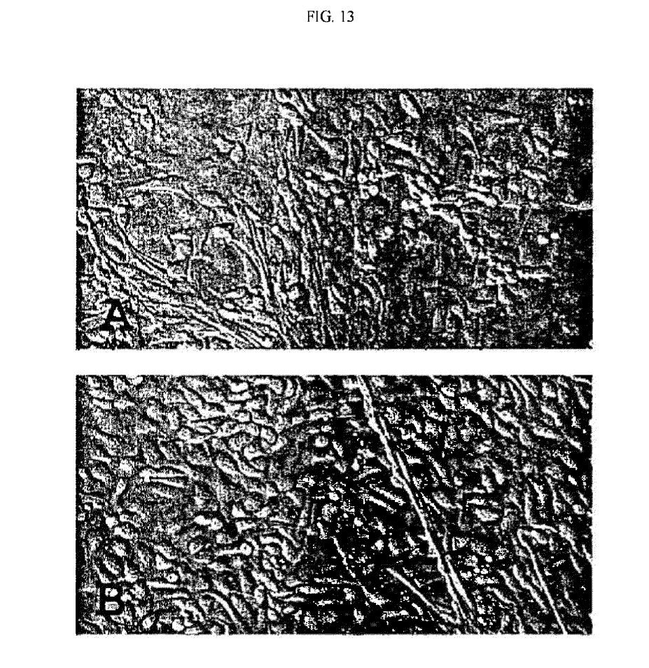

In conclusion, treatment of BFFs with CB at 2.5-7.5 μg/ml is effective at inducing bovine fetal fibroblasts to undergo morphological changes toward a neuronal-like lineage as well as inducing the expression of tyrosine hydroxylase. These results suggest that trans-differentiation can be primed by microfilament inhibitors. Our preliminary data suggest that virtually all the primary fibroblasts (millions from a single patient sample) can be primed within 12-24 hours of in vitro culture. Results are presented in FIGS. 12 and 13.

### Example 12

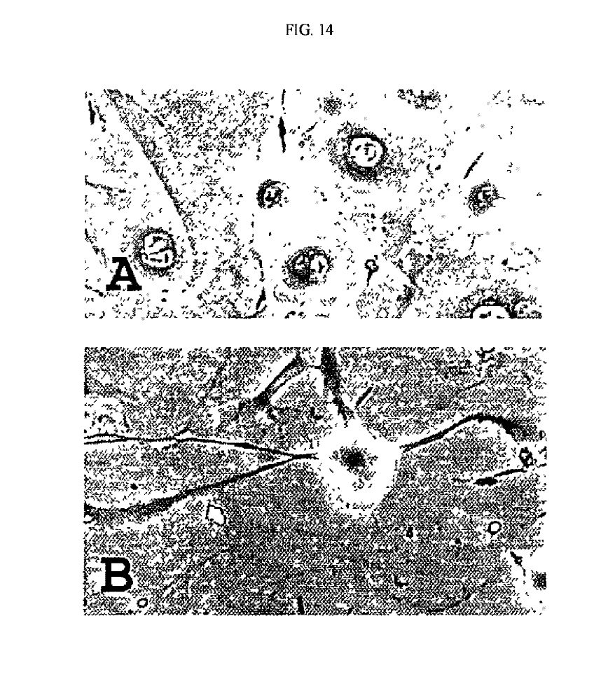

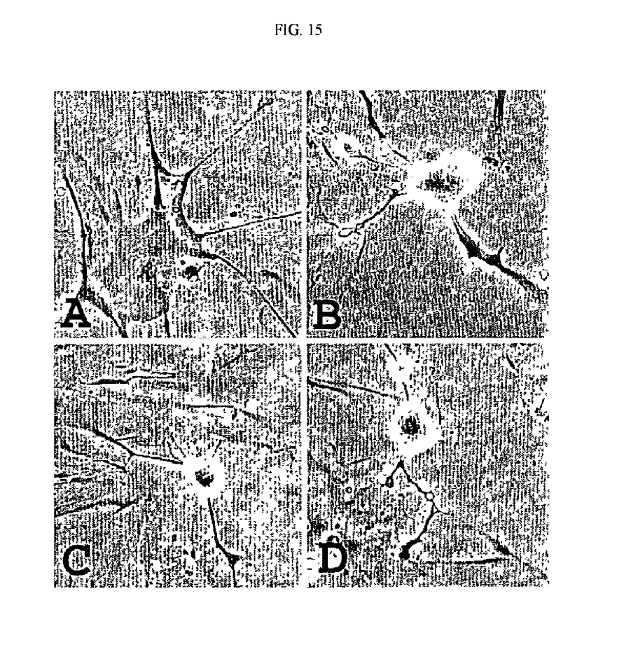

Bovine adult fibroblasts (BAFs) were treated in the manner described for BFFs in Example 11, with priming carried out using 10.0 μg/ml CB for 72 hours. Like BFFs, treatment of the BAFs with the priming agent and culturing them under conditions that induce neural differentiation caused the cells to undergo morphological changes toward a neuronal-like lineage. See FIGS. 14 and 15. Note that BFFs and BAFs acquire different morphologies of a neural type.

### Example 13

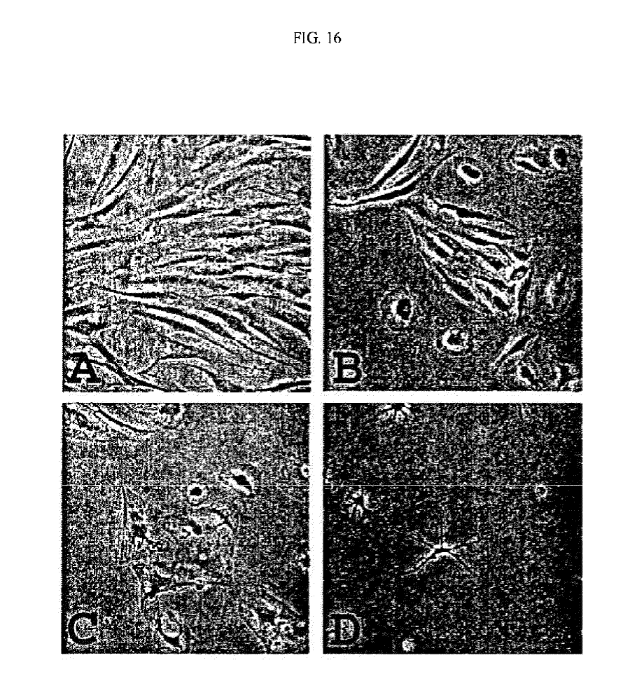

Transdifferentiation of human neo-natal fibroblasts. Fibroblasts were purchased from Cambrex company (Clonetics cell line #CC-2509) and were expanded in Iscove's Modified Dulbecco's Medium (IMDM, Gibco) supplemented with 20% fetal bovine serum (HyClone) at 37 degrees C. in 5% CO₂ and 5% O₂. At passage 14, cells were weaned from serum by replacing medium every other day with half the concentration of serum over a 2-week period. When cells had been in the absence of serum for 48 hours, they were seeded at 50% confluency in 24-well dishes. 24 hours after passage, IMDM was removed and replaced with conducive medium (keratinocyte growth medium (KGM, Clonetics) was added to half of the cultures and neuro-progenitor growth medium (NPMM, Clonetics) was added to the other half). After 24 hours in conducive medium, 5 μg/ml cytochalasin B (CB, CalBiochem) was supplemented into half of each media group. Cells were cultured for an additional 72 hours at which point half of all groups were fixed in 4% paraformaldehyde (Sigma) in Dulbecco's phosphate-buffered saline (DPBS, Biowhittaker), and the remaining half were replaced with fresh medium (KGM and NPMM respectively) without CB. These cells were then cultured for another 72 hours at which point they were fixed in 4% paraformaldehyde in DPBS. As with BFFs and BAFs, treatment of the human fetal fibroblasts BFFs with CB at 5 μg/ml and culturing them under conditions that induce neural differentiation is effective at inducing the fibroblasts to undergo morphological changes toward a neuronal-like lineage (see FIG. 16).

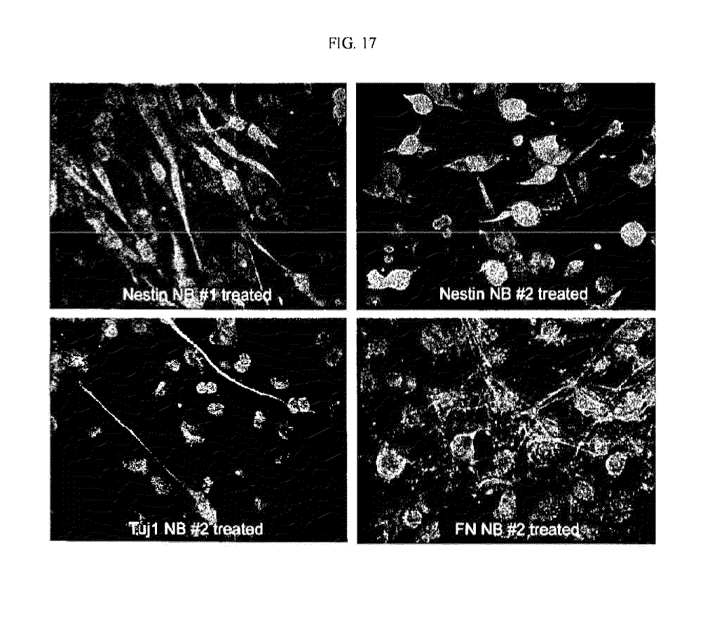

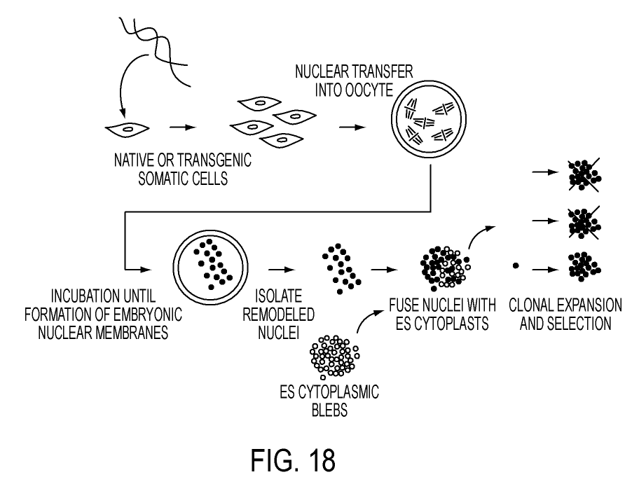

Immunocytochemistry was conducted using antibodies to Nestin, Glial Fibriliary Acid Protein (GFAP), Oligo 4 (O4), beta Tubulin III, Tuj1, Gamma Amino Buteric Acid (GABA), Tyrosine Hydroxylase, MAP2ab, Calretinin, Tropomyosin. Cells treated with cytochalasin B were positive for markers of cells of neuronal lineage-nestin, Tuj-1, and beta tubulin III (see FIG. 17). The control fibroblasts not treated with CB were negative for all markers. Nestin is an intermediate microfilament present in neural stem cells prior to terminal differentiation. Tuj-1 is a neuron-specific tubulin, and beta Tubulin III is a microtubule that is present only in neurons.

### Example 14

### Nuclear Remodeling

The first step (also referred to herein as the “nuclear reprogramming step”) is performed using human peripheral blood mononuclear cells which are purified from blood using Ficoll gradient centrifugation to yield a buffy coat comprised primarily of lymphocytes and monocytes as is well known in the art. The use of lymphocytes with a rearranged immunoglobulin locus as donors in the present method will result in stem cells with the same rearranged loci. In the case where the desired outcome of the experiment is not cells with a preformed rearrangement in immunoglobulin genes, the monocytes are purified from the lymphocytes by flow cytometry as is well known in the art and stored at room temperature in Dulbecco's minimal essential medium (DMEM) or cryopreserved until use. Xenopus oocytes from MS222 anesthetized mature females are surgically removed in MBS buffer and inspected for quality as is well-known in the art (Gurdon, Methods Cell Biol 16:125-139, (1977)). The oocytes are then washed twice in MBS and stored overnight at 14 degrees C. in MBS. The next day, good quality stage V or VI oocytes are selected (Dumont, J. Morphol. 136:153-179, (1972)) and follicular cells are removed under a dissecting microscope in MBS. After defolliculation, the oocytes are stored again at 14 degrees C. overnight in MBS with 1 μg/mL gentamycin (Sigma). The next day, oocytes with a healthy morphology are washed again in MBS and stored in MBS at 14 degrees C. until use that day. Approximately 1×10̂4 monocytes are permeabilized by SLO treatment as described by Chan & Gurdon, Int. J. Dev. Biol. 40:441-451, (1996). Briefly, the cells are suspended in ice-cold lysis buffer (1×Ca2+-free MBS containing 10 mM EGTA (Gurdon, (1977)]. SLO (Wellcome diagnostics) is added at a final concentration of 0.5 units/mL. The suspension is maintained on ice for 7 minutes, then four volumes of 1×Ca2+-free MBS containing 1% bovine serum albumin (Sigma) is added. Aliquots of the cells may then be removed, diluted 1× in 1×Ca2+-free MBS containing 1% bovine serum albumin, and incubated at room temperature for five minutes to activate permeabilization. The cells are then placed back on ice for transfer into the Xenopus oocytes. The permeabilized cells are then transferred into Xenopus oocytes as is well known in the art (Gurdon, J. Embryol. Exp. Morphol. 36:523-540, (1976). Briefly, oocytes prepared as described above are placed on agar in high salt MBS (Gurdon, J. Embryol. Exp. Morphol. 36:523-540, (1976)). The DNA in the egg cells is inactivated by UV as described (Gurdon, Methods in Cell Biol 16:125-139, 1977) with the exception that the second exposure to the Hanovia UV source is not performed. Briefly, egg cells are placed on a glass slide with the animal pole facing up and are exposed to a Mineralite UV lamp for 1 minute to inactivate the female germinal vesicle. The permeabilized monocytes are taken up serially into a transplantation pipette 3-5 times the diameter of the monocytes and injected into the oocyte, preferably aiming toward the inactivated pronucleus. The egg containing the nuclei are incubated for one hour to 7 days, preferably 7 days, then removed and used in step 2. The oocytes may, if desired, be manipulated prior to use to alter the levels of one or more cell factors as described above.

### Example 15

### Nuclear Remodeling

In this example, step one of nuclear remodeling is carried out in an extract from undifferentiated cells of the same species as the differentiated cell; human dermal fibroblasts nuclei are remodeled in vitro using mitotic cell extracts from the human embryonal carcinoma cell line NTera-2. However, extracts from cells of a different species may alternatively be used.

Preparation of Nuclear Remodeling Extract

NTera-2 cl. D1 cells are easily obtained from sources such as the American Type Culture Collection (CRL-1973) and are grown at 37 degrees C. in monolayer culture in DMEM with 4 mM L-glutamine, 1.5 g/L sodium bicarbonate and 4.5 g/L glucose, 10% fetal bovine serum (complete medium). While in a log growth state, the cells are plated at 5×10̂6 cells per sq cm tissue culture flask in 200 mL of complete medium. Extracts from cells in the prometaphase are prepared as is known in the art (Burke & Gerace, Cell 44: 639-652, (1986)). Briefly, after two days and while still in a log growth state, the medium is replaced with 100 mL of complete medium containing 2 mM thymidine (which sequesters the cells in S phase). After 11 hours, the cells are rinsed once with 25 mL of complete medium, then the cells are incubated with 75 mL of complete medium for four hours, at which point nocodazole is added to a final concentration of 600 ng/mL from 10,000× stock solution in DMSO. After one hour, loosely-attached cells are removed by mitotic shakeoff (Tobey et al., J. Cell Physiol. 70:63-68, (1967)). This first collection of removed cells is discarded, the medium is replaced with 50 mL of complete medium also containing 600 ng/mL of nocodazole. Prometaphase cells are then collected by shakeoff 2-2.5 hours later. The collected cells are then incubated at 37 degrees C. for 45 minutes in 20 mL of complete medium containing 600 ng/mL nocodazole and 20 μM cytochalasin B. Following this incubation, the cells are washed twice with ice-cold Dulbecco's PBS, then once in KHM (78 mM KCl, 50 mM Hepes-KOH [pH 7.0], 4.0 mM MgCl₂, 10 mM EGTA, 8.37 mM CaCl2, 1 mM DTT, 20 μM cytochalasin B). The cells are the centrifuged at 1000 g for five minutes, the supernatant discarded, and the cells are resuspended in the original volume of KHM. The cells are then homogenized in a dounce homogenizer on ice with about 25 strokes and progress determined by microscopic observation. When at least 95% of the cells are homogenized extracts held on ice for use in envelope reassembly or cryopreserved as is well known in the art.

Preparation of Condensed Chromatin from Differentiated Cells

Donor dermal fibroblasts will be exposed to conditions that remove the plasma membrane, resulting in the isolation of nuclei. These nuclei, in turn, will be exposed to cell extracts that result in nuclear envelope dissolution and chromatin condensation. This results in the release of chromatin factors such as RNA, nuclear envelope proteins, and transcriptional regulators such as transcription factors that are deleterious to the reprogramming process. Dermal fibroblasts are cultured in DMEM with 10% fetal calf serum until the cells reach confluence. 1×10̂6 cells are then harvested by trypsinization as is well known in the art, the trypsin is inactivated, and the cells are suspended in 50 mL of phosphate buffered saline (PBS), pelleted by centrifuging the cells at 500 g for 10 minutes at 4° C., the PBS is discarded, and the cells are suspended in 50× the volume of the pellet in ice-cold PBS, and centrifuged as above. Following this centrifugation, the supernatant is discarded and the pellet is resuspended in 50× the volume of the pellet of hypotonic buffer (10 mM HEPES, pH 7.5, 2 mM MgCl₂, 25 mM KCl, 1 mM DTT, 10 μM aprotinin, 10 μM leupeptin, 10 μM pepstatin A, 10 μM soybean trypsin inhibitor, and 100 μM PMSF) and again centrifuged at 500 g for 10 min at 4 degrees C. The supernatant is discarded and 20× the volume of the pellet of hypotonic buffer is added and the cells are carefully resuspended and incubated on ice for an hour. The cells are then physically lysed using procedures well-known in the art. Briefly, 5 ml of the cell suspension is placed in a glass Dounce homogenizer and homogenized with 20 strokes. Cell lysis is monitored microscopically to observe the point where isolated and yet undamaged nuclei result. Sucrose is added to make a final concentration of 250 mM sucrose (1/8 volume of 2 M stock solution in hypotonic buffer). The solution is carefully mixed by gentle inversion and then centrifuged at 400 g at 4° C. for 30 minutes. The supernatant is discarded and the nuclei are then gently resuspended in 20 volumes of nuclear buffer (10 mM HEPES, pH 7.5, 2 mM MgCl₂, 250 mM sucrose, 25 mM KCl, 1 mM DTT, 10 μM aprotinin, 10 μM leupeptin, 10 μM pepstatin A, 10 μM soybean trypsin inhibitor, and 100 μM PMSF). The nuclei are re-centrifuged as above and resuspended in 2× the volume of the pellet in nuclear buffer. The resulting nuclei may then be used directly in nuclear remodeling as described below or cryopreserved for future use.

Preparation of Condensation Extract

The condensation extract, when added to the isolated differentiated cell nuclei, will result in nuclear envelope breakdown and the condensation of chromatin. Since the purpose of step 1 is to remodel the nuclear components of a somatic differentiated cell with that of an undifferentiated cell, the condensation extract used in this example is obtained from NTera-2 cells which are also the cells used to derive the extract for nuclear envelope reconstitution above. This results in a dilution of the components from the differentiated cell in extracts which contain the corresponding components desirable in reprogramming cells to an undifferentiated state. NTera-2 cl. Dl cells are easily obtained from sources such as the American Type Culture Collection (CRL-1973) and are grown at 37° C. in monolayer culture in DMEM with 4 mM L-glutamine, 1.5 g/L sodium bicarbonate and 4.5 g/L glucose, 10% fetal bovine serum (complete medium). While in a log growth state, the cells are plated at 5×10̂6 cells per sq cm tissue culture flask in 200 mL of complete medium. Methods of obtaining extracts capable of inducing nuclear envelope breakdown and chromosome condensation are well known in the art (Collas et al., J. Cell Biol. 147:1167-1180, (1999)). Briefly, NTera-2 cells in log growth as described above are synchronized in mitosis by incubation in 1 μg/ml nocodazole for 20 hours. The cells that are in the mitotic phase of the cell cycle are detached by mitotic shakeoff. The harvested detached cells are centrifuged at 500 g for 10 minutes at 4 degrees C. Cells are resuspended in 50 ml of cold PBS, and centrifuged at 500 g for an additional 10 min. at 4° C. This PBS washing step is repeated once more. The cell pellet is then resuspended in 20 volumes of ice-cold cell lysis buffer (20 mM HEPES, pH 8.2, 5 mM MgCl₂, 10 mM EDTA, 1 mM DTT, 10 μM aprotinin, 10 μM leupeptin, 10 μM pepstatin A, 10 μM soybean trypsin inhibitor, 100 μM PMSF, and 20 μg/ml cytochalasin B, and the cells are centrifuged at 800 g for 10 minutes at 4 degrees C. The supernatant is discarded, and the cell pellet is carefully resuspended in one volume of cell lysis buffer. The cells are placed on ice for one hour then lysed with a Dounce homogenizer. Progress is monitored by microscopic analysis until over 90% of cells and cell nuclei are lysed. The resulting lysate is centrifuged at 15,000 g for 15 minutes at 4 degrees C., the tubes are then removed and immediately placed on ice. The supernatant is gently removed using a small caliber pipette tip, and the supernatant from several tubes is pooled on ice. If not used immediately, the extracts are immediately flash-frozen on liquid nitrogen and stored at −80 degrees C. until use. The cell extract is then placed in an ultracentrifuge tube and centrifuged at 200,000 g for three hours at 4 degrees C. to sediment nuclear membrane vesicles. The supernatant is then gently removed and placed in a tube on ice and used immediately to prepare condensed chromatin or cryopreserved as described above.

Methods of Use of Condensation Extract

If beginning with a frozen aliquot on condensation extract, the frozen extract is thawed on ice. Then an ATP-generating system is added to the extract such that the final concentrations are 1 mM ATP, 10 mM creatine phosphate, and 25 μg/ml creatine kinase. The nuclei isolated from the differentiated cells as described above are then added to the extract at 2,000 nuclei per 10 μl of extract, mixed gently, the incubated in a 37° C. water bath. The tube is removed occasionally to gently resuspend the cells tapping on the tube. Extracts and cell sources vary in times for nuclear envelope breakdown and chromosome condensation. The progress is therefore monitored by periodic’ monitoring samples microscopically. When the majority of cells have lost their nuclear envelope and there is evidence of the beginning of chromosome condensation, the extract containing the condensing chromosome masses is placed in a centrifuge tube with an equal volume of 1 M sucrose solution in nuclear buffer. The chromatin masses are sedimented by centrifugation at 1,000 g for 20 minutes at 4° C. The supernatant is discarded, and the chromatin masses are gently resuspended in nuclear remodeling extract derived above. The sample is then incubated in a water bath at 33 degrees C. for up to two hours and periodically monitored microscopically for formation of remodeled nuclear envelopes around the condensed and remodeled chromatin as described (Burke & Gerace, Cell 44:639-652, (1986). Once a large percentage of chromatin has been encapsulated in nuclear envelopes, the remodeled nuclei may be used in cellular reconstitution using any of the techniques described in the present method.

Modification of Cell Extracts

As an optional modification to the methods disclosed herein, one or more factors are expressed or overexpressed in the undifferentiated cells (for example, in EC or other cells) used to obtain the nuclear remodeling and/or condensation extracts. Such factors include, for example, SOX2, NANOG, cMYC, OCT4, DNMT3B, embryonic histones, as well as other factors listed in Table 7 below and regulatory RNA that induce or increase the expression of proteins expressed in undifferentiated cells and that improve the frequency of reprogramming. Any combinations of the above-mentioned factors may be used. For example, undifferentiated cells of the present method may be modified to have increased expression of two, three, four, or more of any of the factors listed in Table 7. Alternatively, the level of one or more factors in the undifferentiated cells used to obtain the nuclear remodeling extract may be decreased relative to the levels found in unmodified cells. Such decreases in the level of a cell factor may be achieved by known methods, such as, for example, by use of transcriptional regulators, regulatory RNA, or antibodies specific for the cell factor.

Gene constructs encoding the proteins listed in Table 7 or their non-human homologues, or regulatory proteins or RNAs that alter expression of these factors, are transfected into the cells by standard techniques. Alternatively, recombinant proteins or other agents are directly added to the extracts.

### Example 16

### Genetic Modification of Remodeled Nuclei or Chromatin

The isolated nuclei or condensed chromatin may optionally be modified by methods involving recombinase treated targeting vectors or oligonucleotides. The DNA from cell free chromosomes and chromatin can be genetically modified enzymatically with targeting vectors or oligonucleotides, using purified recombinases, purified DNA repair proteins, or protein or cell extract preparations comprising such proteins. The targeting DNAs may have tens of kilobase pairs to oligonucleotides of at least 50 base pairs of homology to the chromosomal target. Recombinase catalyzed recombination intermediates formed between target chromosomes and vector DNA can be enzymatically resolved in cell free extracts with other purified recombination or DNA repair proteins to produce genetically modified chromosomes. These modified chromosomes can be reintroduced into cells or used in the formation of nuclei in vitro prior to introduction into cells/modified condensed chromatin can be used in nuclear envelope reconstitution (see step 2 below). Recombinase treated vector or oligonucleotides can also be directly introduced into isolated nuclei by microinjection or by diffusion into permeabilized nuclei to allow in situ formation of recombination intermediates that can be resolved in vitro, upon nuclear transfer into intact cells, or upon fusion with recipient cells.

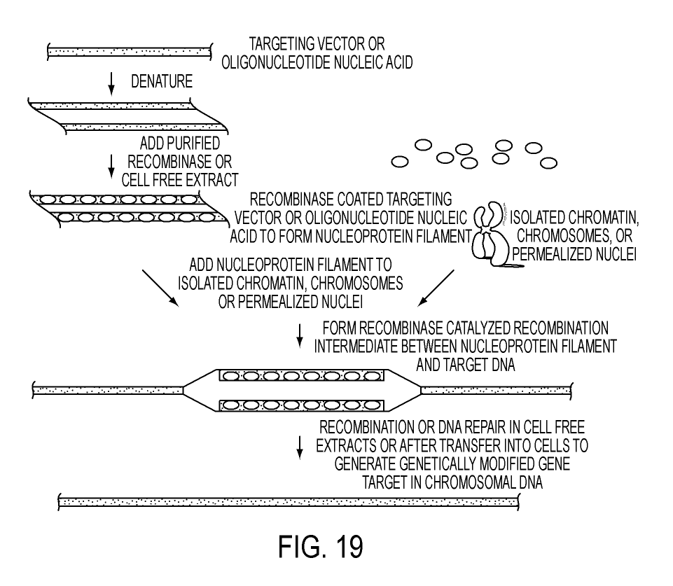

In this approach, enzymatically active nucleoprotein filaments are first formed between targeting vector, or oligonucleotides, and recombinase proteins. Recombinase proteins are cellular proteins that catalyze the formation of heteroduplex recombination intermediates intracellularly and can form similar intermediates in cell free systems. Well studied, prototype recombinases are the RecA protein from E. coli and Rad51 protein from eukaryotic organisms. Recombinase proteins cooperatively bind single stranded DNA and actively catalyze the search for homologous DNA sequences on other target chromosomal DNAs. Heteroduplex structures may also be formed and resolved using cell free extracts from cells with recombinogenic phenotypes (e.g., DT40 extracts). In a second step, heteroduplex intermediates may be resolved in cell free extracts by treatment with purified recombination and DNA repair proteins to recombine the donor targeting vector DNA or oligonucleotide into the target chromosomal DNA (FIG. 19). Resolution may also be accomplished using cell free extracts from normal cells or extracts from cells with a recombinogenic phenotype. Finally, the nuclear membrane is reformed around modified chromosomes and the remaining unmodified cellular chromosomal complement for introduction into recipient cells or oocytes.

Several techniques are available that can be used in gene modification of the reprogrammed cell. One technique is the Cre-Lox targeting system. Cre recombinase has been used to efficiently delete hundreds of base pairs to megabase pairs of DNA in mammalian cells. The LoxP and FRT recombinase recognition sequences allow recombinase mediated gene modifications of homologous recombinant cells.

Forming Recombinase Coated Nucleoprotein Filaments

Circular DNA targeting vectors are first linearized by treatment with restriction endonucleases, or alternatively linear DNA molecules are produced by PCR from genomic DNA or vector DNA. All DNA targeting vectors and traditional DNA constructs are removed from vector sequences by agarose gel electrophoresis and purified with Elutip-D columns (Schleicher & Schuell, Keene, N.H.). For RecA protein coating of DNA, linear, double-stranded DNA (200 ng) is heat denatured at 98 degrees C. for 5 minutes, cooled on ice for 1 minute and added to protein coating mix containing Tris-acetate buffer, 2 mM magnesium acetate and 2.4 mM ATP-gamma-S. RecA protein (8.4 μg) is immediately added, the reaction incubated at 37 degrees C. for 15 minutes, and magnesium acetate concentration increased to a final concentration of 11 mM. The RecA protein coating of DNA is monitored by agarose gel electrophoresis with uncoated double-stranded DNA as control. The electrophoretic mobility of RecA-DNA is significantly retarded as compared with non-coated double stranded DNA.

Isolation of cell free chromosomes and chromatin is achieved as described above. The condensation extract, when added to the isolated differentiated cell nuclei, will result in nuclear envelope breakdown and the condensation of chromatin. The resulting nuclei may then be used directly for gene modifications as just described, nuclear remodeling, or cryopreserved for future use. A separate extract is used for nuclear envelope reconstitution after cell free homologous recombination reactions have modified target chromosomes. Extract for nuclear envelope breakdown and chromatin condensation, and for nuclear envelope reconstitution may be prepared from any proficient mammalian cell line. However, extracts from the human embryonal carcinoma cell line NTera-2 can be potentially used for the condensation extract and for nuclear envelope reconstitution extract as well as for remodeling differentiated chromatin to an undifferentiated state, thus enhancing production of genetically modified human ES cells starting from differentiated human dermal cells.

Forming heteroduplex recombination intermediates between preformed recombinase coated nucleoprotein targeting vectors and oligonucleotides and cell free chromosomes and chromatin

Formation of targeting vector/chromosome heteroduplexes is performed by adding approximately 1-3 μg of double-stranded chromosomal DNA or chromatin masses to the RecA coated nucleoprotein filaments described above, and incubated at 37 degrees C. for 20 minutes. If the nucleoprotein heteroduplex structures are to be deproteinized prior to additional in vitro recombination steps, they are treated by with the addition of SDS to a final concentration of 1.2%, or by addition of proteinase K to 10 mg/ml with incubation for 15 to 20 minutes at 37 degrees C., followed by addition of SDS to a final concentration of 0.5 to 1.2% (wt/vol). Residual SDS is removed prior to subsequent steps by microdialysis against 100 to 1000 volumes of protein coating mix.

Resolving Recombination Intermediates with Cell Free Extracts

Cell free extracts may be prepared from normal fibroblast or hES cell lines, or may be prepared from cells demonstrated to have recombinogenic phenotypes. Cell lines exhibiting high levels of recombination in vivo are the chicken pre-B cell line DT40 and the human lymphoid DG75 cell line. Preparation of cell free extracts is performed at 4° C. About 10̂8 actively growing cells are harvested from either dishes or suspension cultures. The cells are washed three times with phosphate-buffered saline (PBS; 140 mM NaCl, 3 mM KCl, 8 mM NaH2PO4, 1 mM K2HPO4, 1 mM MgCl₂, 1 mM CaCl2), resuspended in 2 to 3 ml of hypotonic buffer A (10 mM Tris hydrochloride [pH 7.4], 10 mM MgCl₂, 10 mM KCl, 1 mM dithiothreitol), and kept on ice for 10 to 15 minutes. Phenylmethylsulfonyl fluoride is added to a concentration of 1 mM, and the cells are broken by 5 to 10 strokes in a Dounce homogenizer, pestle B. The released nuclei are centrifuged at 2,600 rpm in a Beckman TJ-6 centrifuge for 8 min. The supernatant is removed carefully and stored in 10% glycerol-100 mM NaCl at −70 degrees C. (cytoplasmic fraction). The nuclei are resuspended in 2 ml of buffer A containing 350 mM NaCl, and the following proteinase inhibitors are added: pepstatin to a concentration of 0.25 leupeptin to a concentration of 0.1 μg/ml, aprotinin to a concentration of 0.1 μg/ml, and phenylmethylsulfonyl fluoride to a concentration of 1 mM (all from Sigma Chemicals). After 1 h of incubation at 0 degrees C., the extracted nuclei are centrifuged at 70,000 rpm in a Beckman TL-100/3 rotor at 2 degrees C. The clear supernatant is adjusted to 10% glycerol, 10 mM β-mercaptoethanol and frozen immediately in liquid nitrogen prior to storage at −70 degrees C. (fraction 1).

To resolve recombination intermediates in vitro, chromosomal heteroduplex intermediates are incubated with 3 to 5 μg of extract protein in a reaction mixture containing 60 mM NaCl, 2 mM 2-mercaptoethanol, 2 mM KCl, 12 mM Tris hydrochloride (pH 7.4), 1 mM ATP, 0.1 mM each deoxyribonucleoside triphosphate (dNTP), 2.5 mM creatine phosphate, 12 mM MgCl₂, 0.1 mM spermidine, 2% glycerol, and 0.2 mM dithiothreitol. After 30 minutes at 37 degrees C., the reaction is stopped by the addition of EDTA to a concentration of 25 μM, sodium dodecyl sulfate (SDS) to a concentration of 0.5%, and 20 μg of proteinase K and incubated for 1 hour at 37 degrees C. SDS is removed prior to subsequent steps by microdialysis. An equal volume of 1 M sucrose is added to the treated chromatin masses and sedimented by centrifugation at 1,000×g for 20 minutes at 4 degrees C.

Reforming Nuclear Envelopes Around Recombinant Chromosomes and Chromatin

The supernatant is discarded, and the chromatin masses are gently resuspended in nuclear remodeling extract described above. The sample is then incubated in a water bath at 33 degrees C. for up to two hours and periodically monitored microscopically for the formation of remodeled nuclear envelopes around the condensed and remodeled chromatin as described (Burke & Gerace, Cell 44:639-652, (1986). Once a large percentage of chromatin has been encapsulated in nuclear envelopes, the remodeled nuclei may be used for cellular reconstitution using any of the techniques described in the present method.

Detection of Cells Containing Genetically Modified Chromosomes

Reconstituted cells are grown for 7 to 14 days and screened for recombinants using PCR and Southern hybridization.

### Example 17

### Modification of Chromosomes and Chromatin in Isolated Nuclei with Targeting Vectors or Oligonucleotides to Engineer Cells

Chromosomes and chromatin may be genetically modified in isolated nuclei from cells. In this approach, intact nuclei are isolated from growing cells, and reversibly permeabilized to allow diffusion of nucleoprotein targeting vectors and oligonucleotides into the nucleus interior. Heteroduplex intermediates formed between nucleoprotein targeting vectors and oligonucleotides and chromosomal DNA may be resolved by treatment with recombination proficient cell extracts, purified recombination and DNA repair proteins, or by cellular reconstitution with the nuclei into recombination proficient cells.

Isolation and Permeabilization of Nuclei

Preparation of Synchronous Populations of Nuclei Cell Culture and Synchronization are carried out as previously described (Leno et al., Cell 69:151-158 (1992)). Nuclei are prepared as described except that all incubations are carried out in HE buffer (50 mM Hepes-KOH, pH 7.4, 50 mM KCl, 5 mM MgCl₂, 1 mM EGTA, 1 mM DTT, 1 μg/ml aprotinin, pepstatin, leupeptin, chymostatin).

Nuclear Membrane Permeabilization Streptolysin O (SLO)-prepared nuclei (Leno et al., Cell 69:151-158 (1992)) are incubated with 20 μg/ml lysolecithin (Sigma Immunochemicals) and 10 μg/ml cytochalasin B in HE at a concentration of ˜1.5×10̂4 nuclei/ml for 10 min at 23 degrees C. with occasional gentle mixing. Reactions are stopped by the addition of 1% nuclease free BSA (Sigma Immunochemicals). Nuclei are gently pelleted by centrifugation in a RC5B rotor (Sorvall Instruments, Newton, Conn.) at 500 rpm for 5 min and then washed three times by dilution in 1 ml HE. Pelleted nuclei are recovered in a small volume of buffer and resuspended to ˜1×10̂4 nuclei/μl.

Forming heteroduplex recombination intermediates between preformed recombinase coated nucleoprotein targeting vectors and oligonucleotides and cell free chromosomes and chromatin

Formation of targeting vector/chromosome heteroduplexes is performed by adding approximately 1×10̂5 to 1×10̂6 permeabilized nuclei to the RecA coated nucleoprotein filaments described above, and incubated at 37 degrees C. for 20 minutes.

Resolving Recombination Intermediates with Cell Free Extracts

Cell free extracts may be prepared from normal fibroblast or hES cell lines, or may be prepared from cells demonstrated to have recombinogenic phenotypes. Cell lines exhibiting high levels of recombination in vivo are the chicken pre-B cell line DT40 and the human lymphoid DG75 cell line. Preparation of cell free extracts are performed at 4 degrees C. About 10̂8

actively growing cells are harvested from either dishes or suspension cultures. The cells are washed three times with phosphate-buffered saline (PBS; 140 mM NaCl, 3 mM KCl, 8 mM NaH2PO4, 1 mM K2HPO4, 1 mM MgCl₂, 1 mM CaCl2), resuspended in 2 to 3 ml of hypotonic buffer A (10 mM Tris hydrochloride [pH 7.4], 10 mM MgCl₂, 10 mM KCl, 1 mM dithiothreitol), and kept on ice for 10 to 15 minutes. Phenylmethylsulfonyl fluoride is added to 1 mM, and the cells are broken by 5 to 10 strokes in a Dounce homogenizer, pestle B. The released nuclei are centrifuged at 2,600 rpm in a Beckman TJ-6 centrifuge for 8 min. The supernatant is removed carefully and stored in 10% glycerol-100 mM NaCl at −70 degrees C. (cytoplasmic fraction). The nuclei are resuspended in 2 ml of buffer A containing 350 mM NaCl, and the following proteinase inhibitors are added: pepstatin to 0.25 μg/ml, leupeptin to 0.1 μg/ml, aprotinin to 0.1 μg/ml, and phenylmethylsulfonyl fluoride to 1 mM (all from Sigma Chemicals). After 1 h of incubation at 0 degrees C., the extracted nuclei are centrifuged at 70,000 rpm in a Beckman TL-100/3 rotor at 2 degrees C. The clear supernatant is adjusted to 10% glycerol, 10 mM β-mercaptoethanol and frozen immediately in liquid nitrogen prior to storage at −70 degrees C. (fraction 1).

To resolve recombination intermediates in permeabilized nuclei, nuclei containing chromosomal heteroduplex intermediates are incubated with 3 to 5 μg of extract protein in a reaction mixture containing 60 mM NaCl, 2 mM 3-mercaptoethanol, 2 mM KCl, 12 mM Tris hydrochloride (pH 7.4), 1 mM ATP, 0.1 mM each deoxyribonucleoside triphosphate (dNTP), 2.5 mM creatine phosphate, 12 mM MgCl₂, 0.1 mM spermidine, 2% glycerol, and 0.2 mM dithiothreitol. After 30 minutes at 37 degrees C., the reaction is stopped.

Nuclear Envelope Repair

Preparation and Fractionation of Nuclear Repair Extract

Low-speed Xenopus egg extracts (LSS) 1 are prepared essentially according to the procedure described by Blow and Laskey Cell 21; 47:577-87 (1986)). Extraction buffer (50 mM Hepes-KOH, pH 7.4, 50 mM KCl, 5 mM MgCl2) is thawed and supplemented with 1 mM DTT, 1 μg/ml leupeptin, pepstatin A, chymostatin, aprotinin, and 10 μg/ml cytochalasin B (Sigma Immunochemicals, St. Louis, Mo.) immediately before use. Extracts are supplemented with 2% glycerol and snap-frozen as 10-20 μl beads in liquid nitrogen or subjected to further fractionation. High speed supernatant (HSS) and membrane fractious are prepared from low-speed egg extract as described (Sheehan et al., J Cell Biol. 106:1-12 (1988)). Membranous material, isolated by centrifugation of 1-2 ml of low-speed extract, is washed at least two times by dilution in 5 ml extraction buffer. Diluted membranes are centrifuged for 10 minutes at 10 k rpm in an SW50 rotor (SW50; Beckman Instruments, Inc., Palo Alto, Calif.) to yield vesicle fraction 1. The supernatant is then centrifuged for a further 30 min at 30 k rpm to yield vesicle fraction 2. Washed membranes are supplemented with 5% glycerol and snap-frozen in 5 μl beads in liquid nitrogen. Vesicle fractions 1 and 2 are mixed in equal proportions before use in nuclear membrane repair reactions.

Treatment for Nuclear Envelope Repair

Lysolecithin-permeabilized nuclei are repaired by incubation with membrane components prepared from Xenopus egg extracts. Nuclei at a concentration of approximately 5000/μl are mixed with an equal volume of pooled vesicular fractions 1 and 2 and supplemented with 1 mM GTP and ATP. 10-20 μl reactions are incubated at 23 degrees C. for up to 90 min with occasional gentle mixing. Aliquots are taken at intervals and assayed for nuclear permeability.

Once a large percentage of chromatin is encapsulated in nuclear envelopes, the remodeled nuclei may be used for cellular reconstitution using any of the techniques described in the present method.

Detection of Cells Containing Genetically Modified Chromosomes

Reconstituted cells are grown for 7 to 14 days and screened for recombinants using PCR and Southern hybridization.

### Example 18

### Modification of Isolated Chromosomes, Chromatin, and Nuclei Using Cell Free Extracts to Engineer Cells with Exogenous Genetic Material

In this approach, targeting vectors or oligonucleotides and the target chromosomal DNA are directly treated with recombination proficient cell free extracts from cells with recombinogenic phenotypes such as the chicken pre-B cell line DT40 and the human lymphoid cell line DG75. These cell free extracts may be used on isolated chromosome and chromatin or on isolated permeabilized nuclei. Essentially, targeting vector/oligonucleotides are incubated with isolated chromosomes, chromatin, or nuclei and cell free recombination extract. The nuclear envelope is reconstituted around recombinant chromosomes or chromatin, or the nuclear envelope of recombinant, permeabilized, nuclei are repaired prior to cell reconstitution with the reconstituted or repaired nuclei.

Preparation of Cell Free Extracts

Cell free extracts from DT40 or DG75 cells are prepared as described above.

Preparation of Chromosomes, Chromatin, or Nuclei

Isolated chromosomes, chromatin, and permeabilized nuclei from fibroblasts, hES cell lines, or germ cell lines are as described above.

Recombination between targeting vectors and oligonucleotides, and cell free chromosomes and chromatin using cell free extracts from recombinogenic cells

Circular DNA targeting vectors are first linearized by treatment with restriction endonucleases, or alternatively linear DMA molecules are produced by PCR from genomic DNA or vector DNA. All DNA targeting vectors and traditional DNA constructs are removed from vector sequences by agarose gel electrophoresis and purified with Elutip-D columns (Schleicher & Schuell, Keene, N.H.). Double-stranded DNA (200 ng) is heat denatured at 98 degrees C. for 5 minutes, cooled on ice for 1 minute and added to approximately 1-3 μg of double-stranded chromosomal DNA or chromatin masses, or approximately 1×10̂5 to 1×10̂6 permeabilized nuclei, and 3 to 5 μg of extract protein in a reaction mixture containing 60 mM NaCl, 2 mM 2-mercaptoethanol, 2 mM KCl, 12 mM Tris hydrochloride (pH 7.4), 1 mM ATP, 0.1 mM each deoxyribonucleoside triphosphate (dNTP), 2.5 mM creatine phosphate, 12 mM MgCl₂, 0.1 mM spermidine, 2% glycerol, and 0.2 mM dithiothreitol. The reaction mixtures are incubated at 37° C. for at least 30 minutes are processed as describe above prior to reconstituting cellular envelopes or repairing permeabilized nuclei.

Reforming Nuclear Envelopes Around Recombinant Chromosomes and Chromatin

Nuclear envelopes are reconstituted around recombinant chromosomes and chromatin and reconstituted nuclei used for cellular reconstitution as describe above.

Nuclear Envelope Repair

Recombinant, permeabilized nuclei are repaired and repaired recombinant nuclei used for cellular reconstitution as described above.

Detection of Cells Containing Genetically Modified Chromosomes

Reconstituted cells are grown for 7 to 14 days and screened for recombinants using PCR and Southern hybridization.

### Example 19

### Modification of Chromosomes and Chromatin in Intact Cells with Recombinase Treated Targeting Vectors or Oligonucleotides to Engineer Cells with Exogenous Genetic Material

In this approach, double stranded targeting vectors, targeting DNA fragments, or oligonucleotides are coated with bacterial or eukaryotic recombinase and introduced into mammalian cells or oocytes. The activated nucleoprotein filament forms heteroduplex recombination intermediates with the chromosomal target DNA that is subsequently resolved to a homologous recombinant structure by the cellular homologous recombination or DNA repair pathways. While the most direct delivery of nucleoprotein filaments is by direct nuclear/pronuclear microinjection, other delivery technologies can be used including electroporation, chemical transfection, and single cell electroporation.

To form human Rad51 nucleoprotein filaments, linear, double-stranded DNA (200 ng) is heat denatured at 98 degrees C. for 5 minutes, cooled on ice for 1 minute and added to a protein coating mix containing 25 mM Tris acetate (pH 7.5), 100 μg/ml BSA, 1 mM DTT, 20 mM KCl (added with the protein stock), 1 mM ATP and 5 mM CaCl2, or AMP-PNP and 5 mM MgCl2. hRad51 protein (1 μM) is immediately added and the reaction incubated for 10 minutes at 37 degrees C. The hRad51 protein coating of the DNA is monitored by agarose gel electrophoresis with uncoated double-stranded DNA as control. The electrophoretic mobility of hRad51-DNA nucleoprotein filament is significantly retarded as compared with non-coated double stranded DNA. hRad51-DNA nucleoprotein filaments are diluted to a concentration of 5 ng/μl and used for nuclear microinjection of human fibroblasts or somatic cells, or used for pronuclear microinjection of activated oocytes created by somatic cell nuclear transfer or in vitro fertilization.

Detection of Cells Containing Genetically Modified Chromosomes

Injected cells or oocytes are grown for 7 to 14 days and screened for recombinants using PCR and Southern hybridization.

### Example 20

### Cellular Reconstitution

Step 2, also referred to as “cellular reconstitution” in the present method is carried out using nuclei or chromatin remodeled by any of the techniques described in the present disclosure, such as in Examples 14 and 15 above or combinations of the techniques described in Examples 14 and 15 as described more fully in the present disclosure. During cellular reconstitution in this example, the remodeled nuclei are fused with enucleated cytoplasts of hES cells as is known in the art (Do & Scholer, Stem Cells 22:941-949 (2004)). Briefly, the human ES Cell line H9 is cultured under standard conditions (Klimanskaya et al., Lancet 365: 4997 (1995)). The cytoplasmic volume of the cells is increased by adding 10 μM cytochalasin B for 20 hours prior to manipulation. Cytoplasts are prepared by centrifuging trypsinized cells through a Ficoll density gradient using a stock solution of autoclaved 50% (wt/vol) Ficoll-400 solution in water. The stock Ficoll 400 solution is diluted in DMEM and with a final concentration of 10 μM cytochalasin B. The cells are centrifuged through a gradient of 30%, 25%, 22%, 18%, and 15% Ficoll-400 solution at 36° C. Layered on top is 0.5 mL of 12.5% Ficoll-400 solution with 10×10̂6 ES cells. The cells are centrifuged at 40,000 rpm at 36 degrees C. in an MLS-50 rotor for 30 minutes. The cytoplasts are collected from the 15% and 18% gradient regions marked on the tubes, rinsed in PBS, and mixed on a 1:1 ratio with remodeled nuclei from step one of the present method or cryopreserved. Fusion of the cytoplasts with the nuclei is performed using polyethylene glycol (see Pontecorvo “Polyethylene Glycol (PEG) in the Production of Mammalian Somatic Cell Hybrids” Cytogenet Cell Genet. 16 (1-5):399-400 (1976), briefly in 1 mL of prewarmed 50% polyethylene glycol 1500 (Roche) for one minute. 20 mL of DMEM was then added over a five minute period to slowly remove the polyethylene glycol. The cells were centrifuged once at 130 g for five minutes and then taken back up in 50 μL of ES cell culture medium and placed beneath a feeder layer of fibroblasts under conditions to promote the outgrowth of an ES cell colony.

### Example 21

### Cellular Reconstitution

Step 2, also referred to as “cellular reconstitution” in the present method is also carried out using nuclei remodeled by any of the techniques described in the present disclosure, in this example as in Example 15 above and the cellular reconstitution step that follows. The nuclei are fused with anucleate cytoplasmic blebs of hES cells as is well known in the art (Wright & Hayflick, Exp. Cell Res. 96:113-121, (1975); & Wright & Hayflick, Proc. Natl. Acad. Sci., USA, 72:1812-1816, (1975). Briefly, the cytoplasmic volume of the hES cells is increased by adding 10 μM cytochalasin B for 20 hours prior to manipulation. The cells are trypsinized and replated on sterile 18 mm coverslips coated with mouse embryonic fibroblast feeder extracellular matrix as described (Klimanskaya et al., Lancet 365: 4997 (2005). The cells are plated at a density such that after an overnight incubation at 37° C. and one gentle wash with medium, the cells cover about 90% of the surface area of the coverslip. The coverslips are then placed face down in a centrifuge tube containing 8 mL of 10% Ficoll-400 solution and centrifuged at 20,000 g at 36° C. for 60 minutes. Remodeled nuclei resulting from step one of the present method are then spread onto the coverslip with a density of at least that of the cytoplasts, preferable at least five times the density of the cytoplasts. Fusion of the cytoplasts with the nuclei is performed using polyethylene glycol (see Pontecorvo “Polyethylene Glycol (PEG) in the Production of Mammalian Somatic Cell Hybrids” Cytogenet Cell Genet. 16 (1-5)-399-400 (1976). Briefly, in 1 mL of prewarmed 50% polyethylene glycol 1500 (Roche) in culture medium is placed over the coverslip for one minute. 20 mL of culture medium is then added drip-wise over a five minute period to slowly remove the polyethylene glycol. The entire media is then aspirated and replaced with culture medium.

### Example 22

### Analysis of the Molecular Mechanisms of Reprogramming

The in vitro remodeling of somatic cell-derived DNA as described in example 15 of the present method is utilized as a model of the reprogramming of a somatic cell and an assay useful in analyzing the molecular mechanisms of reprogramming. The protocol of example 15 is followed to the time immediately preceding that when extracts from mitotic NTera2 cells are added. Prior to the addition of mitotic NTera2 cell extract, purified lamin A protein from human skin fibroblasts is added in amounts corresponding to 10̂-6, 10̂-4, 10̂-3, 10̂-2, 10̂-1, 1× and 10× the concentration in human fibroblast mitotic cell extract. The lamin A reduces the extent of successful reprogramming following step 2 cellular reconstitution, and the use of this assay system determines the extent of lamin A interference in successful reprogramming.

### Example 23

### Reprogramming Factors

The frequency of obtaining reprogrammed cells may be improved by increasing the expression of undifferentiated cell factors in the undifferentiated cells or cell extracts of steps 1 and 2 of the present method. These factors may be introduced into the extracts of step 1, or into the enucleated cytoplasts of step 2 using techniques well known in the art and described herein. The final concentration of said factor should be at least the concentration observed in cultures of human ES cells grown under standard conditions, or preferably 2-50-fold higher in concentration than that observed in said standard hES cell cultures. Table 7 provides a list of exemplary undifferentiated cell factors. The table provides the names and accession names for the human genes; however homologues found in other species may also be used:

TABLE 7
List of exemplary undifferentiated cell factors

| | |
|---|---|
| BARX1 | NM_021570.2 |
| CROC4 | NM_006365.1 |
| DNMT3B | NM_175849.1 |
| H2AFX | NM_002105.1 |
| HHEX | NM_002729.2 |
| HIST1H2AB | NM_003513.2 |
| HIST1H4J | NM_021968.3 |
| HMGB2 | NM_002129.2 |
| hsa-miR-18a | MI0000072 |
| hsa-miR-18b | MI0001518 |
| hsa-miR-20b | MI0001519 |
| hsa-miR-106a | MI0000113 |
| hsa-miR-107 | MI0000114 |
| hsa-miR-141 | MI0000457 |
| hsa-miR-183 | MI0000273 |
| hsa-miR-187 | MI0000274 |
| hsa-miR-203 | MI0000283 |
| hsa-miR-211 | MI0000287 |
| hsa-miR-217 | MI0000293 |
| hsa-miR-218-1 | MI0000294 |
| hsa-miR-218-2 | MI0000295 |
| hsa-miR-302a | MI0000738 |
| hsa-miR-302c | MI0000773 |
| hsa-miR-302d | MI0000774 |
| hsa-miR-330 | MI0000803 |
| hsa-miR-363 | MI0000764 |
| hsa-miR-367 | MI0000775 |
| hsa-miR-371 | MI0000779 |
| hsa-miR-372 | MI0000780 |
| hsa-miR-373 | MI0000781 |
| hsa-miR-496 | MI0003136 |
| hsa-miR-508 | MI0003195 |
| hsa-miR-512-3p | |
| hsa-miR-512-5p | |
| hsa-miR-515-3p | |
| hsa-miR-515-5p | |
| hsa-miR-516-5p | |
| hsa-miR-517 | |
| hsa-miR-517a | MI0003161 |
| hsa-miR-518b | MI0003156 |
| hsa-miR-518c | MI0003159 |
| hsa-miR-518e | MI0003169 |
| hsa-miR-519e | MI0003145 |
| hsa-miR-520a | MI0003149 |
| hsa-miR-520b | MI0003155 |
| hsa-miR-520e | MI0003143 |
| hsa-miR-520g | MI0003166 |
| hsa-miR-520h | MI0003175 |
| hsa-miR-523 | MI0003153 |
| hsa-miR-524 | MI0003160 |
| hsa-miR-525 | MI0003152 |
| hsa-miR-526a-1 | MI0003157 |
| hsa-miR-526a-2 | MI0003168 |
| LEFTB | NM_020997.2 |
| LHX1 | NM_005568.2 |
| LHX6 | NM_014368.2 |
| LIN28 | NM_024674.3 |
| MYBL2 | NM_002466.2 |
| MYC | NM_002467.2 |
| MYCN | NM_005378.3 |
| NANOG | NM_024865.1 |
| NFIX | NM_002501.1 |
| OCT¾ (POU5F1) | NM_002701.2 |
| OCT6 (POU3F1) | NM_002699.2 |
| OTX2 | NM_172337.1 |
| PHC1 | NM_004426.1 |
| SALL4 | NM_020436.2 |
| SOX2 | NM_003106.2 |
| TERF1 | NM_003218.2 |
| TERT | NM_198254.1 |
| TGIF | NM_003244.2 |
| VENTX2 | NM_014468.2 |
| ZIC2 | NM_007129.2 |
| ZIC3 | NM_003413.2 |
| ZIC5 | NM_033132.2 |
| ZNF206 | NM_032805.1 |

Methods for expressing proteins or regulatory RNA that increases expression of these proteins within cells or means of introducing these factors into cellular extracts are well-known in the art and include a variety of techniques including without limitation:

Viral infection for stable and transient expression of proteins and regulatory RNAs, such viruses including without limitation: lentivirus bovine papilloma and other papilloma viruses, adenoviruses and adeno-associated viruses. In addition, the genes or RNAs may be introduced by transfection for transient and stable expression of proteins and regulatory RNAs through the use of plasmid vectors, mammalian artificial chromosomes BACS/PACS the direct addition of the proteins encoded in the listed genes, the miRNA or mRNA listed, using CaPO4 precipitate-mediated endocytosis, dendrimers, lipids, electroporation, microinjection, homologous recombination to modify the gene or its promoters or enhancers, chromosome-mediated gene transfer, cell fusion, microcell fusion, or the addition of cell extracts containing said useful factors, all of such techniques are well-known in the art and protocols for carrying out said techniques to administer said factors are readily available to researchers in the literature and internet.

### Example 24

### Induction Beta Cell Differentiation from Reprogrammed Cells without the Generation of ES Cell Lines

Peripheral blood nucleated cells are obtained from a patient in need of pancreatic beta cells. The cells are purified using flow cytometry to obtain monocytes using techniques well-known in the art.

Nuclei from the monocytes are then prepared by placing the cells in hypotonic buffer and dounce homogenizing the cells as is described in the art. The isolated monocyte nuclei from the patient are then exposed to a mitotic extract from the human EC cell line Tera-2 and incubated while monitoring samples of the extract to observed nuclear envelope breakdown and subsequent reformation of the nuclear envelope as described herein. The resulting reprogrammed cell nuclei are then fused with EC cell cytoplasts from the EC cell line Tera-2 that have been transfected with plasmids to overexpress the genes OCT4, SOX2, and NANOG as described herein. The resulting reconstituted cells in a heterogeneous mixture of reprogrammed and non-reprogrammed cells are then permeabilized and exposed to extracts of beta cells isolated from bovine pancreas as described herein and then directly differentiated into endodermal lineages without the production of an ES cell line. One million of the heterogeneous mixture of cells are then added onto mitotically-inactivated feeder cells that express high levels of NODAL or cell lines that express members of the TGF beta family that activate the same receptor as NODAL such as CM02 cells that express relatively high levels of Activin-A, but low levels of Inhibins or follistatin. The cells are then incubated for a period of five days in DMEM medium with 0.5% human serum. After five days, the resulting cells which include definitive endodermal cells are purified by flow cytometry or other affinity-based cell separation techniques such as magnetic bead sorting using antibody specific to the CXCR4 receptor and then cloned using techniques described in the pending patent applications PCT/US2006/013573 filed Apr. 11, 2006; and U.S. Application No. 60/811,908, filed Jun. 7, 2006, which are incorporated by means of reference. These cells are then directly differentiated into pancreatic beta cells or beta cell precursors using techniques known in the art for differentiating said cells from human embryonic stem cell lines or by culturing the cells on inducer cell mesodermal cell lines as described in PCT/US2006/013573 filed Apr. 11, 2006, and U.S. Application No. 60/811,908, filed Jun. 7, 2006, which are both incorporated by means of reference.

It is envisioned that the disclosed improved methods for the reprogramming of animal somatic cells are generally useful in mammalian and human cell therapy, such as human cells useful in treating dermatological, cardiovascular, neurological, endocrinological, skeletal, and blood cell disorders.

Each document cited herein is hereby incorporated by reference in its entirety to the extent that they are not inconsistent with the disclosures contained herein.

While the invention has been described by way of examples and preferred embodiments, it is understood that the words which have been used herein are words of description, rather than words of limitation. From the foregoing description, it will be apparent that variations and modifications may be made to the method described herein to adopt it to various usages and conditions. Changes may be made, within the purview of the appended claims, without departing from the scope and spirit of the invention in its broader aspects. Although the invention has been described herein with reference to particular means, materials, and embodiments, it is understood that the invention is not limited to the particulars disclosed. The invention extends to all equivalent structures, means, and uses which are within the scope of the appended claims.

1. A method of converting a non-multipotent or non-pluripotent recipient human or non-human animal somatic cell or nucleus thereof into a multipotent or pluripotent cell or into a nucleus or into a cell or nucleus of a different cell fate or lineage, comprising:
providing a non-multipotent or non-pluripotent recipient human or non-human animal somatic cell or a nucleus of a non-multipotent or non-pluripotent recipient human or non-human animal somatic cell; and
treating the recipient cell or the nucleus of said non-multipotent or non-pluripotent recipient human or non-human animal somatic cell with at least one reprogramming composition comprising at least one reprogramming factor for a time sufficient to convert the human or non-human animal recipient cell or a cytoplast containing the treated nucleus into a multipotent cell or into a cell of a different cell fate or lineage.

2. The method of claim 1, wherein at least one of said reprogramming factors is provided by a source cell that secretes at least one reprogramming factor into an aqueous medium containing said recipient cell or nucleus or wherein at least one of said reprogramming factors is provided by a cell extract obtained from a pluripotent cell.

3. (canceled)

4. (canceled)

5. (canceled)

6. The method of claim 1 wherein the reprogramming composition comprises an Oct4 polypeptide.

7. (canceled)

8. (canceled)

9. (canceled)

10. The method of claim 1, wherein said reprogramming factor is essentially free from viruses capable of genetically modifying said recipient cell.

11. The method of claim 1, wherein said reprogramming composition comprises a reprogramming polypeptide.

12. The method of claim 11, wherein said reprogramming polypeptide is essentially free from a polynucleotide that encodes said reprogramming polypeptide.

13. The method of claim 11, wherein said reprogramming polypeptide comprises at least one protein transduction domain that facilitates entry of the reprogramming polypeptide into the recipient cell and/or facilitates entry of the reprogramming polypeptide into the recipient cell nucleus.

14. The method of claim 13, wherein each protein transduction domain comprises a polypeptide independently selected from the group consisting of any of the polypeptides of SEQ ID NO: 1 through 10, and any combination thereof.

15. (canceled)

16. (canceled)

17. The method of claim 11, wherein the reprogramming composition comprises Oct4 and at least one reprogramming polypeptides selected from the group consisting of Nanog, c-Myc, Klf4, Sox2, and Lin28.

18. The method of claim 11, wherein said reprogramming polypeptide is comprised in a donor cell cytoplasm.

19. The method of claim 18 wherein said donor cell is selected from an oocyte, an inner cell mass cell, a morula cell, a blastocyst cell, an ES cell, an adult stem cell, and a primordial germ cell.

20. The method of claim 19, wherein said donor cell has been genetically modified to increase the expression of one or more reprogramming polypeptides selected from the group consisting of Nanog, c-Myc, Klf4, Sox2, and Lin28.

21. (canceled)

22. (canceled)

23. (canceled)

24. (canceled)

25. (canceled)

26. The method of claim 1, wherein said step of treating the recipient cell or nucleus with a reprogramming composition is selected from the group consisting of fusion with a liposome, fusion with an enucleated donor cell, fusion or contacting with a cytoplasmic bleb containing at least one reprogramming factor, electroporation, microinjection, and culturing the recipient cell or nucleus in a medium containing said reprogramming composition and optionally containing a cell and/or nucleus entry agent.

27. The method of claim 26, wherein the cell and/or nucleus entry agent is selected from the group consisting of Streptolysin O, digitonin, and a cationic amphiphile.

28. (canceled)

29. The method of claim 1, wherein the recipient cell is selected from the group consisting of a fibroblast, a neural cell, an astrocyte, a glial cell, and a Sox2 expressing cell.

30. (canceled)

31. (canceled)

32. A method of treating a disease, comprising:
providing a recipient cell or nucleus derived from a cell donor;
making the recipient cell or nucleus into a multipotent or pluripotent cell by the method of claim 1;
optionally, genetically modifying said multipotent or pluripotent cell;
optionally, treating said multipotent or pluripotent cell with a treatment that causes, facilitates, and/or potentiates differentiation into one or more desired cell types; and
introducing said multipotent or pluripotent cell or differentiated cell derived therefrom into a human or non-human animal patient in need thereof
wherein the multipotent or pluripotent cell is histocompatible with the patient.

33. (canceled)

34. (canceled)

35. (canceled)

36. A reprogramming composition, comprising at least two reprogramming polypeptides selected from the group consisting of Nanog, c-Myc, Oct4, Klf4, Sox2, and Lin28.

37. (canceled)

38. (canceled)

39. (canceled)

40. (canceled)

41. The reprogramming composition of claim 36, wherein said reprogramming polypeptides are comprised in a donor cell cytoplasm.

42. The reprogramming composition of claim 41, wherein said donor cell cytoplasm is derived from a cell selected from the group consisting of an unfertilized oocyte, a fertilized oocyte, an embryonic stem cell, an iPS cell, a teratoma cell, a blastomere, and an inner cell mass cell.

43. The reprogramming composition of claim 41, wherein said donor cell cytoplasm is derived from a cell that has been treated to cause expression of one or more reprogramming polypeptides selected from the group consisting of Nanog, c-Myc, Oct4, Klf4, Sox2, and Lin28.

44-156. (canceled)
